(19) United States
(12) Patent Application Publication
(10) Pub. No.: US 2005/0143349 A1
(43) Pub. Date: Jun. 30, 2005

(54) Phosphorus containing compounds useful for the regulation of LDL and Lp(a) levels

(75) Inventor: Alexander Bridges, Saline, MI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: QUATRx Pharmaceuticals Co.

(21) Appl. No.: 11/011,965

(22) Filed: Dec. 14, 2004

Related U.S. Application Data
(60) Provisional application No. 60/530,225, filed on Dec. 17, 2003.

(51) Int. Cl. A61K 31/675

(52) U.S. Cl. 514/89; 514/92; 514/93; 546/22; 514/85; 514/86; 544/243

(57) ABSTRACT

The invention provides phosphorus containing compounds, pharmaceutical compositions comprising the compounds, and methods of administering the compounds to a subject, wherein the phosphorus containing compounds are of the Formula I: 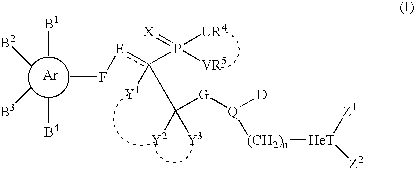 wherein
B¹, B², B³, B⁴, Ar, F, E, G, Y¹, Y², Y³, X, U, V, R⁴, R⁵, Q, D, n, Het, Z¹, and Z², are as defined herein.

### CROSS REFERENCE TO RELATED APPLICATIONS

This application claims the benefit of U.S. provisional application 60/530,225, filed Dec. 17, 2003.

### FIELD OF THE INVENTION

The invention relates to phosphorus containing compounds, pharmaceutical compositions comprising the compounds, and uses thereof. More specifically, the invention relates to aryl and heteroaryl phosphonates, processes for their preparation, pharmaceutical compositions comprising them, and their use in methods of regulating plasma levels of LDL and Lp(a).

### BACKGROUND OF THE INVENTION

Compounds that can bind as ligands for a variety of nuclear hormone receptors, particularly to a sub-family of nuclear hormone receptors that are activated by ligand binding and heterodimerization with retinoid X receptor (RXR) nuclear hormone receptors can be effective in regulating metabolic processes. Such receptors include peroxisome proliferator activated receptor (PPARα, PPARβ, PPARγ), famesoid X receptor (FXR), liver X receptor (LXRα, LXRβ), the pregnane X receptor (PXR), vitamin D receptor (VDR), thyroid hormone receptor (TRα, TRβ), and the retinoic acid receptors (RARs). These receptors are important regulators of metabolism, and are involved in multiple signal transduction pathways. Therefore compounds that modulate these receptors can be useful drugs for treatment of a wide range of metabolic disorders, including, for example, hypercholesteremia, hypertriglyceridemia, low HDL-C atherosclerosis, hyperglycemia, syndrome X, hyperinsulinemia, and diabetes.

Phosphonate compounds have been disclosed as useful for regulating the amount of Lipoprotein (a) [Lp(a)], an LDL-like lipoprotein wherein the major lipoprotein, apo B-100, is covalently linked to an unusual glycoprotein, apolipoprotein(a), in blood. [Phan, et al., WO 02/26752]. The covalent association between apo(a) and apo B to form Lp(a) is a secondary event which is independent of the plasma concentration of apo B. Apo(a) is an unusual protein, consisting mainly of a highly variable number (10-50) of so called “kringle” domains. Due to the structural similarity to the kringle domains in plasminogen and the plasminogen activators, apo(a) interferes with the normal physiological thrombosis-hemostasis process by preventing thrombolysis, that is clot dissolution. [Biemond et al. Circulation, 1997, 96(5): 1612-1615].

Elevated levels of Lp(a) have been associated with the development of atherosclerosis, coronary heart disease, myocardial infarction, cerebral infarction, and stroke. [Framingham Heart Study, Boscom et al. J. Amer. Med. Assoc. 1996, 276 (7): 544-548. Scanu, A. M. J. intern. Med. 231(6): 679-683. Stein et al. Arch. Intern. Med., 1997, 157 (11): 1170-1176.]Some, but not all, epidemiologic studies have strongly suggested a positive correlation between plasma Lp(a) concentrations and the incidence of restenosis following balloon angioplasty. [Desmarais et al. Circulation, 1995, 91 (5): 1403-1409. Igarishi et al. Circulation J., 2003 67 (7): 605-611. Miyata et al. Am. Heart J., 1996, 132 (2 Pt 1): 269-273. Hearn et al. Am, J. Cardiol., 1992, 69 (8): 736-739.]

Patients that have Lp(a) levels in excess of 20-30 mg/dL run a significantly increased risk of heart attacks and stroke. An effective therapy for lowering Lp(a) does not exist at present because cholesterol lowering agents such as the HMGCoA reductase inhibitors do not lower Lp(a) plasma concentrations. The only compound that lowers Lp(a) is niacin, but the high doses necessary for activity are accompanied with unacceptable side-effects. Thus, there is also a need for additional agents that effectively reduce elevated levels of Lp(a), as well as for agents that can bind to nuclear hormone receptors that are involved in the modulation of metabolic processes, which can regulate levels of glucose, triglycerides, and LDL/HDL.

High plasma levels of Lp(a) and LDL (low density lipoprotein) are both independent risk factors for atherosclerosis and cardiovascular disease. The Framingham Heart study demonstrated about a 3-fold increase of cardiovascular disease and mortality when comparing those with plasma LDL levels under 100 mg/dL against those with over 200 mg/dL. [Framingham Heart Study, Canadian Journal of Cardiology 4 suppA, 5A-10A (1988)]. Lowering of total plasma cholesterol through lowering of LDL has been demonstrated with statins, which inhibit the vital cholesterol biosynthesis enzyme HMG Co Reductase, This was accompanied by significant reductions in cardiac events, and in total patient mortality. [Larosa et al, J. Amer. Med. Assoc. 1999, 282 (24): 2340-6.]

Although statins demonstrate that inhibition of endogenous cholesterol synthesis is a very potent mechanism for reduction of LDL levels, there are several other mechanisms by which LDL levels can be lowered, along with total cholesterol in plasma. For example, an increase in uptake of high density lipoprotein (HDL) by the liver will diminish the overall amount of cholesterol in circulation in the periphery, and lead to a drop in LDL levels. This can be achieved by several different methods, such as upregulation of liver receptors for HDL, and an increase of bile acid synthesis from cholesterol. It can also be achieved by reducing the uptake of cholesterol and bile acids in the ileum, which is how cholestyramine the non-absorbed bile acid sequestrant reduces LDL levels in plasma. Many of the genes involved in cholesterol catabolic, metabolic and transport processes appear to be under the control of various of the RXR-heterodimerizing nuclear hormone receptors, and it is therefore reasonable to expect that compounds which interact with one or more of these receptors may have an affect on many aspects of cholesterol biochemistry, including the lowering of plasma LDL levels.

The PPAR family of RXR-heterodimerizing nuclear hormone receptors is very important in metabolic regulation. PPARα is the target of the fibrate drugs, which can raise HDL, and marginally lower LDL, and also strongly reduce triglycerides. PPARα agonists have also been revealed to have anti-obesity effects, presumably due to their effects on lipid metabolism [Willson, U.S. Pat. No. 6,028,109]. PPARy agonists, such as Rosiglitazone and Pioglitazone are marketed for lowering blood glucose in diabetics. These compounds can also show effects on serum cholesterol and triglycerides, both of which were lowered by Troglitazone, but raised by Rosiglitazone. Although PPARβ is less well characterized than the other PPARs, it also appears to be strongly involved in metabolic regulation and there are claims that using PPARβ agonists leads to lower total cholesterol levels [Shimokawa et al., U.S. Pat. No. 6,300,364].

Retinoids, such as Accutane, also have effects on metabolism, with raised triglyceride levels being a major side effect of Accutane treatment.

Without being bound by any particular theory, all of this data taken together suggests that the RXR-heterodimerizing nuclear hormone receptors are involved in a very complicated cross-regulation of metabolism, energy utilization and cholesterol homeostasis. Therefore ligands which modulate this system in favourable ways should be useful in the treatment of metabolic diseases such as dyslipidemias and diabetes. The compounds of the current invention are phosphorus-based ligands for several of these receptors, and therefore are useful in treating metabolic disease conditions, and the diseases which arise from metabolic imbalances such as atherosclerosis.

### SUMMARY OF THE INVENTION

The invention provides aryl and heteroaryl phosphorus compounds, or pharmaceutically acceptable salts thereof, of the Formula (I): 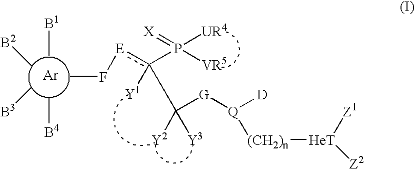
wherein
Ar is a 5-10 membered mono- or bicyclic aromatic ring containing 0-4 heteroatoms selected from the group consisting of N, O and S, with the proviso that any given ring contains a maximum of one S or one O atom;
B¹-B⁴ are independently, R¹, OR¹, NR¹R², S(O)ₙR₁, SO₂NR¹R², OCOR¹, NR¹COR², NR¹CO₂R², OCO₂R¹, NR¹CONR¹R², COR¹, CONR¹R², CH₂OR¹, CH₂NR¹R², R_{f}, OR_{f}, S(O)ₘR_{f}, CN, NO₂, F, Cl, Br, I, monocyclic aromatic,
   wherein the monocyclic aromatic is phenyl, pyridyl, pyrimidyl, pyrazinyl, pyridazinyl, thienyl, furanyl, pyrrolyl, imidazyl, pyrazolyl, oxazolyl, isoxoazolyl, thiazolyl, isothiazolyl, 1,3,4-oxadiazolyl, 1,2,4-oxadiazolyl, 1,3,4-thiadiazolyl, 1,2,3-triazolyl, 1,3,4-triazolyl, 1,2,4-triazinyl, 1,3,5-triazinyl, each optionally substituted with Z¹;
or any two of B¹-B⁴ on contiguous atoms of the aromatic ring may be taken together to form a 5-8 membered partially saturated ring (which is fused to the Ar ring) that optionally includes up to two heteroatom groups selected from NR³, O, and S(O)ₘ, and where said partially saturated ring can be optionally substituted by up to 2 groups selected independently from R³, OR³, NR³R^{3′}, F, Cl, Br, S(O)ₙR³, CN, NO₂, and ═O;
   R¹ and R² are independently H, C₁-C₈ lower alkyl, C₂-C₈ lower alkenyl, C₂-C₈ lower alkynyl, C₃-C₈ cycloalkyl, C₃-C₈ cycloalkenyl, or NR⁷R⁸, wherein R⁷ and R⁸ are independently H, C₁-C₈ lower alkyl, C₂-C₈ lower alkenyl, C₂-C₈ lower alkynyl, C₃-C₈ cycloalkyl, C₃-C₈ cycloalkenyl, or can optionally be taken together with the N to which they are bound to form a 3-8 membered azacyclic ring that optionally includes an additional heteroatom group selected from NR³, O, or S(O)ₘ, and wherein R¹ and R² are each independently optionally substituted by up to 4 groups selected from the group consisting of R³, OR³, NR³R^{3′}, F, Cl, Br, S(O)ₙR³ CN, NO₂, and =0;
   R_{f} is C₁-C₄ straight or branched lower perfluoroalkyl;
D is nothing, R³, COR³, or COR_{f};
   R³ and R³′ are independently H, C₁-C₄ lower alkyl, C₂-C₄ lower alkenyl, or C₂-C₄ alkynyl, or NR⁹R¹⁰, where R⁹ and R¹⁰ are independently H, C₁-C₄ lower alkyl, C₂-C₄ lower alkenyl, or C₂-C₄ alkynyl, or can optionally be taken together with the N to which they are bound to form a 3-8 membered azacyclic ring that optionally includes an additional heteroatom group selected from the NR³, O or S(O)ₘ, wherein R³ and R³ are optionally substituted with up to 2 groups selected from R⁶, OR⁶, SR⁶, CF₃, OCF₃, NR⁶R, =O, hydroxy, F, Cl, Br, I, CN, and NO₂;
      R⁶ and R⁶ are independently H, or C₁-C₃ lower alkyl;
E′ is a carbon;
E is nothing, or is a methine doubly bonded to E′, with the proviso that when E is a methine doubly bonded to E′, Y¹ is nothing;
F is a bond, (CH₂)ₙ, or —CH═CH—;
G is a bond or CR³R^{3′}
Het is a 5-10 membered mono- or bicyclic heteroaromatic ring containing at least one nitrogen atom;
m is 0, 1, or 2;
n is 0, 1, 2, or 3;
Q is N, CR³R³, O, S(O)ₘ, or a bond, with the proviso that when D is R³, COR³ or COR_{f}, Q is N;
R⁴ and R⁵ are each independently H, C₁-C₆ lower alkyl, C₃-C₆ lower alkenyl, C₃-C₆ lower alkynyl, C₃-C₆ lower cycloalkyl, C₄-C₆ lower cycloalkenyl, or R⁴ and R⁵ can be taken together with U, V, and the phosphorus to which they are attached, to form a 5 to 8-membered ring containing 2-7 carbon atoms, and up to 2 heteroatom groups selected from O, S(O)ₘ or NR³, wherein the 5 to 8-membered ring is optionally substituted with up to 3 substituents selected from R³, OR³, SR³, NR³R³, CF₃, OCF₃, phenyl, substituted phenyl, benzyl, substituted benzyl, ═O, and C₁-C₄ alkylidene;
U and V are each independently a bond, O, or NR³;
X is O or S; Y¹, Y², and Y³ are each independently, H, C₁-C₆ lower alkyl, C₂-C₆ lower alkenyl, C₂-C₆ lower alkynyl, C₃-C₆ lower cycloalkyl, C₄-C₆ lower cycloalkenyl, or optionally any two of Y¹, Y², and Y³ taken together with the carbons to which they are bound can form a 3-8 membered saturated ring that optionally includes one heteroatom group selected from NR³, O, and S(O)ₘ, or Y¹ and Y² taken together can form a double bond between E′ and the carbon to which Y and Y are attached, or if E is a methine doubly bonded to E′, Y¹ is nothing; and Z¹ and Z² are independently, H, C₁-C₆ lower alkyl, C₂-C₆ lower alkenyl, C₂-C₆ lower alkynyl, C₃-C₆ lower cycloalkyl, C₄-C₆ lower cycloalkenyl, OR³, NR³R³, COR³, CONR³R^{3′}, S(O)ₘR³, R_{f}, OR_{f}, S(O)ₘR_{f}, F, Cl, Br, I, CN, or NO₂;
or Z¹ and Z², when vicinally substituted, can be taken together to form a partially saturated ring of 5-7 atoms that is fused to the Het group, and optionally contains 1 or 2 heteroatoms independently selected from O, S(O)ₘ, and NR³;
with a first independent proviso that:
   when Ar is phenyl, and
   when B¹-B⁴ are each independently selected from R¹, OR¹, CH₂OR³, NR³R³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
   when D contains only single bonds, and
   when E is nothing, and
   when F is (CH₂)₀₋₂, and
   when Q is N, and
   when U, V and X are all O, and
   when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or with a second independent proviso that
   when Ar is phenyl, and
   when B¹-B⁴ are independently selected from the group R¹, OR¹, CH₂OR³, CN, F, Cl, Br, I and NO₂, or two of B¹-B⁴ on contiguous atoms of the Ar ring are taken together form a 5- or 6-membered cycloalkyl or alkylidenedioxy ring, wherein R and R³ contain only single bonds, and
   when D is nothing, and
   when E is nothing or CH, in which case Y¹ is nothing, and
   when F is (CH₂)₀₋₂ or —CH═CH—, and
   when Q is a bond, and
   when U, V and X are all O, and
   when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl.

The above first and second provisos are independent from each other and are meant to stand alone, such that only one proviso applies at any given time.

The invention also relates to compositions comprising a compound of Formula (I), or a pharmaceutically acceptable salt thereof, in combination with a pharmaceutically acceptable carrier, excipient, solvent, adjuvant or diluent.

The invention further relates to methods of regulating hypercholestemia, overall cholesterol homeostasis, LDL cholesterol levels, and/or ApoB levels in a subject comprising administering to a subject an effective amount of a compound of Formula (I), or pharmaceutically acceptable salt thereof.

The invention relates to methods of lowering serum apo (a) and Lp(a) levels in a subject comprising administering to a subject in need of such treatment an effective amount of a compound of Formula (I), or pharmaceutically acceptable salt thereof.

The invention also relates to methods of treating disease states related to high LDL levels, high ApoB levels, defective overall cholesterol homeostasis, and/or elevated apo (a) and Lp(a) levels in a mammal comprising administering to a mammal in need of such treatment a therapeutically effective amount of a compound of Formula (I), or pharmaceutically acceptable salt thereof.

The invention also relates to a method of treatment or prevention of atherosclerosis in a mammal comprising administering to a mammal in need of such treatment a therapeutically effective amount of a compound of Formula (I), or pharmaceutically acceptable salt thereof.

The invention also relates to a method of treatment or prevention of atherosclerosis in a mammal comprising coadministering to a mammal in need of such treatment a therapeutically effective amount of a compound of Formula (I), or pharmaceutically acceptable salt thereof, in combination with a compound known to reduce LDL cholesterol, such as a statin, or to increase HDL cholesterol, such as niacin or a fibrate.

The invention relates to a method of treatment or prevention of thrombosis in a mammal comprising administering to a mammal in need of such treatment an effective amount of a compound of Formula (I) or pharmaceutically acceptable salt thereof.

The invention further relates to a method of treatment or prevention of thrombosis in a mammal comprising administering to a mammal in need of such treatment an effective amount of a compound of Formula (I), or pharmaceutically acceptable salt thereof, in combination with an anti-coagulant such as Aspirin or Clopidogrel.

### DETAILED DESCRIPTION OF THE INVENTION

Definitions

Unless defined otherwise, all scientific and technical terms used herein have the same meaning as commonly understood by one of skill in the art to which this invention belongs.

All patents and publications referred to herein are hereby incorporated by reference for all purposes.

A “therapeutically effective” or “effective” amount is defined as an amount effective to reduce or lessen at least one symptom of the disease being treated or to reduce or delay onset of one or more clinical markers or symptoms of the disease.

As used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

By “alkyl” and “C₁-C₆ alkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. It is understood that in cases where an alkyl chain of a substituent (e.g. of an alkyl, alkoxy or alkenyl group) is shorter or longer than 6 carbons, it will be so indicated in the second “C” as, for example, “C₁-C₁₀” indicates a maximum of 10 carbons.

By the term “halogen” in the present invention is meant fluorine, bromine, chlorine, and iodine.

“Alkenyl” and “C₂-C₆ alkenyl” means straight and branched hydrocarbon groups having from 2 to 6 carbon atoms and from one to three double bonds and includes, for example, ethenyl, propenyl, 1-but-3-enyl, 1-pent-3-enyl, 1-hex-5-enyl and the like.

As used herein, the term “cycloalkyl” refers to saturated carbocyclic groups having three to twelve carbon atoms. The cycloalkyl can be monocyclic, or a polycyclic fused system. Examples of such groups include cyclopropyl, cyclobutyl, cyclopentyl and cyclohexyl. The cycloalkyl groups herein are unsubstituted or, as specified, substituted in one or more substitutable positions with various groups. For example, such cycloalkyl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “aryl” or “aromatic” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl), which is optionally mono-, di-, or trisubstituted. The aryl and aromatic groups herein are unsubstituted or, as specified, substituted in one or more substitutable positions with various groups. For example, such aryl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heteroaryl” is meant one or more aromatic ring systems of 5-, 6-, or 7-membered rings which includes fused ring systems of 9-11 atoms containing at least one and up to four heteroatoms selected from nitrogen, oxygen, or sulfur. Representative heteroaryl groups include pyridinyl, pyrimidinyl, quinolinyl, benzothienyl, indolyl, indolinyl, pryidazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide, in addition to those specifically recited throughout the disclosure. The heteroaryl groups herein are unsubstituted or, as specified, substituted in one or more substitutable positions with various groups. For example, such heteroaryl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heterocycle”, “heterocycloalkyl” or “heterocyclyl” is meant one or more carbocyclic ring systems of 4-, 5-, 6-, or 7-membered rings which includes fused ring systems of 9-11 atoms containing at least one and up to four heteroatoms selected from nitrogen, oxygen, or sulfur. Representative heterocycles of the invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide, homothiomorpholinyl S-oxide, in addition to those specifically recited throughout the disclosure. The heterocycle groups herein are unsubstituted or, as specified, substituted in one or more substitutable positions with various groups. For example, such heterocycle groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

In one aspect, the invention relates to compounds of Formula (I): 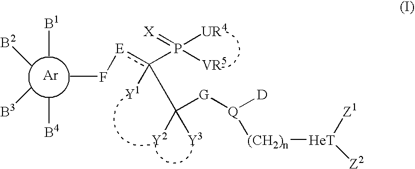
wherein
Ar is a 5-10 membered mono- or bicyclic aromatic ring containing 0-4 heteroatoms selected from the group consisting of N, O and S, with the proviso that any given ring contains a maximum of one S or one O atom;
B¹-B⁴ are independently, R¹, OR¹, NR¹R², S(O)ₙR¹, SO₂NR¹R², OCOR¹, NR¹COR², NRICO₂R², OCO₂R¹, NR¹CONR¹R², COR¹, CONR¹R², CH₂OR¹, CH₂NR¹R², R_{f}, OR_{f}, S(O)ₘR_{f}, CN, NO₂, F, Cl, Br, I, monocyclic aromatic,
   wherein the monocyclic aromatic is phenyl, pyridyl, pyrimidyl, pyrazinyl, pyridazinyl, thienyl, furanyl, pyrrolyl, imidazyl, pyrazolyl, oxazolyl, isoxoazolyl, thiazolyl, isothiazolyl, 1,3,4-oxadiazolyl, 1,2,4-oxadiazolyl, 1,3,4-thiadiazolyl, 1,2,3-triazolyl, 1,3,4-triazolyl, 1,2,4-triazinyl, 1,3,5-triazinyl, each optionally substituted with Z¹;
   or any two of B¹-B⁴ on contiguous atoms of the aromatic ring may be taken together to form a 5-8 membered partially saturated ring (which is fused to the Ar ring) that optionally includes up to two heteroatom groups selected from NR³, O, and S(O), and where said partially saturated ring can be optionally substituted by up to 2 groups selected independently from R³, OR³, NR³R³, F, Cl, Br, S(O)ₙR³ CN, NO₂, and ═O;
      R¹ and R² are independently H, C₁-C₈ lower alkyl, C₂-C₈ lower alkenyl, C₂-C₈ lower alkynyl, C₃-C₈ cycloalkyl, C₃-C₈ cycloalkenyl, or NR⁷R⁸, wherein R⁷ and R⁸ are independently H, C₁-C₈ lower alkyl, C₂-C₈ lower alkenyl, C₂-C₈ lower alkynyl, C₃-C₈ cycloalkyl, C₃-C₈ cycloalkenyl, or can optionally be taken together with the N to which they are bound to form a 3-8 membered azacyclic ring that optionally includes an additional heteroatom group selected from NR³, O, or S(O)ₘ, and wherein R¹ and R² are each independently optionally substituted by up to 4 groups selected from the group consisting of R³, OR³, NR³R³, F, Cl, Br, S(O)ₙR³ CN, NO₂, and ═O;
   R_{f} is C₁-C₄ straight or branched lower perfluoroalkyl;
D is nothing, R³, COR³, or COR_{f};
   R³ and R³′ are independently H, C₁-C₄ lower alkyl, C₂-C₄ lower alkenyl, or C₂-C₄ alkynyl, or NR⁹R¹⁰, where R⁹ and R¹⁰ are independently H, C₁-C₄ lower alkyl, C₂-C₄ lower alkenyl, or C₂-C₄ alkynyl, or can optionally be taken together with the N to which they are bound to form a 3-8 membered azacyclic ring that optionally includes an additional heteroatom group selected from the NR³, O or S(O)ₘ, wherein R³ and R³′ are optionally substituted with up to 2 groups selected from R⁶, OR⁶, SR⁶, CF₃, OCF₃, NR⁶R⁶, =O, hydroxy, F, Cl, Br, I, CN, and NO₂;
      R⁶ and R^{6′} are independently H, or C₁-C₃ lower alkyl;
E′ is a carbon;
E is nothing, or is a methine doubly bonded to E′, with the proviso that when E is a methine doubly bonded to E′, Y¹ is nothing;
F is a bond, (CH₂)ₙ, or —CH═CH—;
G is a bond or CR³R^{3′}
Het is a 5-10 membered mono- or bicyclic heteroaromatic ring containing at least one nitrogen atom;
m is 0, 1, or 2;
n is 0, 1, 2, or 3;
Q is N, CR³R^{3′}, O, S(O)ₘ, or a bond, with the proviso that when D is R³, COR³ or COR_{f}, Q is N;
R⁴ and R⁵ are each independently H, C₁-C₆ lower alkyl, C₃-C₆ lower alkenyl, C₃-C₆ lower alkynyl, C₃-C₆ lower cycloalkyl, C₄-C₆ lower cycloalkenyl, or R⁴ and R⁵ can be taken together with U, V, and the phosphorus to which they are attached, to form a 5 to 8-membered ring containing 2-7 carbon atoms, and up to 2 heteroatom groups selected from O, S(O)ₘ or NR³, wherein the 5 to 8-membered ring is optionally substituted with up to 3 substituents selected from R³, OR³, SR³, NR³R³, CF₃, OCF₃, phenyl, substituted phenyl, benzyl, substituted benzyl, ═O, and C₁-C₄ alkylidene;
U and V are each independently a bond, O, or NR³;
X is O or S; Y¹, Y², and Y³ are each independently, H, C₁-C₆ lower alkyl, C₂-C₆ lower alkenyl, C₂-C₆ lower alkynyl, C₃-C₆ lower cycloalkyl, C₄-C₆ lower cycloalkenyl, or optionally any two of Y¹, Y², and Y³ taken together with the carbons to which they are bound can form a 3-8 membered saturated ring that optionally includes one heteroatom group selected from NR³, O, and S(O)ₘ, or Y¹ and Y² taken together can form a double bond between E′ and the carbon to which Y² and Y³ are attached, or if E is a methine doubly bonded to E′, Y¹ is nothing; and
Z¹ and Z² are independently, H, C₁-C₆ lower alkyl, C₂-C₆ lower alkenyl, C₂-C₆ lower alkynyl, C₃-C₆ lower cycloalkyl, C₄-C₆ lower cycloalkenyl, OR³, NR³R³, COR³, CONR³R^{3′}, S(O)ₘR³, R_{f}, OR_{f}, S(O)ₘR_{f}, F, Cl, Br, I, CN, or NO₂;
   or Z¹ and Z², when vicinally substituted, can be taken together to form a partially saturated ring of 5-7 atoms that is fused to the Het group, and optionally contains 1 or 2 heteroatoms independently selected from O, S(O)ₘ, and NR³;
with a first independent proviso that:
   when Ar is phenyl, and
   when B¹-B⁴ are each independently selected from R¹, OR₁, CH₂OR³, NR³R³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
   when D contains only single bonds, and
   when E is nothing, and
   when F is (CH₂)₀₋₂, and
   when Q is N, and
   when U, V and X are all O, and
   when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or with a second independent proviso that
   when Ar is phenyl, and
   when B¹-B⁴ are independently selected from the group R¹, OR¹, CH₂OR³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
   when D is nothing, and
   when E is nothing or CH, in which case Y¹ is nothing, and
   when F is (CH₂)₀₋₂ or —CH═CH—, and
   when Q is a bond, and when U, V and X are all O, and
   when Y¹-Y³ are H or C₁-C₄ lower alkyl, or Y¹ and Y² taken together are a double bond and Y³ is H or C₁-C₄ lower alkyl, then one of R⁴ and R⁵ is not H or unsubstituted C₁-C₆ straight or branched chain lower alkyl;
      or a pharmaceutically acceptable salt thereof.

In one embodiment of this aspect, the compound is of Formula (I), wherein Ar is selected from phenyl, thienyl, furanyl, pyrrolyl, imidazyl, pyrazolyl, oxazolyl, isoxoazolyl, thiazolyl, isothiazolyl, benzofuranyl, benzothienyl, indolyl, indazolyl, benzimidazyl, benzoxazoyl, benzoisozazolyl, benzothiazoyl, benzoisothiazolyl, naphthyl, quinolinyl, isoquinolinyl pyrrolothienyl, furanothienyl, or thienothienyl.

In one embodiment of this aspect, the compound is of Formula (I), wherein Het is pyrrolyl, imidazyl, pyrazolyl, oxazolyl, isoxoazolyl, thiazolyl, isothiazolyl, 1,3,4-oxadiazolyl, 1,2,4-oxadiazolyl, 1,3,4-thiadiazolyl, 1,2,3-triazolyl, 1,3,4-triazolyl, pyridyl, pyrimidyl, pyrazinyl, pyridazinyl, 1,2,4-triazinyl, 1,3,5-triazinyl, pyrrolothienyl, pyrrolooxazolyl, pyrrolothiazolyl, pyrroloimidazolyl, pyrrolopyrazolyl, indolyl, indazolyl, benzimidazyl, benzoxazoyl, benzoisozazolyl, benzothiazoyl, benzoisothiazolyl, pyrrolopyridyl, thienopyridyl, furanopyridyl, pyrrolopyrimidyl, thienopyrimidyl, furanopyrimidyl, oxazolopyridyl, oxazolopyrimidyl, thiazolopyridyl, thiazolopyrimidyl, imidazolopyridyl, purinyl, quinolinyl, isoquinolinyl, quinazolinyl, quinoxalinyl, cinnolinyl, phthalazinyl, naphthyridinyl, or pyridopyrimidyl.

In another embodiment, the invention provides compounds of Formula (I): 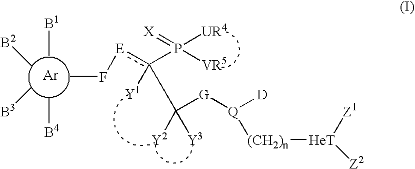
wherein
Ar is phenyl, thienyl, furanyl, pyrrolyl, imidazyl, oxazolyl, thiazolyl, benzofuranyl, benzothienyl, indolyl, benzimidazyl, benzoxazoyl, benzothiazoyl, or naphthyl;
B¹-B⁴ are independently R¹, OR¹, CH₂OR³, OCOR¹, NR¹COR¹, NR¹R³, F, Cl, Br or I, or any two of B¹-B⁴ on contiguous aromatic positions may be taken together to form a 5-7 membered partially saturated ring, which optionally includes up to two heteroatoms selected from NR³, O, and S(O)ₘ, and said ring may be optionally substituted by up to 2 groups selected independently from R³, OR³, NR³R³, F, Cl, Br, S(O)ₙ)R³, CN, NO₂, and ═O;
   R¹ and R² are independently H, C₁-C₈ lower alkyl, C₂-C₈ lower alkenyl, C₂-C₈ lower alkynyl, C₃-C₈ cycloalkyl, C₃-C₈ cycloalkenyl, or NR⁷R⁸, wherein R⁷ and R⁸ are independently H, C₁-C₈ lower alkyl, C₂-C₈ lower alkenyl, C₂-C₈ lower alkynyl, C₃-C₈ cycloalkyl, C₃-C₈ cycloalkenyl, or can optionally be taken together with the N to which they are bound to form a 3-8 membered azacyclic ring that optionally includes an additional heteroatom group selected from NR³, O, or S(O)ₘ, and wherein R¹ and R² are each independently optionally substituted by up to 4 groups selected from the group consisting of R³, OR³, NR³R^{3′}, F, Cl, Br, S(O)NR³, CN, NO₂, and ═O;
D is R³;
   R³ and R³ are independently H, C₁-C₄ lower alkyl, C₂-C₄ lower alkenyl, or C₂-C₄ alkynyl, or NR⁹R¹⁰, where R⁹ and R¹⁰ are independently H, C₁-C₄ lower alkyl, C₂-C₄ lower alkenyl, or C₂-C₄ alkynyl, or can be taken together with the N to which they are bound to form a 3-8 membered azacyclic ring that optionally includes an additional heteroatom group selected from the NR³, O or S(O)ₘ, wherein R³ and R³ are optionally substituted with up to 2 groups selected from R⁶, OR⁶, SR⁶, CF₃, OCF₃, NR⁶R⁶, ═O, hydroxy, F, Cl, Br, I, CN, and NO₂;
   R⁶ and R⁶ are independently H, or C₁-C₃ lower alkyl;
E′ is a carbon;
E is nothing, or is a methine doubly bonded to E′, with the proviso that when E is a methine doubly bonded to E′, Y¹ is nothing;
F is a bond;
G is a bond or CR³₂;
Het is pyridyl, pyrazinyl, pyrimidyl, pyridazinyl, imidazyl, oxazolyl, thiazolyl, or 1,2,4-triazinyl;
m is 0, 1 or 2;
n is 0 or 1;
Q is N, CR³R^{3′}, or O;
R⁴ and R⁵ are each independently C₁-C₆ lower alkyl, C₃-C₆ lower alkenyl, C₃-C₆ lower alkynyl, C₃-C₆ lower cycloalkyl, C₄-C₆ lower cycloalkenyl, or R⁴ and R⁵ can be taken together with U, V, and the phosphorus to which they are attached, to form a 5 to 8-membered ring containing 2-7 carbon atoms, and up to 2 heteroatoms selected from O, S(O)ₘ or NR³, wherein the 5 to 8-membered ring is optionally substituted with up to 3 substituents selected from R³, OR³, SR³, NR³R³;
U and V are each independently O or NR³;
X is O;
Y¹, Y², and Y³ are each independently H, C₁-C₆ lower alkyl, C₂-C₆ lower alkenyl, C₂-C₆ lower alkynyl, C₃-C₆ lower cycloalkyl, C₄-C₆ lower cycloalkenyl, or Y¹ and Y² taken together can form a double bond betweem E′ and the carbon to which Y² and Y³ are attached, or if E is a methine doubly bonded to E′, Y¹ is nothing; and
Z¹ and Z² are independently lone pair, H, C₁-C₆ lower alkyl, C₂-C₆ lower alkenyl, C₂-C₆ lower alkynyl, C₃-C₆ lower cycloalkyl, C₄-C₆ lower cycloalkenyl, OR³, NR R³, COR³, CONR³R³, S(O)ₘR³, R_{f}, OR_{f}, S(O)ₙR_{f}, F, Cl, Br, I, CN NO₂;
or Z¹ and Z², when vicinally substituted, can be taken together to form a partially saturated ring of 5-7 atoms that is fused to the Het group, and optionally contains 1 or 2 heteroatoms independently selected from O, S(O)ₘ, and NR³;
with a first independent proviso that:
   when Ar is phenyl, and
   when B¹-B⁴ are each independently selected from R¹, OR¹, CH₂OR³, NR³R³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
   when D contains only single bonds, and
   when E is nothing, and
   when F is (CH₂)₀₋₂, and
   when Q is N, and
   when U, V and X are all O, and
   when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or with a second independent proviso that
   when Ar is phenyl, and
   when B¹-B⁴ are independently selected from the group R¹, OR₁, CH₂OR³, CN, F, Cl, Br, I and NO₂, or two of B¹-B⁴ on contiguous atoms of the Ar ring are taken together form a 5 or 6-membered cycloalkyl or alkylidenedioxy ring, wherein R¹ and R³ contain only single bonds, and
   when D is nothing, and
   when E is nothing or CH, in which case Y¹ is nothing, and
   when F is (CH₂)₀₋₂ or —CH═CH—, and
   when Q is a bond, and
   when U, V and X are all O, and
   when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
   or a pharmaceutically acceptable salt thereof.

In another embodiment, the invention provides compounds of Formula (I): 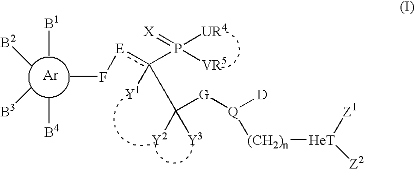
wherein
Ar is phenyl, thienyl, imidazyl, oxazolyl or thiazolyl;
   B¹-B⁴ are independently R¹, OR¹, CH₂OR³, OCOR¹, NR¹COR², NR¹R³, F, Cl, Br or I,
D is R³;
   R¹ and R² are independently H, C₁-C₈ lower alkyl, C₂-C₈ lower alkenyl, C₂-C₈ lower alkynyl, C₃-C₈ cycloalkyl, C₃-C₈ cycloalkenyl, or NR⁷R⁸, wherein R⁷ and R⁸ are independently H, C₁-C₈ lower alkyl, C₂-C₈ lower alkenyl, C₂-C₈ lower alkynyl, C₃-C₈ cycloalkyl, C₃-C₈ cycloalkenyl, or can optionally be taken together with the N to which they are bound to form a 3-8 membered azacyclic ring that optionally includes an additional heteroatom group selected from NR³, O, or S(O)ₘ, and wherein R¹ and R² are each independently optionally substituted by up to 4 groups selected from the group consisting of R³, OR³, NR³R³, F, Cl, Br, S(O)ₙR³, CN, NO₂, and ═O;
   R³ and R³′ are independently H, C₁-C₄ lower alkyl, C₂-C₄ lower alkenyl, or C₂-C₄ alkynyl, or NR⁹R¹⁰, where R⁹ and R¹⁰ are independently H, C₁-C₄ lower alkyl, C₂-C₄ lower alkenyl, or C₂-C₄ alkynyl, or can be taken together with the N to which they are bound to form a 3-8 membered azacyclic ring that optionally includes an additional heteroatom group selected from the NR³, O or S(O)ₘ, wherein R³ and R³ are optionally substituted with up to 2 groups selected from R⁶, OR⁶, SR, CF₃, OCF₃, NR⁶R⁶, ═O, hydroxy, F, Cl, Br, I, CN, and NO₂;
      R⁶ and R⁶ are independently H, or C₁-C₃ lower alkyl;
E′ is a carbon;
E is nothing;
F is a bond;
G is a bond or CR³R^{3′};
Het is pyridyl, pyrazinyl, pyrimidyl, pyridazinyl, imidazyl, oxazolyl, thiazolyl or 1,2,4-triazinyl
m is 0, 1 or 2;
n is 0;
Q is N, CR³R^{3′}, or O;
R⁴ and R⁵ are each independently C₁-C₆ lower alkyl, C₃-C₆ lower alkenyl, C₃-C₆ lower alkynyl, C₃-C₆ lower cycloalkyl, C₄-C₆ lower cycloalkenyl, or R⁴ and R⁵ can be taken together with U, V, and the phosphorus to which they are attached, to form a 5 to 8-membered ring containing 2-7 carbon atoms, and up to 2 heteroatoms selected from O, S(O)ₘ or NR³, wherein the 5 to 8-membered ring is optionally substituted with up to 3 substituents selected from R³, OR³, SR³, NR³R³;
U and V are each independently O or NR³;
X is O;
Y¹-Y², and Y³ are each independently H, C₁-C₆ lower alkyl, C₂-C₆ lower alkenyl, C₂-C₆ lower alkynyl, or Y¹ and Y² taken together can form a double bond between E′ and the carbon to which Y² and Y³ are attached; and
Z¹ and Z² are independently lone pair, H, C₁-C₆ lower alkyl, OR³, NR³R³, COR³, CONR³R³, S(O)ₘR³, R_{f}, OR_{f}, S(O)ₘR_{f}, F, Cl, Br, I;
with a first independent proviso that:
   when Ar is phenyl, and
   when B¹-B⁴ are each independently selected from R¹, OR₁, CH₂OR³, NR³R³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
   when D contains only single bonds, and
   when E is nothing, and
   when F is (CH₂)₀₋₂, and
   when Q is N, and
   when U, V and X are all O, and
   when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or with a second independent proviso that
   when Ar is phenyl, and
   when B¹-B⁴ are independently selected from the group R¹, OR¹, CH₂OR³, CN, F, Cl, Br, I and NO₂, or two of B¹-B⁴ on contiguous atoms of the Ar ring are taken together form a 5 or 6-membered cycloalkyl or alkylidenedioxy ring, wherein R¹ and R³ contain only single bonds, and
   when D is nothing, and
   when E is nothing or CH, in which case Y¹ is nothing, and
   when F is (CH₂)₀₋₂ or —CH═CH—, and
   when Q is a bond, and
   when U, V and X are all O, and
   when Y¹-Y³ are H or C₁-C₄ lower alkyl, or Y¹ and Y² taken together are a double bond and Y³ is H or C₁-C₄ lower alkyl,
   then one of R⁴ and R⁵ is not H or unsubstituted C₁-C₆ straight or branched chain lower alkyl;
      or a pharmaceutically acceptable salt thereof.

In a preferred embodiment, the compound of Formula (I) is selected from:
2,6-dimethyl-3-(2-[diethoxyphosphinyl]-2-(5-hydroxy-4-methylimidazol-2-yl)ethylamino)pyridine;
2,6-dimethyl-3-(2-[diethoxyphosphinyl]-2-(5-[N-methylamino]thiazol-2-yl)ethylamino)pyridine;
2,6-dimethyl-3-(2-[diethoxyphosphinyl]-2-[5-(4-hydoxyphenyl)thien-2-yl]ethylamino)pyridine;
2,6-dimethyl-3-(2-[diethoxyphosphinyl]-2-[6-methoxybenzothien-2-yl]ethylamino)pyridine;
2,6-dimethyl-3-(2-[diethoxythiophosphinyl]-2-(5-hydroxy-4-methylimidazol-2-yl)ethylamino)pyridine;
2,6-dimethyl-3-(2-[diethoxythiophosphinyl]-2-(5-[N-methylamino]thiazol-2-yl)ethylamino)pyridine;
2,6-dimethyl-3-(2-[diethoxythiophosphinyl]-2-[5-(4-hydoxyphenyl)thien-2-yl]ethylamino)pyridine;
2,6-dimethyl-3-(2-[diethoxythiophosphinyl]-2-[6-methoxybenzothien-2-yl]ethylamino)pyridine;
2,6-dimethyl-3-(2-[1N,3N-dimethyl-2-oxo-1,3,2-diazaphosphinan-2-yl]-2-(5-hydroxy-4-methylimidazol-2-yl)ethylamino)pyridine;
2,6-dimethyl-3-(2-[1N,3N-dimethyl-2-oxo-1,3,2-diazaphosphinan-2-yl]-2-(5-[N-methylamino]thiazol-2-yl)ethylamino)pyridine;
2,6-dimethyl-3-(2-[1N,3N-dimethyl-2-oxo-1,3,2-diazaphosphinan-2-yl]-2-[5-(4-hydoxyphenyl)thien-2-yl]ethylamino)pyridine;
2,6-dimethyl-3-(2-[1N,3N-dimethyl-2-oxo-1,3,2-diazaphosphinan-2-yl]-2-[6-methoxybenzothien-2-yl]ethylamino)pyridine;
2,6-dimethyl-3-(2-[[4S,5R]-dimethyl-2-oxo-1,3,2-diazaphospholidin-2-yl]-2-(5-hydroxy-4-methylimidazol-2-yl)ethylamino)pyridine;
2,6-dimethyl-3-(2-[[4S,5R]-dimethyl-2-oxo-1,3,2-diazaphospholidin-2-yl]-2-(5-[N-methylamino]thiazol-2-yl)ethylamino)pyridine;
2,6-dimethyl-3-(2-[[4S,5R]-dimethyl-2-oxo-1,3,2-diazaphospholidin-2-yl]-2-[5-(4-hydoxyphenyl)thien-2-yl]ethylamino)pyridine;
2,6-dimethyl-3-(2-[[4S,5R]-dimethyl-2-oxo-1,3,2-diazaphospholidin-2-yl]-2-[6-methoxybenzothien-2-yl]ethylamino)pyridine;
2,6-dimethyl-3-(2-[bis-P-(N-ethylamino)phosphinyl]-2-(5-hydroxy-4-methylimidazol-2-yl)ethylamino)pyridine;
2,6-dimethyl-3-(2-[bis-P-(N-ethylamino)phosphinyl]-2-(5-[N-methylamino]thiazol-2-yl)ethylamino)pyridine;
2,6-dimethyl-3-(2-[bis-P-(N-ethylamino)phosphinyl]-2-[5-(4-hydoxyphenyl)thien-2-yl]ethylamino)pyridine;
2,6-dimethyl-3-(2-[bis-P-(N-ethylamino)phosphinyl]-2-[6-methoxybenzothien-2-yl]ethylamino)pyridine;
2,6-dimethyl-3-(2-[2-oxo-1,3,2-dioxaphospholidin-2-yl]-2-(4-hydroxy-3-methylphenyl)ethylamino)pyridine;
2,6-dimethyl-3-(2-[1-thionoophospholidin-1-yl]-2-(4-hydroxy-3-methylphenyl)ethylamino)pyridine;
2,6-dimethyl-3-(2-[5,5-dimethyl-2-oxo-1,3,2-dioxaphosphinan-2-yl]-2-(4-hydroxy-3-methylphenyl)ethylamino)pyridine;
2,6-dimethyl-3-(3-[[4S,5R]-dimethyl-2-oxo-1,3,2-dioxaphospholidin-2-yl]-3-(4-hydroxy-3-methylphenyl)prop-1-yl)pyridine;
2,6-dimethyl-3-(2-[2-oxo-1,2-oxaphospholidin-2-yl]-3-(4-hydroxy-3-methylphenyl)prop-1-ylamino)pyridine;
2,6-dimethyl-3-(2-[5,5-dimethyl-2-oxo-1,2-oxaphosphinan-2-yl]-3-(4-hydroxy-3-methylphenyl)prop-2-en-1-ylamino)pyridine;
2,6-dimethyl-3-(2-[1-oxophosphinan-1-yl]-2-(4-hydroxy-3-methoxyphenyl)ethylamino)pyridine;
2,6-dimethyl-3-(2-[[3 S,4R]-dimethyl-1-oxophospholidin-1-yl]-2-(4-hydroxy-3-methoxy-5-methylphenyl)ethylamino)pyridine;
2,6-dimethyl-3-(2-[[4S,5S]-dimethyl-2-oxo-1,3,2-oxazaphospholidin-2-yl]-2-(indolin-2-on-5-yl)ethylamino)pyridine;
2,6-dimethyl-3-(2-[P-ethoxy-P-(N-ethylamino)phosphinyl]-2-[6-methoxybenzothien-2-yl]ethylamino)pyridine;
2,6-dimethyl-3-(2-[4[R]-methyl-1-thionophospholidin-1-yl]-2-(4-hydroxy-3-methoxyphenyl)ethylamino)pyridine;
2,6-dimethyl-3-(2-[1N-methyl-2-oxo-1,2-azaphosphinan-2-yl]-2-(4-hydroxy-3-methoxyphenyl)ethylamino)pyridine;
2,6-dimethyl-3-(3-[bis-[P-(N,N-dimethylamino)]phosphinyl]-3-(4-hydroxy-3-methoxyphenyl)prop-1-yl)pyridine;
2,6-dimethyl-3-(2-[4[R]-methyl-1-oxophospholidin-1-yl]-2-(4-hydroxy-3-methoxyphenyl)ethoxy)pyridine;
2,6-dimethyl-3-(2-[5,5-dimethyl-2-oxo-1,3,2-dioxaphosphinan-2-yl]-3-(4-hydroxy-3-methoxyphenyl)prop-2-en-1-ylamino)pyridine;
2,6-dimethyl-3-(2-[[4S,5R]-dimethyl-2-oxo-1,3,2-dioxaphospholidin-2-yl]-2-(4-hydroxy-3-methoxyphenyl)cyclohex-1-ylamino)pyridine;
2,6-dimethyl-3-(2-[2-oxo-1,2-oxaphospholidin-2-yl]-2-(4-hydroxy-3,5-dimethylphenyl)ethylamino)pyridine;
2,6-dimethyl-3-(2-[5,5-dimethyl-2-oxo-1,2-oxaphosphinan-2-yl]-2-(5-[N-methylamino]thiazol-2-yl)ethylamino)pyridine;
2,6-dimethyl-3-(2-[1-oxophosphinan-1-yl]-2-[5-(hydroxymethylbenzimidazol-2-yl]ethylamino)pyridine;
2,6-dimethyl-3-(2-[[3S,4R]-dimethyl-1-oxophospholidin-1-yl]-2-(4-hydroxy-3-methylphenyl)ethylamino)pyridine;
2,6-dimethyl-3-(2-[4[S]-methyl-1-thionophospholidin-1-yl]-2-(4-hydroxy-3,5-dimethoxyphenyl)ethylamino)pyridine;
2,6-dimethyl-3-(2-[P-ethoxy-P-(N-ethylamino)phosphinyl 2-(4-hydroxy-3,5-dimethoxyphenyl)ethylamino)pyridine;
2,6-dimethyl-3-(3-[[4S,5S]-dimethyl-2-oxo-1,3,2-oxazaphospholidin-2-yl]-3-(4-hydroxy-3,5-dimethoxyphenyl)prop-1-yl)pyridine;
2,6-dimethyl-3-(2-[1N-methyl-2-oxo-1,2-azaphosphinan-2-yl]-2-(4-hydroxy-3,5-dimethoxyphenyl)ethoxy)pyridine;
2,6-dimethyl-3-(2-[4[S]-methyl-1-oxophospholidin-1-yl]-3-(4-hydroxy-3,5-dimethoxyphenyl)prop-2-en-1-ylamino)pyridine;
2,6-dimethyl-3-(2-[4[S]-methyl-1-oxophospholidin-1-yl]-2-(4-hydroxy-3,5-dimethoxyphenyl)cyclohex-1-ylamino)pyridine;
2,6-dimethyl-3-(2-[5,5-dimethyl-2-oxo-1,3,2-dioxaphosphinan-2-yl]-2-(5-hydroxy-4-methylimidazol-2-yl)ethylamino)pyridine;
2,6-dimethyl-3-(2-[[4S,5R]-dimethyl-2-oxo-1,3,2-dioxaphospholidin-2-yl]-2-(indolin-2-on-5-yl)ethylamino)pyridine;
2,6-dimethyl-3-(2-[2-oxo-1,2-oxaphospholidin-2-yl]-2-(4-hydroxy-3-methoxyphenyl)ethylamino)pyridine;
2,6-dimethyl-3-(2-[5,5-dimethyl-2-oxo-1,2-oxaphosphinan-2-yl]-2-(4-hydroxy-3-methoxy-5-methylphenyl)ethylamino)pyridine;
2,6-dimethyl-3-(2-[diethylphosphosphiny]-2-(4-hydroxy-3-methoxy-5-methylphenyl)ethylamino)pyridine;
2,6-dimethyl-3-(2-[[5R]-methyl-2-oxo-1,2-oxaphospholidin-2-yl]-2-(4-hydroxy-3-methoxy-5-methylphenyl)ethylamino)pyridine;
2,6-dimethyl-3-(2-[[3 S,4R]-dimethyl-1-oxophospholidin-1-yl]-2-(4-hydroxy-3-methoxy-5-methylphenyl)ethoxy)pyridine;
2,6-dimethyl-3-(2-[PP-diprop-1-ylphosphinyl]-3-(4-hydroxy-3-methoxy-5-methylphenyl)prop-1-ylamino)pyridine;
2,6-dimethyl-3-(2-[P-ethoxy-P-(N-ethylamino)phosphinyl]-2-(4-hydroxy-3-methoxy-5-methylphenyl)cyclohex-1-ylamino)pyridine;
2,6-dimethyl-3-(2-[P-ethyl-P-(N-ethylamino)phosphinyl]-2-(4-hydroxy-3,5-dimethylphenyl)ethylamino)pyridine;
2,6-dimethyl-3-(2-[bis-[P-(N,N-dimethylamino)]phosphinyl]-2-[5-(4-hydoxyphenyl)thien-2-yl]ethylamino)pyridine;
2,6-dimethyl-3-(2-[3,4-dimethyl-1-oxophospholidin-1-yl]-2-[5-(hydroxymethylbenzimidazol-2-yl]ethylamino)pyridine;
2,6-dimethyl-3-(2-[5,5-dimethyl-2-oxo-1,3,2-dioxaphosphinan-2-yl]-2-(4-hydroxy-3,5-dimethoxyphenyl)ethylamino)pyridine;
2,6-dimethyl-3-(2-[[4S,5R]-dimethyl-2-oxo-1,3,2-dioxaphospholidin-2-yl]-2-(4-hydroxy-3,5-dimethylphenyl)ethylamino)pyridine;
2,6-dimethyl-3-(2-[P-ethyl-P-ethoxyphosphinyl]-2-(4-hydroxy-3,5-dimethylphenyl)ethylamino)pyridine;
2,6-dimethyl-3-(2-[2-oxo-1,2-oxaphospholidin-2-yl]-2-(4-hydroxy-3,5-dimethylphenyl)ethylamino)pyridine;
2,6-dimethyl-3-(2-[[5R]-methyl-2-oxo-1,2-oxaphospholidin-2-yl]-2-(4-hydroxy-3,5-dimethylphenyl)ethoxy)pyridine;
2,6-dimethyl-3-(2-[1-oxophosphinan-1-yl]-3-(4-hydroxy-3,5-dimethylphenyl)prop-1-ylamino)pyridine;
2,6-dimethyl-3-(2-[P,P-diprop-1-ylphosphinyl]-2-(4-hydroxy-3,5-dimethylphenyl)cyclohex-1-ylamino)pyridine;
2,6-dimethyl-3-(2-[[4S,5S]-dimethyl-2-oxo-1,3,2-oxazaphospholidin-2-yl]-2-(5-hydroxy-4-methylimidazol-2-yl)ethylamino)pyridine;
2,6-dimethyl-3-(2-[P-ethyl-P-(N-ethylamino)phosphinyl]-2-[6-methoxybenzothien-2-yl]ethylamino)pyridine;
4-methyl-2-(2-[1N-methyl-2-oxo-1,2-azaphosphinan-2-yl]-2-(4-hydroxy-3-methoxyphenyl)ethylamino)imidazole;
4-methyl-2-(2-[1-oxophospholidin-1-yl]-2-(5-hydroxy-4-methylimidazol-2-yl)ethylamino)imidazole;
4-methyl-2-(2-[1-thionophospholidin-1-yl]-2-(5-hydroxy-4-methylimidazol-2-yl)ethylamino)imidazole;
4-methyl-2-(2-[2-oxo-1,3,2-dioxaphospholidin-2-yl]-2-(5-hydroxy-4-methylimidazol-2-yl)ethylamino)imidazole;
4-methyl-2-(3-[5,5-dimethyl-2-oxo-1,3,2-dioxaphosphinan-2-yl]-3-(5-hydroxy-4-methylimidazol-2-yl)prop-1-yl)imidazole;
4-methyl-2-(2-[P-ethyl-P-ethoxyphosphinyl]-3-(5-hydroxy-4-methylimidazol-2-yl)prop-1-ylamino)imidazole;
4-methyl-2-(2-[2-oxo-1,2-oxaphospholidin-2-yl]-3-(5-hydroxy-4-methylimidazol-2-yl)prop-2-en-1-ylamino)imidazole;
4-methyl-2-(2-[[5R]-methyl-2-oxo-1,2-oxaphospholidin-2-yl]-2-(5-[N-methylamino]thiazol-2-yl)ethylamino)imidazole;
4-methyl-2-(2-[1-oxophosphinan-1-yl]-2-[5-(4-hydoxyphenyl)thien-2-yl]ethylamino)imidazole;
4-methyl-2-(2-[PP-diprop-1-ylphosphinyl]-2-(4-hydroxy-3-methylphenyl)ethylamino)imidazole;
4-methyl-2-(2-[[4S,5S]-dimethyl-2-oxo-1,3,2-oxazaphospholidin-2-yl]-2-(4-hydroxy-3,5-dimethoxyphenyl)ethylamino)imidazole;
4-methyl-2-(2-[P-ethyl-P-(N-ethylamino)phosphinyl]-2-(5-[N-methylamino]thiazol-2-yl)ethylamino)imidazole;
4-methyl-2-(2-[4[R]-methyl-1-thionophospholidin-1-yl]-2-(5-[N-methylamino]thiazol-2-yl)ethylamino)imidazole;
4-methyl-2-(2-[bis(N-ethylamino)phosphinyl]-2-(5-[N-methylamino]thiazol-2-yl)ethylamino)imidazole;
4-methyl-2-(3-[1N-methyl-2-oxo-1,2-azaphosphinan-2-yl]-3-(5-[N-methylamino]thiazol-2-yl)prop-1-yl)imidazole;
4-methyl-2-(2-[5,5-dimethyl-2-oxo-1,3,2-dioxaphosphinan-2-yl]-3-(5-[N-methylamino]thiazol-2-yl)prop-1-ylamino)imidazole;
4-methyl-2-(2-[[4S,5R]-dimethyl-2-oxo-1,3,2-dioxaphospholidin-2-yl]-3-(5-[N-methylamino]thiazol-2-yl)prop-2-en-1-ylamino)imidazole;
4-methyl-2-(2-[2-oxo-1,2-oxaphospholidin-2-yl]-2-(indolin-2-on-5-yl)ethylamino)imidazole;
4-methyl-2-(2-[5,5-dimethyl-2-oxo-1,2-oxaphosphinan-2-yl]-2-[6-methoxybenzothien-2-yl]ethylamino)imidazole;
4-methyl-2-(2-[1-oxophosphinan-1-yl]-2-(4-hydroxy-3-methoxy-5-methylphenyl)ethylamino)imidazole;
4-methyl-2-(2-[[3 S,4R]-dimethyl-1-oxophospholidin-1-yl]-2-(5-hydroxy-4-methylimidazol-2-yl)ethylamino)imidazole;
4-methyl-2-(2-[4[S]-methyl-1-thionophospholidin-1-yl]-2-(indolin-2-on-5-yl)ethylamino)imidazole;
4-methyl-2-(2-([4S,5 S]-dimethyl-2-oxo 1,3,2-diazaphospholidin-2-yl)-2-(indolin-2-on-5-yl)ethylamino)imidazole;
4-methyl-2-(3-[P,P-diprop-1-ylphosphinyl]-3-(indolin-2-on-5-yl)prop-1-yl)imidazole;
4-methyl-2-(2-[[4S,5S]-dimethyl-2-oxo-1,3,2-oxazaphospholidin-2-yl]-2-(indolin-2-on-5-yl)ethoxy)imidazole;
4-methyl-2-(2-[P-ethyl-P-(N-ethylamino)phosphinyl]-3-(indolin-2-on-5-yl)prop-2-en-1-ylamino)imidazole;
4-methyl-2-(2-[1N-methyl-2-oxo-1,2-azaphosphinan-2-yl]-2-(indolin-2-on-5-yl)cyclohex-1-ylamino)imidazole;
4-methyl-2-(2-[5,5-dimethyl-2-oxo-1,3,2-dioxaphosphinan-2-yl]-2-[5-(hydroxymethylbenzimidazol-2-yl]ethylamino)imidazole;
4-methyl-2-(2-[[4S,5R]-dimethyl-2-oxo-1,3,2-dioxaphospholidin-2-yl]-2-(4-hydroxy-3-methylphenyl)ethylamino)imidazole;
4-methyl-2-(2-[2-oxo-1,2-oxaphospholidin-2-yl]-2-(4-hydroxy-3,5-dimethylphenyl)ethylamino)imidazole;
4-methyl-2-(2-[5,5-dimethyl-2-oxo-1,2-oxaphosphinan-2-yl]-2-(5-[N-methylamino]thiazol-2-yl)ethylamino)imidazole;
4-methyl-2-(2-[3,4-dimethyl-1-thionophospholidin-1-yl]-2-[5-(4-hydoxyphenyl)thien-2-yl]ethylamino)imidazole;
4-methyl-2-(2-([4S]-ethyl-2-oxo-1,3,2-diazaphospholidin-2-yl]-2-[5-(4-hydoxyphenyl)thien-2-yl]ethylamino)imidazole;
4-methyl-2-(3-[1-oxophosphinan-1-yl]-3-[5-(4-hydoxyphenyl)thien-2-yl]prop-1-yl)imidazole;
4-methyl-2-(2-[[3S,4R]-dimethyl-1-oxophospholidin-1-yl]-2-[5-(4-hydoxyphenyl)thien-2-yl]ethoxy)imidazole;
4-methyl-2-(2-[[4S,5S]-dimethyl-2-oxo-1,3,2-oxazaphospholidin-2-yl]-3-[5-(4-hydoxyphenyl)thien-2-yl]prop-2-en-1-ylamino)imidazole;
4-methyl-2-(2-[P-ethoxy-P-(N-ethylamino)phosphinyl]-2-[5-(4-hydoxyphenyl)thien-2-yl]cyclohex-1-ylamino)imidazole;
4-methyl-2-(2-[1N-methyl-2-oxo-1,2-azaphosphinan-2-yl]-2-(4-hydroxy-3-methoxyphenyl)ethylamino)imidazole;
4-methyl-2-(2-[bis-[P-(N,N-dimethylamino)]phosphinyl]-2-(4-hydroxy-3-methoxy-5-methylphenyl)ethylamino)imidazole;
4-methyl-2-(2-[5,5-dimethyl-2-oxo-1,3,2-dioxaphosphinan-2-yl]-2-(indolin-2-on-5-yl)ethylamino)imidazole;
4-methyl-2-(2-[[4S,5R]-dimethyl-2-oxo-1,3,2-dioxaphospholidin-2-yl]-2-[6-methoxybenzothien-2-yl]ethylamino)imidazole;
4-methyl-2-(2-[P-ethyl (N,N-dimethylamino)phosphinyl]-2-[6-methoxybenzothien-2-yl]ethylamino)imidazole;
4-methyl-2-(2-[P-ethyl (N-ethylamino)phosphinyl]-2-[6-methoxybenzothien-2-yl]ethylamino)imidazole;
4-methyl-2-(2-[2-oxo-1,2-oxaphospholidin-2-yl]-2-[6-methoxybenzothien-2-yl]ethoxy)imidazole;
4-methyl-2-(2-[5,5-dimethyl-2-oxo-1,2-oxaphosphinan-2-yl]-3-[6-methoxybenzothien-2-yl]prop-1-ylamino)imidazole;
4-methyl-2-(2-[1-oxophosphinan-1-yl]-2-[6-methoxybenzothien-2-yl]cyclohex-1-ylamino)imidazole;
4-methyl-2-(2-[[3S,4R]-dimethyl-1-oxophospholidin-1-yl]-2-[5-(hydroxymethylbenzimidazol-2-yl]ethylamino)imidazole;
4,6-dimethyl-2-(2-[P,P-diprop-1-ylphosphinyl]-2-(4-hydroxy-3-methylphenyl)ethylamino)pyrimidine;
4,6-dimethyl-2-(2-[P-ethoxy-P-(N-ethylamino)phosphinyl]-2-(4-hydroxy-3,5-dimethylphenyl)ethylamino)pyrimidine;
4,6-dimethyl-2-(2-[P-ethyl-P-(N-ethylamino)phosphinyl]-2-(5-[N-methylamino]thiazol-2-yl)ethylamino)ethylamino)pyrimidine;
4,6-dimethyl-2-(2-[bis-[P-(N,N-dimethylamino)]phosphinyl yl]-2-[5-(hydroxymethylbenzimidazol-2-yl]ethylamino)pyrimidine;
4,6-dimethyl-2-(2-[1-thionophospholidin-1-yl]-2-[5-(hydroxymethylbenzimidazol-2-yl]ethylamino)pyrimidine;
4,6-dimethyl-2-(2-[5,5-dimethyl-2-oxo-1,3,2-dioxaphosphinan-2-yl]-2-[5-(hydroxymethylbenzimidazol-2-yl]ethylamino)pyrimidine;
4,6-dimethyl-2-(3-[[4S,5R]-dimethyl-2-oxo-1,3,2-dioxaphospholidin-2-yl]-3-[5-(hydroxymethylbenzimidazol-2-yl]prop-1-yl)pyrimidine;
4,6-dimethyl-2-(2-[2-oxo-1,2-oxaphospholidin-2-yl]-3-[5-(hydroxymethylbenzimidazol-2-yl]prop-1-ylamino)pyrimidine;
4,6-dimethyl-2-(2-[5,5-dimethyl-2-oxo-1,2-oxaphosphinan-2-yl]-3-[5-(hydroxymethylbenzimidazol-2-yl]prop-2-en-1-ylamino)pyrimidine;
4,6-dimethyl-2-(2-[1-oxophosphinan-1-yl]-2-(4-hydroxy-3-methylphenyl)ethylamino)pyrimidine;
4,6-dimethyl-2-(2-[4[R]-methyl-1-thionophospholidin-1-yl]-2-(4-hydroxy-3-methylphenyl)ethylamino)pyrimidine;
4,6-dimethyl-2-(2-([4R]-methyl-2-oxo-1,2-azaphosphinan-2-yl)-2-(4-hydroxy-3-methylphenyl)ethylamino)pyrimidine;
4,6-dimethyl-2-(3-[[3 S,4R]-dimethyl-1-oxophospholidin-1-yl]-3-(4-hydroxy-3-methylphenyl)prop-1-yl)pyrimidine;
4,6-dimethyl-2-(2-[[4S,5S]-dimethyl-2-oxo-1,3,2-oxazaphospholidin-2-yl]-3-(4-hydroxy-3-methylphenyl)prop-1-ylamino)pyrimidine;
4,6-dimethyl-2-(2-[P-ethoxy-P-(N-ethylamino)phosphinyl]-3-(4-hydroxy-3-methylphenyl)prop-2-en-1-ylamino)pyrimidine;
4,6-dimethyl-2-(2-[1N-methyl-2-oxo-1,2-azaphosphinan-2-yl]-2-(4-hydroxy-3-methoxyphenyl)ethylamino)pyrimidine;
4,6-dimethyl-2-(2-[bis-[P-(N,N-dimethylamino)]phosphinyl]-2-(4-hydroxy-3-methoxy-5-methylphenyl)ethylamino)pyrimidine;
4,6-dimethyl-2-(2-[5,5-dimethyl-2-oxo-1,3,2-dioxaphosphinan-2-yl]-2-(indolin-2-on-5-yl)ethylamino)pyrimidine;
4,6-dimethyl-2-(2-[[4S,5R]-dimethyl-2-oxo-1,3,2-dioxaphospholidin-2-yl]-2-[6-methoxybenzothien-2-yl]ethylamino)pyrimidine;
4,6-dimethyl-2-(2-[4[S]-methyl-1-thionophospholidin-1-yl]-2-(4-hydroxy-3-methoxyphenyl)ethylamino)pyrimidine;
4,6-dimethyl-2-(2-[P-ethoxy-P-(N,N-dimethylamino)phosphinyl]-2-(4-hydroxy-3-methoxyphenyl)ethylamino)pyrimidine;
4,6-dimethyl-2-(3-[P-ethyl-P-ethoxyphosphinyl]-3-(4-hydroxy-3-methoxyphenyl)prop-1-yl)pyrimidine;
4,6-dimethyl-2-(2-[2-oxo-1,2-oxaphospholidin-2-yl]-2-(4-hydroxy-3-methoxyphenyl)ethoxy)pyrimidine;
4,6-dimethyl-2-(2-[[5R]-methyl-2-oxo-1,2-oxaphospholidin-2-yl]-3-(4-hydroxy-3-methoxyphenyl)prop-2-en-1-ylamino)pyrimidine;
4,6-dimethyl-2-(2-[1-oxophosphinan-1-yl]-2-(4-hydroxy-3-methoxyphenyl)cyclohex-1-ylamino)pyrimidine;
4,6-dimethyl-2-(2-[P,P-diprop-1-ylphosphinyl]-2-(4-hydroxy-3,5-dimethylphenyl)ethylamino)pyrimidine;
4,6-dimethyl-2-(2-[[4S,5S]-dimethyl-2-oxo-1,3,2-oxazaphospholidin-2-yl]-2-(5-[N-methylamino]thiazol-2-yl)ethylamino)ethylamino)pyrimidine;
4,6-dimethyl-2-(2-[1N-methyl-2-oxo-1,2-azaphosphinan-2-yl]-2-[5-(hydroxymethylbenzimidazol-2-yl]ethylamino)pyrimidine;
4,6-dimethyl-2-(2-[2-oxo-1,2-azaphospholidin-2-yl]-2-(4-hydroxy-3-methylphenyl)ethylamino)pyrimidine;
4,6-dimethyl-2-(2-[3,4-dimethyl-1-thionophospholidin-1-yl]-2-(4-hydroxy-3,5-dimethoxyphenyl)ethylamino)pyrimidine;
4,6-dimethyl-2-(2-([4S,5S]-dimethyl-2-oxo-1,3,2-oxazaphospholidin-2-yl)-2-(4-hydroxy-3,5-dimethoxyphenyl)ethylamino)pyrimidine;
4,6-dimethyl-2-(3-[5,5-dimethyl-2-oxo-1,3,2-dioxaphosphinan-2-yl]-3-(4-hydroxy-3,5-dimethoxyphenyl)prop-1-yl)pyrimidine;
4,6-dimethyl-2-(2-[2-oxo-1,2-azaphospholidin-2-yl]-2-(4-hydroxy-3,5-dimethoxyphenyl)ethoxy)pyrimidine;
4,6-dimethyl-2-(2-[bis-[P-(N,N-dimethylamino)]phosphinyl]-3-(4-hydroxy-3,5-dimethoxyphenyl)prop-2-en-1-ylamino)pyrimidine;
4,6-dimethyl-2-(2-[3,4-dimethyl-1-thionophospholidin-1-yl]-2-(4-hydroxy-3,5-dimethoxyphenyl)cyclohex-1-ylamino)pyrimidine;
4,6-dimethyl-2-(2-[P-ethyl-P-ethoxyphosphinyl]-2-(5-hydroxy-4-methylimidazol-2-yl)ethylamino)pyrimidine;
4,6-dimethyl-2-(2-[2-oxo-1,2-oxaphospholidin-2-yl]-2-(indolin-2-on-5-yl)ethylamino)pyrimidine;
4,6-dimethyl-2-(2-[[5R]-methyl-2-oxo-1,2-oxaphospholidin-2-yl]-2-(4-hydroxy-3-methoxyphenyl)ethylamino)pyrimidine;
4,6-dimethyl-2-(2-[1-oxophosphinan-1-yl]-2-(4-hydroxy-3-methoxy-5-methylphenyl)ethylamino)pyrimidine;
4,6-dimethyl-2-(2-([4S]-ethyl-2-oxo-1,3,2-oxazaphospholidin-2-yl)-2-(4-hydroxy-3-methoxy-5-methylphenyl)ethylamino)pyrimidine;
4,6-dimethyl-2-(2-([4R]methyl-2-oxo-1,3,2-oxazaphosphinan-2-yl)-2-(4-hydroxy-3-methoxy-5-methylphenyl)ethylamino)pyrimidine;
4,6-dimethyl-2-(2-[[3S,4R]-dimethyl-1-oxophospholidin-1-yl]-2-(4-hydroxy-3-methoxy-5-methylphenyl)ethoxy)pyrimidine;
4,6-dimethyl-2-(2-[P,P-diprop-1-ylphosphinyl]-3-(4-hydroxy-3-methoxy-5-methylphenyl)prop-1-ylamino)pyrimidine;
4,6-dimethyl-2-(2-[P-ethoxy-P-(N-ethylamino)phosphinyl]-2-(4-hydroxy-3-methoxy-5-methylphenyl)cyclohex-1-ylamino)pyrimidine;
4,6-dimethyl-2-(2-[2-oxo-1,3,2-dioxaphospholidin-2-yl]-2-(4-hydroxy-3,5-dimethylphenyl)ethylamino)pyrimidine;
4,6-dimethyl-2-(2-[[4S,5R]-dimethyl-2-oxo-1,3,2-dioxaphospholidin-2-yl]-2-[5-(4-hydoxyphenyl)thien-2-yl]ethylamino)pyrimidine;
4,6-dimethyl-2-(2-[P-ethyl-P-ethoxyphosphinyl-1-oxophosphinan-1-yl]-2-[5-(hydroxymethylbenzimidazol-2-yl]ethylamino)pyrimidine;
3-(2-[5,5-dimethyl-2-oxo-1,2-oxaphosphinan-2-yl]-2-(4-hydroxy-3,5-dimethoxyphenyl)ethylamino)quinoline;
3-(2-[[5R]-methyl-2-oxo-1,2-oxaphospholidin-2-yl]-2-(4-hydroxy-3-methoxy-5-methylphenyl)ethylamino)quinoline;
3-(2-([4R]methyl-2-oxo-1,3,2-oxazaphosphinan-2-yl)-2-(4-hydroxy-3,5-dimethylphenyl)ethylamino)quinoline;
3-(2-([4S]-ethyl-2-oxo-1,3,2-oxazaphospholidin-2-yl)-2-(4-hydroxy-3,5-dimethylphenyl)ethylamino)quinoline;
3-(3-[1-oxophosphinan-1-yl]-3-(4-hydroxy-3,5-dimethylphenyl)prop-1-yl)quinoline;
3-(2-[[3S,4R]-dimethyl-1-oxophospholidin-1-yl]-2-(4-hydroxy-3,5-dimethylphenyl)ethoxy)quinoline;
3-(2-[[4S,5S]-dimethyl-2-oxo-1,3,2-oxazaphospholidin-2-yl]-3-(4-hydroxy-3,5-dimethylphenyl)prop-2-en-1-ylamino)quinoline;
3-(2-[P-ethoxy-P-(N-ethylamino)phosphinyl]-2-(4-hydroxy-3,5-dimethylphenyl)cyclohex-1-ylamino)quinoline;
3-(2-[1N-methyl-2-oxo-1,2-azaphosphinan-2-yl]-2-(indolin-2-on-5-yl)ethylamino)quinoline;
3-(2-[2-oxo-1,3,2-dioxaphospholidin-2-yl]-2-[6-methoxybenzothien-2-yl]ethylamino)quinoline;
3-(2-[[4S,5R]-dimethyl-2-oxo-1,3,2-dioxaphospholidin-2-yl]-2-(4-hydroxy-3-methoxy-5-methylphenyl)ethylamino)quinoline;
3-(2-[4[P-ethyl-P-ethoxyphosphinyl]-2-(5-hydroxy-4-methylimidazol-2-yl)ethylamino)quinoline;
3-(2-[3,5,5-trimethyl-2-oxo-1,3,2-oxazaphosphinan-2-yl]-2-(5-hydroxy-4-methylimidazol-2-yl)ethylamino)quinoline;
3-(2-([4S,5S]-dimethyl-2-oxo-1,3,2-oxazaphospholidin-2-yl)-2-(5-hydroxy-4-methylimidazol-2-yl)ethylamino)quinoline;
3-(3-[5,5-dimethyl-2-oxo-1,2-oxaphosphinan-2-yl]-3-(5-hydroxy-4-methylimidazol-2-yl)prop-1-yl)quinoline;
3-(2-[[5R]-methyl-2-oxo-1,2-oxaphospholidin-2-yl]-2-(5-hydroxy-4-methylimidazol-2-yl)ethoxy)quinoline;
3-(2-[[3 S,4R]-dimethyl-1-oxophospholidin-1-yl]-3-(5-hydroxy-4-methylimidazol-2-yl)prop-2-en-1-ylamino)quinoline;
3-(2-[P,P-diprop-1-ylphosphinyl]-2-(5-hydroxy-4-methylimidazol-2-yl)cyclohex-1-ylamino)quinoline;
3-(2-[P-ethoxy-P-(N-ethylamino)phosphinyl]-2-[5-(4-hydoxyphenyl)thien-2-yl]ethylamino)quinoline;
3-(2-[P-ethyl-P-(N-ethylamino)phosphinyl 2-[5-(hydroxymethylbenzimidazol-2-yl]ethylamino)quinoline;
3-(2-[2-oxo-1,2-azaphospholidin-2-yl]-2-(4-hydroxy-3,5-dimethoxyphenyl)ethylamino)quinoline;
3-(2-[2-oxo-1,3,2-dioxaphospholidin-2-yl]-2-(4-hydroxy-3,5-dimethylphenyl)ethylamino)quinoline;
3-(2-[4[S]-methyl-1-thionophospholidin-1-yl]-2-(5-[N-methylamino]thiazol-2-yl)ethylamino)quinoline;
3-(2-[P-(N-ethylamino)-P-propoxyphosphinyl]-2-(5-[N-methylamino]thiazol-2-yl)ethylamino)quinoline;
3-(2-[2-oxo-1,2-oxaphospholidin-2-yl]-2-(5-[N-methylamino]thiazol-2-yl)ethylamino)quinoline;
3-(3-[4[S]-methyl-1-thionophospholidin-1-yl]-3-(5-[N-methylamino]thiazol-2-yl)prop-1-yl)quinoline;
3-(2-[2-oxo-1,2-azaphospholidin-2-yl]-3-(5-[N-methylamino]thiazol-2-yl)prop-1-ylamino)quinoline;
3-(2-[4[S]-methyl-1-thionophospholidin-1-yl]-3-(5-[N-methylamino]thiazol-2-yl)prop-2-en-1-ylamino)quinoline;
3-(2-[[4S,5R]-dimethyl-2-oxo-1,3,2-dioxaphospholidin-2-yl]-2-(indolin-2-on-5-yl)ethylamino)quinoline;
3-(2-[P-ethyl-P-ethoxyphosphinyl]-2-[6-methoxybenzothien-2-yl]ethylamino)quinoline;
3-(2-[[5R]-methyl-2-oxo-1,2-oxaphospholidin-2-yl]-2-(4-hydroxy-3-methoxy-5-methylphenyl)ethylamino)quinoline;
3-(2-[1-oxophosphinan-1-yl]-2-(5-hydroxy-4-methylimidazol-2-yl)ethylamino)quinoline;
3-(2-[3,4-dimethyl-1-thionophospholidin-1-yl]-2-(indolin-2-on-5-yl)ethylamino)quinoline;
3-(2-([4R]-methyl-2-oxo-1,2-azaphosphinan-2-yl)-2-(indolin-2-on-5-yl)ethylamino)quinoline;
3-(2-[[5S]-methyl-2-oxo-1,2-oxaphospholidin-2-yl]-2-(indolin-2-on-5-yl)ethylamino)quinoline;
3-(2-[PP-diprop-1-ylphosphinyl]-2-(indolin-2-on-5-yl)prop-1-yl)quinoline;
3-(2-[2-oxo-1,3,2-dioxaphospholidin-2-yl]-3-(indolin-2-on-5-yl)prop-1-ylamino)quinoline;
3-(2-[5,5-dimethyl-2-oxo-1,3,2-dioxaphosphinan-2-yl]-3-(indolin-2-on-5-yl)prop-2-en-1-ylamino)quinoline;
3-(2-[P-ethyl-P-ethoxyphosphinyl]-2-[5-(4-hydoxyphenyl)thien-2-yl]ethylamino)quinoline;
3-(2-[2-oxo-1,2-oxaphospholidin-2-yl]-2-[5-(hydroxymethylbenzimidazol-2-yl]ethylamino)quinoline;
3-(2-[[5R]-methyl-2-oxo-1,2-oxaphospholidin-2-yl]-2-(4-hydroxy-3,5-dimethoxyphenyl)ethylamino)quinoline;
3-(2-[1-thionphosphinan-1-yl]-2-(4-hydroxy-3,5-dimethoxyphenyl)ethylamino)quinoline;
3-(2-([3 S,4S]-dimethyl-2-oxo-1,2-azaphospholidin-2-yl)-2-(4-hydroxy-3,5-dimethoxyphenyl)ethylamino)quinoline;
3-(2-[4,5-dimethyl-2-oxo-1,2-oxaphospholidin-2-yl]-2-(4-hydroxy-3,5-dimethoxyphenyl)ethylamino)quinoline;
3-(2-[P,P-diprop-1-ylphosphinyl]-2-(4-hydroxy-3,5-dimethoxyphenyl)ethoxy)quinoline;
3-(2-[[4S,5S]-dimethyl-2-oxo-1,3,2-oxazaphospholidin-2-yl]-3-(4-hydroxy-3,5-dimethoxyphenyl)prop-2-en-1-ylamino)quinoline;
3-(2-[P-ethyl-P-(N-ethylamino)phosphinyl]-2-(4-hydroxy-3,5-dimethoxyphenyl)cyclohex-1-ylamino)quinoline;
3-(2-[1N-methyl-2-oxo-1,2-azaphosphinan-2-yl]-2-(4-hydroxy-3-methoxy-5-methylphenyl)ethylamino)quinoline;
3-(2-[2-oxo-1,3,2-dioxaphospholidin-2-yl]-2-(indolin-2-on-5-yl)ethylamino)quinoline;
3-(2-[5,5-dimethyl-2-oxo-1,3,2-dioxaphosphinan-2-yl]-2-[6-methoxybenzothien-2-yl]ethylamino)quinoline;
5-methyl-2-(2-[P-ethyl-P-ethoxyphosphinyl]-2-(4-hydroxy-3-methoxy-5-methylphenyl)ethylamino)pyrazine;
5-methyl-2-(2-[2-oxo-1,2-oxaphospholidin-2-yl]-2-(5-hydroxy-4-methylimidazol-2-yl)ethylamino)pyrazine;
5-methyl-2-(2-[1-thionophospholidin-1-yl]-2-[5-(4-hydoxyphenyl)thien-2-yl]ethylamino)pyrazine;
5-methyl-2-(2-[P-ethyl (N-ethylamino)phosphinyl]-2-[5-(4-hydoxyphenyl)thien-2-yl]ethylamino)pyrazine;
5-methyl-2-(2-[2-oxo-1,2-oxaphosphinan-2-yl]-2-[5-(4-hydoxyphenyl)thien-2-yl]ethylamino)pyrazine;
5-methyl-3-(2-[5,5-dimethyl-2-oxo-1,2-oxaphosphinan-2-yl]-3-[5-(4-hydoxyphenyl)thien-2-yl]prop-1-yl)pyrazine;
5-methyl-2-(2-[1-oxophosphinan-1-yl]-3-[5-(4-hydoxyphenyl)thien-2-yl]prop-1-ylamino)pyrazine;
5-methyl-2-(2-[[3S,4R]-dimethyl-1-oxophospholidin-1-yl]-3-[5-(4-hydoxyphenyl)thien-2-yl]prop-2-en-1-ylamino)pyrazine;
5-methyl-2-(2-[[4S,5S]-dimethyl-2-oxo-1,3,2-oxazaphospholidin-2-yl]-2-[5-(4-hydoxyphenyl)thien-2-yl]ethylamino)pyrazine;
5-methyl-2-(2-[P-ethoxy-P-(N-ethylamino)phosphinyl]-2-[6-methoxybenzothien-2-yl]ethylamino)pyrazine;
5-methyl-2-(2-[2-oxo-1,3,2-dioxaphospholidin-2-yl]-2-(4-hydroxy-3-methoxyphenyl)ethylamino)pyrazine;
5-methyl-2-(2-[5,5-dimethyl-2-oxo-1,3,2-dioxaphosphinan-2-yl]-2-(4-hydroxy-3-methoxy-5-methylphenyl)ethylamino)pyrazine;
5-methyl-2-(2-[P-ethyl-P-ethoxyphosphinyl]-2-(indolin-2-on-5-yl)ethylamino)pyrazine;
5-methyl-2-(2-[2-oxo-1,2-oxaphospholidin-2-yl]-2-[6-methoxybenzothien-2-yl]ethylamino)pyrazine;
5-methyl-2-(2-([4R]-methyl-2-oxo-1,3,2-diazaphosphinan-2-yl)-2-[6-methoxybenzothien-2-yl]ethylamino)pyrazine;
5-methyl-2-(2-([4S]-ethyl-2-oxo-1,3,2-diazaphospholidin-2-yl)-2-[6-methoxybenzothien-2-yl]ethylamino)pyrazine;
5-methyl-3-(2-[5,5-dimethyl-2-oxo-1,2-oxaphosphinan-2-yl]-3-[6-methoxybenzothien-2-yl]prop-1-yl)pyrazine;
5-methyl-2-(2-[[5R]-methyl-2-oxo-1,2-oxaphospholidin-2-yl]-2-[6-methoxybenzothien-2-yl]ethoxy)pyrazine;
5-methyl-2-(2-[4[R]-methyl-1-oxophospholidin-1-yl]-3-[6-methoxybenzothien-2-yl]prop-2-en-1-ylamino)pyrazine;
5-methyl-2-(2-[P,P-diprop-1-ylphosphinyl]-2-[6-methoxybenzothien-2-yl]cyclohex-1-ylamino)pyrazine;
5-methyl-2-(2-[P-ethoxy-P-(N-ethylamino)phosphinyl]-2-(4-hydroxy-3-methylphenyl)ethylamino)pyrazine;
5-methyl-2-(2-[P-ethyl-P-(N-ethylamino)phosphinyl]-2-(4-hydroxy-3,5-dimethoxyphenyl)ethylamino)pyrazine;
5-methyl-2-(2-[2-oxo-1,2-azaphospholidin-2-yl]-2-(5-[N-methylamino]thiazol-2-yl)ethylamino)pyrazine;
5-methyl-2-(2-[2-oxo-1,3,2-dioxaphospholidin-2-yl]-2-[5-(4-hydoxyphenyl)thien-2-yl]ethylamino)pyrazine;
5-methyl-2-(2-[1,3,5,5-tetramethyl-2-oxo-1,3,2-diazaphosphinan-2-yl]-2-[5-(hydroxymethylbenzimidazol-2-yl]ethylamino)pyrazine;
5-methyl-2-(2-([4S,5S]-dimethyl-2-oxo 1,3,2-diazaphospholidin-2-yl)-2-[5-(hydroxymethylbenzimidazol-2-yl]ethylamino)pyrazine;
5-methyl-3-(2-[[4S,5R]-dimethyl-2-oxo-1,3,2-dioxaphospholidin-2-yl]-3-[5-(hydroxymethylbenzimidazol-2-yl]prop-1-yl)pyrazine;
5-methyl-2-(2-[P-ethyl-P-ethoxyphosphinyl]-2-[5-(hydroxymethylbenzimidazol-2-yl]ethoxy)pyrazine;
5-methyl-2-(2-[5,5-dimethyl-2-oxo-1,2-oxaphosphinan-2-yl]-3-[5-(hydroxymethylbenzimidazol-2-yl]prop-2-en-1-ylamino)pyrazine;
5-methyl-2-(2-[[5R]-methyl-2-oxo-1,2-oxaphospholidin-2-yl]-2-[5-(hydroxymethylbenzimidazol-2-yl]cyclohex-1-ylamino)pyrazine;
5-methyl-2-(2-[3,4-dimethyl-1-thionophospholidin-1-yl]-2-(4-hydroxy-3-methylphenyl)ethylamino)pyrazine;
5-methyl-2-(2-[bis(N-ethylamino)phosphinyl]-2-(4-hydroxy-3-methylphenyl)ethylamino)pyrazine;
5-methyl-2-(2-[4[R]-methyl-1-oxophospholidin-1-yl]-2-(4-hydroxy-3-methylphenyl)ethylamino)pyrazine;
5-methyl-3-(2-[P,P-diprop-1-ylphosphinyl]-3-(4-hydroxy-3-methylphenyl)prop-1-yl)pyrazine;
5-methyl-2-(2-[P-ethoxy-P-(N-ethylamino)phosphinyl]-3-(4-hydroxy-3-methylphenyl)prop-1-ylamino)pyrazine;
5-methyl-2-(2-[P-ethyl-P-(N-ethylamino)phosphinyl]-3-(4-hydroxy-3-methylphenyl)prop-2-en-1-ylamino)pyrazine;
5-methyl-2-(2-[5,5-dimethyl-2-oxo-1,3,2-dioxaphosphinan-2-yl]-2-(4-hydroxy-3-methoxyphenyl)ethylamino)pyrazine;
5-methyl-2-(2-[[4S,5R]-dimethyl-2-oxo-1,3,2-dioxaphospholidin-2-yl]-2-(4-hydroxy-3-methoxy-5-methylphenyl)ethylamino)pyrazine;
5-methyl-2-(2-[2-oxo-1,2-oxaphospholidin-2-yl]-2-(indolin-2-on-5-yl)ethylamino)pyrazine;
5-methyl-2-(2-[5,5-dimethyl-2-oxo-1,2-oxaphosphinan-2-yl]-2-[5-(hydroxymethylbenzimidazol-2-yl]ethylamino)pyrazine;
5-methyl-2-(2-[1-thionophosphinan-1-yl]-2-(4-hydroxy-3-methoxyphenyl)ethylamino)pyrazine;
5-methyl-2-(2-[1-oxophosphinan-1-yl]-2-(4-hydroxy-3-methoxyphenyl)ethylamino)pyrazine;
5-methyl-2-(2-[P,P-diprop-1-ylphosphinyl]-2-(4-hydroxy-3-methoxyphenyl)ethylamino)pyrazine;
5-methyl-3-(2-[[4S,5S]-dimethyl-2-oxo-1,3,2-oxazaphospholidin-2-yl]-3-(4-hydroxy-3-methoxyphenyl)prop-1-yl)pyrazine;
5-methyl-2-(2-[P-ethyl-P-(N-ethylamino)phosphinyl]-3-(4-hydroxy-3-methoxyphenyl)prop-1-ylamino)pyrazine;
5-methyl-2-(2-[1N-methyl-2-oxo-1,2-azaphosphinan-2-yl]-3-(4-hydroxy-3-methoxyphenyl)prop-2-en-1-ylamino)pyrazine;
5-methyl-2-(2-[1N,3N-dimethyl-2-oxo-1,3,2-diazaphosphinan-2-yl]-2-(4-hydroxy-3,5-dimethoxyphenyl)ethylamino)pyrazine;
5-methyl-2-(2-[1-oxophosphinan-1-yl]-2-(4-hydroxy-3,5-dimethylphenyl)ethylamino)pyrazine;
5-methyl-2-(2-[1N,3N-dimethyl-2-oxo-1,3,2-diazaphosphinan-2-yl]-2-[5-(4-hydoxyphenyl)thien-2-yl]ethylamino)pyrazine;
5-methyl-2-(2-[1-oxophosphinan-1-yl]-2-[5-(hydroxymethylbenzimidazol-2-yl]ethylamino)pyrazine;
2,6-dimethyl-3-(2-[2-oxo-1,3,2-dioxaphospholidin-2-yl]-2-(4-hydroxy-3-methoxy-5-methylphenyl)ethylamino)pyridine;
2,6-dimethyl-3-(2-[5,5-dimethyl-2-oxo-1,3,2-dioxaphosphinan-2-yl]-2-(4-hydroxy-3-methoxy-5-methylphenyl)ethylamino)pyridine;
2,6-dimethyl-3-(2-([4S,5S]-dimethyl-2-oxo-1,3,2-oxazaphospholidin-2-yl)-2-(4-hydroxy-3-methoxy-5-methylphenyl)ethylamino)pyridine;
2,6-dimethyl-3-(2-[3N,5,5-trimethyl-2-oxo-1,3,2-oxazaphosphinan-2-yl]-2-(4-hydroxy-3-methoxy-5-methylphenyl)ethylamino)pyridine;
2,6-dimethyl-3-(2-[5,5-dimethyl-2-oxo-1,3,2-diazaphosphinan-2-yl]-2-(4-hydroxy-3-methoxy-5-methylphenyl)ethylamino)pyridine;
2,6-dimethyl-3-(2-[bis-P-(N,N-dimethylamino)phosphinyl]-2-(4-hydroxy-3-methoxy-5-methylphenyl)ethylamino)pyridine;
2,6-dimethyl-3-(2-[bis-P-(N-ethylamino)phosphinyl]-2-(4-hydroxy-3-methoxy-5-methylphenyl)ethylamino)pyridine;
2,6-dimethyl-3-(2-[P-ethoxy-P-(N-ethylamino)phosphinyl]-2-(4-hydroxy-3-methoxy-5-methylphenyl)ethylamino)pyridine;
3,5-dimethyl-2-(2-[2-oxo-1,3,2-dioxaphospholidin-2-yl]-2-(4-hydroxy-3-methoxy-5-methylphenyl)ethylamino)pyrazine;
3,5-dimethyl-2-(2-[5,5-dimethyl-2-oxo-1,3,2-dioxaphosphinan-2-yl]-2-(4-hydroxy-3-methoxy-5-methylphenyl)ethylamino)pyrazine;
3,5-dimethyl-2-(2-([4S,5S]-dimethyl-2-oxo-1,3,2-oxazaphospholidin-2-yl)-2-(4-hydroxy-3-methoxy-5-methylphenyl)ethylamino)pyrazine;
3,5-dimethyl-2-(2-[3N,5,5-trimethyl-2-oxo-1,3,2-oxazaphosphinan-2-yl]-2-(4-hydroxy-3-methoxy-5-methylphenyl)ethylamino)pyrazine;
3,5-dimethyl-2-(2-[5,5-dimethyl-2-oxo-1,3,2-diazaphosphinan-2-yl]-2-(4-hydroxy-3-methoxy-5-methylphenyl)ethylamino)pyrazine;
3,5-dimethyl-2-(2-[bis-P-(N,N-dimethylamino)phosphinyl]-2-(4-hydroxy-3-methoxy-5-methylphenyl)ethylamino)pyrazine;
3,5-dimethyl-2-(2-[bis-P-(N-ethylamino)phosphinyl]-2-(4-hydroxy-3-methoxy-5-methylphenyl)ethylamino)pyrazine;
3,5-dimethyl-2-(2-[P-ethoxy-P-(N-ethylamino)phosphinyl]-2-(4-hydroxy-3-methoxy-5-methylphenyl)ethylamino)pyrazine;
2,4-dimethyl-5-(2-[2-oxo-1,3,2-dioxaphospholidin-2-yl]-2-(4-hydroxy-3-methoxy-5-methylphenyl)ethylamino)pyrimidine;
2,4-dimethyl-5-(2-[5,5-dimethyl-2-oxo-1,3,2-dioxaphosphinan-2-yl]-2-(4-hydroxy-3-methoxy-5-methylphenyl)ethylamino)pyrimidine;
2,4-dimethyl-5-(2-([4S,5S]-dimethyl-2-oxo-1,3,2-oxazaphospholidin-2-yl)-2-(4-hydroxy-3-methoxy-5-methylphenyl)ethylamino)pyrimidine;
2,4-dimethyl-5-(2-[3N,5,5-trimethyl-2-oxo-1,3,2-oxazaphosphinan-2-yl]-2-(4-hydroxy-3-methoxy-5-methylphenyl)ethylamino)pyrimidine;
2,4-dimethyl-5-(2-[5,5-dimethyl-2-oxo-1,3,2-diazaphosphinan-2-yl]-2-(4-hydroxy-3-methoxy-5-methylphenyl)ethylamino)pyrimidine;
2,4-dimethyl-5-(2-[bis-P-(N,N-dimethylamino)phosphinyl]-2-(4-hydroxy-3-methoxy-5-methylphenyl)ethylamino)pyrimidine;
2,4-dimethyl-5-(2-[bis-P-(N-ethylamino)phosphinyl]-2-(4-hydroxy-3-methoxy-5-methylphenyl)ethylamino)pyrimidine;
2,4-dimethyl-5-(2-[P-ethoxy-P-(N-ethylamino)phosphinyl]-2-(4-hydroxy-3-methoxy-5-methylphenyl)ethylamino)pyrimidine;
2-methyl-5-(2-[2-oxo-1,3,2-dioxaphospholidin-2-yl]-2-(4-hydroxy-3-methoxy-5-methylphenyl)ethylamino)thiazole;
2-methyl-5-(2-[5,5-dimethyl-2-oxo-1,3,2-dioxaphosphinan-2-yl]-2-(4-hydroxy-3-methoxy-5-methylphenyl)ethylamino)thiazole;
2-methyl-5-(2-([4S,5S]-dimethyl-2-oxo-1,3,2-oxazaphospholidin-2-yl)-2-(4-hydroxy-3-methoxy-5-methylphenyl)ethylamino)thiazole;
2-methyl-5-(2-[3N,5,5-trimethyl-2-oxo-1,3,2-oxazaphosphinan-2-yl]-2-(4-hydroxy-3-methoxy-5-methylphenyl)ethylamino)thiazole;
2-methyl-5-(2-[5,5-dimethyl-2-oxo-1,3,2-diazaphosphinan-2-yl]-2-(4-hydroxy-3-methoxy-5-methylphenyl)ethylamino)thiazole;
2-methyl-5-(2-[bis-P-(N,N-dimethylamino)phosphinyl]-2-(4-hydroxy-3-methoxy-5-methylphenyl)ethylamino)thiazole;
2-methyl-5-(2-[bis-P-(N-ethylamino)phosphinyl]-2-(4-hydroxy-3-methoxy-5-methylphenyl)ethylamino)thiazole; and
2-methyl-5-(2-[P-ethoxy-P-(N-ethylamino)phosphinyl]-2-(4-hydroxy-3-methoxy-5-methylphenyl)ethylamino)thiazole.

In a more preferred embodiment of this aspect, the compound of Formula (I) is selected from:
2,6-dimethyl-3-(2-[2-oxo-1,3,2-dioxaphospholidin-2-yl]-2-(4-hydroxy-3-methoxy-5-methylphenyl)ethylamino)pyridine;
2,6-dimethyl-3-(2-[5,5-dimethyl-2-oxo-1,3,2-dioxaphosphinan-2-yl]-2-(4-hydroxy-3-methoxy-5-methylphenyl)ethylamino)pyridine;
2,6-dimethyl-3-(2-([4S,5 S]-dimethyl-2-oxo-1,3,2-oxazaphospholidin-2-yl)-2-(4-hydroxy-3-methoxy-5-methylphenyl)ethylamino)pyridine;
2,6-dimethyl-3-(2-[3N,5,5-trimethyl-2-oxo-1,3,2-oxazaphosphinan-2-yl]-2-(4-hydroxy-3-methoxy-5-methylphenyl)ethylamino)pyridine;
2,6-dimethyl-3-(2-[5,5-dimethyl-2-oxo-1,3,2-diazaphosphinan-2-yl]-2-(4-hydroxy-3-methoxy-5-methylphenyl)ethylamino)pyridine;
2,6-dimethyl-3-(2-[bis-P-(N,N-dimethylamino)phosphinyl]-2-(4-hydroxy-3-methoxy-5-methylphenyl)ethylamino)pyridine;
2,6-dimethyl-3-(2-[bis-P-(N-ethylamino)phosphinyl]-2-(4-hydroxy-3-methoxy-5-methylphenyl)ethylamino)pyridine;
2,6-dimethyl-3-(2-[P-ethoxy-P-(N-ethylamino)phosphinyl]-2-(4-hydroxy-3-methoxy-5-methylphenyl)ethylamino)pyridine;
3,5-dimethyl-2-(2-[2-oxo-1,3,2-dioxaphospholidin-2-yl]-2-(4-hydroxy-3-methoxy-5-methylphenyl)ethylamino)pyrazine;
3,5-dimethyl-2-(2-[5,5-dimethyl-2-oxo-1,3,2-dioxaphosphinan-2-yl]-2-(4-hydroxy-3-methoxy-5-methylphenyl)ethylamino)pyrazine;
3,5-dimethyl-2-(2-([4S,5S]-dimethyl-2-oxo-1,3,2-oxazaphospholidin-2-yl)-2-(4-hydroxy-3-methoxy-5-methylphenyl)ethylamino)pyrazine;
3,5-dimethyl-2-(2-[3N,5,5-trimethyl-2-oxo-1,3,2-oxazaphosphinan-2-yl]-2-(4-hydroxy-3-methoxy-5-methylphenyl)ethylamino)pyrazine;
3,5-dimethyl-2-(2-[5,5-dimethyl-2-oxo-1,3,2-diazaphosphinan-2-yl]-2-(4-hydroxy-3-methoxy-5-methylphenyl)ethylamino)pyrazine;
3,5-dimethyl-2-(2-[bis-P-(N,N-dimethylamino)phosphinyl]-2-(4-hydroxy-3-methoxy-5-methylphenyl)ethylamino)pyrazine;
3,5-dimethyl-2-(2-[bis-P-(N-ethylamino)phosphinyl]-2-(4-hydroxy-3-methoxy-5-methylphenyl)ethylamino)pyrazine;
3,5-dimethyl-2-(2-[P-ethoxy-P-(N-ethylamino)phosphinyl]-2-(4-hydroxy-3-methoxy-5-methylphenyl)ethylamino)pyrazine;
2,4-dimethyl-5-(2-[2-oxo-1,3,2-dioxaphospholidin-2-yl]-2-(4-hydroxy-3-methoxy-5-methylphenyl)ethylamino)pyrimidine;
2,4-dimethyl-5-(2-[5,5-dimethyl-2-oxo-1,3,2-dioxaphosphinan-2-yl]-2-(4-hydroxy-3-methoxy-5-methylphenyl)ethylamino)pyrimidine;
2,4-dimethyl-5-(2-([4S,5S]-dimethyl-2-oxo-1,3,2-oxazaphospholidin-2-yl)-2-(4-hydroxy-3-methoxy-5-methylphenyl)ethylamino)pyrimidine;
2,4-dimethyl-5-(2-[3N,5,5-trimethyl-2-oxo-1,3,2-oxazaphosphinan-2-yl]-2-(4-hydroxy-3-methoxy-5-methylphenyl)ethylamino)pyrimidine;
2,4-dimethyl-5-(2-[5,5-dimethyl-2-oxo-1,3,2-diazaphosphinan-2-yl]-2-(4-hydroxy-3-methoxy-5-methylphenyl)ethylamino)pyrimidine;
2,4-dimethyl-5-(2-[bis-P-(N,N-dimethylamino)phosphinyl]-2-(4-hydroxy-3-methoxy-5-methylphenyl) ethyl amino)pyrimidine;
2,4-dimethyl-5-(2-[bis-P-(N-ethylamino)phosphinyl]-2-(4-hydroxy-3-methoxy-5-methylphenyl)ethylamino)pyrimidine;
2,4-dimethyl-5-(2-[P-ethoxy-P-(N-ethylamino)phosphinyl]-2-(4-hydroxy-3-methoxy-5-methylphenyl)ethylamino)pyrimidine;
2-methyl-5-(2-[2-oxo-1,3,2-dioxaphospholidin-2-yl]-2-(4-hydroxy-3-methoxy-5-methylphenyl)ethylamino)thiazole;
2-methyl-5-(2-[5,5-dimethyl-2-oxo-1,3,2-dioxaphosphinan-2-yl]-2-(4-hydroxy-3-methoxy-5-methylphenyl)ethylamino)thiazole;
2-methyl-5-(2-([4S,5 S]-dimethyl-2-oxo-1,3,2-oxazaphospholidin-2-yl)-2-(4-hydroxy-3-methoxy-5-methylphenyl)ethylamino)thiazole;
2-methyl-5-(2-[3N,5,5-trimethyl-2-oxo-1,3,2-oxazaphosphinan-2-yl]-2-(4-hydroxy-3-methoxy-5-methylphenyl)ethylamino)thiazole;
2-methyl-5-(2-[5,5-dimethyl-2-oxo-1,3,2-diazaphosphinan-2-yl]-2-(4-hydroxy-3-methoxy-5-methylphenyl)ethylamino)thiazole;
2-methyl-5-(2-[bis-P-(N,N-dimethylamino)phosphinyl]-2-(4-hydroxy-3-methoxy-5-methylphenyl)ethylamino)thiazole;
2-methyl-5-(2-[bis-P-(N-ethylamino)phosphinyl]-2-(4-hydroxy-3-methoxy-5-methylphenyl)ethylamino)thiazole; and 2-methyl-5-(2-[P-ethoxy-P-(N-ethylamino)phosphinyl]-2-(4-hydroxy-3-methoxy-5-methylphenyl)ethylamino)thiazole.

The compounds of Formula (I) may have asymmetric centers and occur as racemates, racemic mixtures and as individual diastereomers, or enantiomers. All isomeric forms are included within the scope of the invention.

In another aspect, the invention relates to pharmaceutical compositions comprising a compound, or pharmaceutically acceptable salt thereof, of Formula (I): 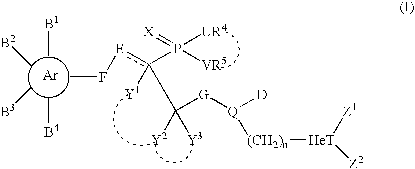
wherein
Ar is a 5-10 membered mono- or bicyclic aromatic ring containing 0-4 heteroatoms selected from the group consisting of N, O and S, with the proviso that any given ring contains a maximum of one S or one O atom;
B¹-B⁴ are independently, R¹, OR¹, NR¹R², S(O)ₙR¹, SO₂NR¹R², OCOR¹, NR¹COR², NR¹CO₂R², OCO₂R¹, NR¹CONR¹R², COR¹, CONR¹R², CH₂OR₁, CH₂NR¹R², R_{f}, OR_{f}, S(O)ₘR_{f}, CN, NO₂, F, Cl, Br, I, monocyclic aromatic,
   wherein the monocyclic aromatic is phenyl, pyridyl, pyrimidyl, pyrazinyl, pyridazinyl, thienyl, furanyl, pyrrolyl, imidazyl, pyrazolyl, oxazolyl, isoxoazolyl, thiazolyl, isothiazolyl, 1,3,4-oxadiazolyl, 1,2,4-oxadiazolyl, 1,3,4-thiadiazolyl, 1,2,3-triazolyl, 1,3,4-triazolyl, 1,2,4-triazinyl, 1,3,5-triazinyl, each optionally substituted with Z¹;
or any two of B¹-B⁴ on contiguous atoms of the aromatic ring may be taken together to form a 5-8 membered partially saturated ring (which is fused to the Ar ring) that optionally includes up to two heteroatom groups selected from NR³, O, and S(O), and where said partially saturated ring can be optionally substituted by up to 2 groups selected independently from R³, OR³, NR³R^{3′}, F, Cl, Br, S(O)ₙR³, CN, NO₂, and ═O;
   R¹ and R² are independently H, C₁-C₈ lower alkyl, C₂-C₈ lower alkenyl, C₂-C₈ lower alkynyl, C₃-C₈ cycloalkyl, C₃-C₈ cycloalkenyl, or NR⁷R⁸, wherein R⁷ and R⁸ are independently H, C₁-C₈ lower alkyl, C₂-C₈ lower alkenyl, C₂-C₈ lower alkynyl, C₃-C₈ cycloalkyl, C₃-C₈ cycloalkenyl, or can optionally be taken together with the N to which they are bound to form a 3-8 membered azacyclic ring that optionally includes an additional heteroatom group selected from NR³, O, or S(O)ₘ, and wherein R¹ and R² are each independently optionally substituted by up to 4 groups selected from the group consisting of R³, OR³, NR³R^{3′}, F, Cl, Br, S(O)ₙR³ CN, NO₂, and ═O;
   R_{f} is C₁-C₄ straight or branched lower perfluoroalkyl;
D is nothing, R³, COR³, or COR_{f};
   R³ and R³′ are independently H, C₁-C₄ lower alkyl, C₂-C₄ lower alkenyl, or C₂-C₄ alkynyl, or NR⁹R¹⁰, where R⁹ and R¹⁰ are independently H, C₁-C₄ lower alkyl, C₂-C₄ lower alkenyl, or C₂-C₄ alkynyl, or can optionally be taken together with the N to which they are bound to form a 3-8 membered azacyclic ring that optionally includes an additional heteroatom group selected from the NR³, O or S(O)ₘ, wherein R³ and R³′ are optionally substituted with up to 2 groups selected from R⁶, OR⁶, SR⁶, CF₃, OCF₃, NR⁶R^{6′}, ═O, hydroxy, F, Cl, Br, I, CN, and NO₂;
      R⁶ and R⁶′ are independently H, or C₁-C₃ lower alkyl;
E′ is a carbon;
E is nothing, or is a methine doubly bonded to E′, with the proviso that when E is a methine doubly bonded to E′, Y¹ is nothing;
F is a bond, (CH₂)ₙ, or —CH═CH—;
G is a bond or CR³R^{3′}
Het is a 5-10 membered mono- or bicyclic heteroaromatic ring containing at least one nitrogen atom;
m is 0, 1, or 2;
n is 0, 1, 2, or 3;
Q is N, CR³R^{3′}, O, S(O)ₘ, or a bond, with the proviso that when D is R³, COR³ or COR_{f}, Q is N;
R⁴ and R⁵ are each independently H, C₁-C₆ lower alkyl, C₃-C₆ lower alkenyl, C₃-C₆ lower alkynyl, C₃-C₆ lower cycloalkyl, C₄-C₆ lower cycloalkenyl, or R⁴ and R⁵ can be taken together with U, V, and the phosphorus to which they are attached, to form a 5 to 8-membered ring containing 2-7 carbon atoms, and up to 2 heteroatom groups selected from O, S(O)ₘ or NR³, wherein the 5 to 8-membered ring is optionally substituted with up to 3 substituents selected from R³, OR³, SR³, NR³R³, CF₃, OCF₃, phenyl, substituted phenyl, benzyl, substituted benzyl, ═O, and C₁-C₄ alkylidene;
U and V are each independently a bond, O, or NR³;
X is O or S;
Y¹, Y², and Y³ are each independently, H, C₁-C₆ lower alkyl, C₂-C₆ lower alkenyl, C₂-C₆ lower alkynyl, C₃-C₆ lower cycloalkyl, C₄-C₆ lower cycloalkenyl, or optionally any two of Y¹, Y², and Y³ taken together with the carbons to which they are bound can form a 3-8 membered saturated ring that optionally includes one heteroatom group selected from NR³, O, and S(O)ₘ, or Y¹ and Y² taken together can form a double bond between E′ and the carbon to which Y² and Y³ are attached, or if E is a methine doubly bonded to E′, Y¹ is nothing; and
Z¹ and Z² are independently, H, C₁-C₆ lower alkyl, C₂-C₆ lower alkenyl, C₂-C₆ lower alkynyl, C₃-C₆ lower cycloalkyl, C₄-C₆ lower cycloalkenyl, OR³, NR³R³, COR³, CoNR³R³, S(O)ₘR³, R_{f}, OR_{f}, S(O)ₘR_{f}, F, Cl, Br, I, CN, or NO₂;
   or Z¹ and Z², when vicinally substituted, can be taken together to form a partially saturated ring of 5-7 atoms that is fused to the Het group, and optionally contains 1 or 2 heteroatoms independently selected from O, S(O)ₘ, and NR³;
with a first independent proviso that:
   when Ar is phenyl, and
   when B¹-B⁴ are each independently selected from R¹, OR₁, CH₂OR³, NR³R³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
   when D contains only single bonds, and
   when E is nothing, and
   when F is (CH₂)₀₋₂, and
   when Q is N, and
   when U, V and X are all O, and
   when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or with a second independent proviso that
   when Ar is phenyl, and
   when B¹-B⁴ are independently selected from the group R¹, OR¹, CH₂OR³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
   when D is nothing, and
   when E is nothing or CH, in which case Y¹ is nothing, and
   when F is (CH₂)₀₋₂ or —CH═CH—, and
   when Q is a bond, and
   when U, V and X are all O, and
   when Y¹-Y³ are H or C₁-C₄ lower alkyl, or Y¹ and Y² taken together are a double bond and Y³ is H or C₁-C₄ lower alkyl,
      then one of R⁴ and R⁵ is not H or unsubstituted C₁-C₆ straight or branched chain lower alkyl;
         and a pharmaceutically acceptable carrier, excipient, solvent, adjuvant or diluent.

In an embodiment of this aspect, the pharmaceutical composition comprises a compound of Formula (I) selected from:
2,6-dimethyl-3-(2-[diethoxyphosphinyl]-2-(5-hydroxy-4-methylimidazol-2-yl)ethylamino)pyridine;
2,6-dimethyl-3-(2-[diethoxyphosphinyl]-2-(5-[N-methylamino]thiazol-2-yl) ethyl amino)pyri dine;
2,6-dimethyl-3-(2-[diethoxyphosphinyl]-2-[5-(4-hydoxyphenyl)thien-2-yl]ethylamino)pyridine;
2,6-dimethyl-3-(2-[diethoxyphosphinyl]-2-[6-methoxybenzothien-2-yl]ethylamino)pyridine;
2,6-dimethyl-3-(2-[diethoxythiophosphinyl]-2-(5-hydroxy-4-methylimidazol-2-yl)ethylamino)pyridine;
2,6-dimethyl-3-(2-[diethoxythiophosphinyl]-2-(5-[N-methylamino]thiazol-2-yl)ethylamino)pyridine;
2,6-dimethyl-3-(2-[diethoxythiophosphinyl]-2-[5-(4-hydoxyphenyl)thien-2-yl]ethylamino)pyridine;
2,6-dimethyl-3-(2-[diethoxythiophosphinyl]-2-[6-methoxybenzothien-2-yl]ethylamino)pyridine;
2,6-dimethyl-3-(2-[1N,3N-dimethyl-2-oxo-1,3,2-diazaphosphinan-2-yl]-2-(5-hydroxy-4-methylimidazol-2-yl)ethylamino)pyridine;
2,6-dimethyl-3-(2-[1N,3N-dimethyl-2-oxo-1,3,2-diazaphosphinan-2-yl]-2-(5-[N-methylamino]thiazol-2-yl)ethylamino)pyridine;
2,6-dimethyl-3-(2-[1N,3N-dimethyl-2-oxo-1,3,2-diazaphosphinan-2-yl]-2-[5-(4-hydoxyphenyl)thien-2-yl]ethylamino)pyridine;
2,6-dimethyl-3-(2-[1N,3N-dimethyl-2-oxo-1,3,2-diazaphosphinan-2-yl]-2-[6-methoxybenzothien-2-yl]ethylamino)pyridine;
2,6-dimethyl-3-(2-[[4S,5R]-dimethyl-2-oxo-1,3,2-diazaphospholidin-2-yl]-2-(5-hydroxy-4-methylimidazol-2-yl)ethylamino)pyridine;
2,6-dimethyl-3-(2-[[4S,5R]-dimethyl-2-oxo-1,3,2-diazaphospholidin-2-yl]-2-(5-[N-methylamino]thiazol-2-yl)ethylamino)pyridine;
2,6-dimethyl-3-(2-[[4S,5R]-dimethyl-2-oxo-1,3,2-diazaphospholidin-2-yl]-2-[5-(4-hydoxyphenyl)thien-2-yl]ethylamino)pyridine;
2,6-dimethyl-3-(2-[[4S,5R]-dimethyl-2-oxo-1,3,2-diazaphospholidin-2-yl]-2-[6-methoxybenzothien-2-yl]ethylamino)pyridine;
2,6-dimethyl-3-(2-[bis-P-(N-ethylamino)phosphinyl]-2-(5-hydroxy-4-methylimidazol-2-yl)ethylamino)pyridine;
2,6-dimethyl-3-(2-[bis-P-(N-ethylamino)phosphinyl]-2-(5-[N-methylamino]thiazol-2-yl)ethylamino)pyridine;
2,6-dimethyl-3-(2-[bis-P-(N-ethylamino)phosphinyl]-2-[5-(4-hydoxyphenyl)thien-2-yl]ethylamino)pyridine;
2,6-dimethyl-3-(2-[bis-P-(N-ethylamino)phosphinyl]-2-[6-methoxybenzothien-2-yl]ethylamino)pyridine;
2,6-dimethyl-3-(2-[2-oxo-1,3,2-dioxaphospholidin-2-yl]-2-(4-hydroxy-3-methylphenyl)ethylamino)pyridine;
2,6-dimethyl-3-(2-[1-thionoophospholidin-1-yl]-2-(4-hydroxy-3-methylphenyl)ethylamino)pyridine;
2,6-dimethyl-3-(2-[5,5-dimethyl-2-oxo-1,3,2-dioxaphosphinan-2-yl]-2-(4-hydroxy-3-methylphenyl)ethylamino)pyridine;
2,6-dimethyl-3-(3-[[4S,5R]-dimethyl-2-oxo-1,3,2-dioxaphospholidin-2-yl]-3-(4-hydroxy-3-methylphenyl)prop-1-yl)pyridine;
2,6-dimethyl-3-(2-[2-oxo-1,2-oxaphospholidin-2-yl]-3-(4-hydroxy-3-methylphenyl)prop-1-ylamino)pyridine;
2,6-dimethyl-3-(2-[5,5-dimethyl-2-oxo-1,2-oxaphosphinan-2-yl]-3-(4-hydroxy-3-methylphenyl)prop-2-en-1-ylamino)pyridine;
2,6-dimethyl-3-(2-[1-oxophosphinan-1-yl]-2-(4-hydroxy-3-methoxyphenyl)ethylamino)pyridine;
2,6-dimethyl-3-(2-[[3 S,4R]-dimethyl-1-oxophospholidin-1-yl]-2-(4-hydroxy-3-methoxy-5-methylphenyl)ethylamino)pyridine;
2,6-dimethyl-3-(2-[[4S,5S]-dimethyl-2-oxo-1,3,2-oxazaphospholidin-2-yl]-2-(indolin-2-on-5-yl)ethylamino)pyridine;
2,6-dimethyl-3-(2-[P-ethoxy-P-(N-ethylamino)phosphinyl]-2-[6-methoxybenzothien-2-yl]ethylamino)pyridine;
2,6-dimethyl-3-(2-[4[R]-methyl-1-thionophospholidin-1-yl]-2-(4-hydroxy-3-methoxyphenyl)ethylamino)pyridine;
2,6-dimethyl-3-(2-[1N-methyl-2-oxo-1,2-azaphosphinan-2-yl]-2-(4-hydroxy-3-methoxyphenyl)ethylamino)pyridine;
2,6-dimethyl-3-(3-[bis-[P-(N,N-dimethylamino)]phosphinyl]-3-(4-hydroxy-3-methoxyphenyl)prop-1-yl)pyridine;
2,6-dimethyl-3-(2-[4[R]-methyl-1-oxophospholidin-1-yl]-2-(4-hydroxy-3-methoxyphenyl)ethoxy)pyridine;
2,6-dimethyl-3-(2-[5,5-dimethyl-2-oxo-1,3,2-dioxaphosphinan-2-yl]-3-(4-hydroxy-3-methoxyphenyl)prop-2-en-1-ylamino)pyridine;
2,6-dimethyl-3-(2-[[4S,5R]-dimethyl-2-oxo-1,3,2-dioxaphospholidin-2-yl]-2-(4-hydroxy-3-methoxyphenyl)cyclohex-1-ylamino)pyridine;
2,6-dimethyl-3-(2-[2-oxo-1,2-oxaphospholidin-2-yl]-2-(4-hydroxy-3,5-dimethylphenyl)ethylamino)pyridine;
2,6-dimethyl-3-(2-[5,5-dimethyl-2-oxo-1,2-oxaphosphinan-2-yl]-2-(5-[N-methylamino]thiazol-2-yl)ethylamino)pyridine;
2,6-dimethyl-3-(2-[1-oxophosphinan-1-yl]-2-[5-(hydroxymethylbenzimidazol-2-yl]ethylamino)pyridine;
2,6-dimethyl-3-(2-[[3 S,4R]-dimethyl-1-oxophospholidin-1-yl]-2-(4-hydroxy-3-methylphenyl)ethylamino)pyridine;
2,6-dimethyl-3-(2-[4[S]-methyl-1-thionophospholidin-1-yl]-2-(4-hydroxy-3,5-dimethoxyphenyl)ethylamino)pyridine;
2,6-dimethyl-3-(2-[P-ethoxy-P-(N-ethylamino)phosphinyl 2-(4-hydroxy-3,5-dimethoxyphenyl)ethylamino)pyridine;
2,6-dimethyl-3-(3-[[4S,5S]-dimethyl-2-oxo-1,3,2-oxazaphospholidin-2-yl]-3-(4-hydroxy-3,5-dimethoxyphenyl)prop-1-yl)pyridine;
2,6-dimethyl-3-(2-[1N-methyl-2-oxo-1,2-azaphosphinan-2-yl]-2-(4-hydroxy-3,5-dimethoxyphenyl)ethoxy)pyridine;
2,6-dimethyl-3-(2-[4[S]-methyl-1-oxophospholidin-1-yl]-3-(4-hydroxy-3,5-dimethoxyphenyl)prop-2-en-1-ylamino)pyridine;
2,6-dimethyl-3-(2-[4[S]-methyl-1-oxophospholidin-1-yl]-2-(4-hydroxy-3,5-dimethoxyphenyl)cyclohex-1-ylamino)pyridine;
2,6-dimethyl-3-(2-[5,5-dimethyl-2-oxo-1,3,2-dioxaphosphinan-2-yl]-2-(5-hydroxy-4-methylimidazol-2-yl)ethylamino)pyridine;
2,6-dimethyl-3-(2-[[4S,5R]-dimethyl-2-oxo-1,3,2-dioxaphospholidin-2-yl]-2-(indolin-2-on-5-yl)ethylamino)pyridine;
2,6-dimethyl-3-(2-[2-oxo-1,2-oxaphospholidin-2-yl]-2-(4-hydroxy-3-methoxyphenyl)ethylamino)pyridine;
2,6-dimethyl-3-(2-[5,5-dimethyl-2-oxo-1,2-oxaphosphinan-2-yl]-2-(4-hydroxy-3-methoxy-5-methylphenyl)ethylamino)pyridine;
2,6-dimethyl-3-(2-[diethylphosphosphiny]-2-(4-hydroxy-3-methoxy-5-methylphenyl)ethylamino)pyridine;
2,6-dimethyl-3-(2-[[5R]-methyl-2-oxo-1,2-oxaphospholidin-2-yl]-2-(4-hydroxy-3-methoxy-5-methylphenyl)ethylamino)pyridine;
2,6-dimethyl-3-(2-[[3 S,4R]-dimethyl-1-oxophospholidin-1-yl]-2-(4-hydroxy-3-methoxy-5-methylphenyl)ethoxy)pyridine;
2,6-dimethyl-3-(2-[P,P-diprop-1-ylphosphinyl]-3-(4-hydroxy-3-methoxy-5-methylphenyl)prop-1-ylamino)pyridine;
2,6-dimethyl-3-(2-[P-ethoxy-P-(N-ethylamino)phosphinyl]-2-(4-hydroxy-3-methoxy-5-methylphenyl)cyclohex-1-ylamino)pyridine;
2,6-dimethyl-3-(2-[P-ethyl-P-(N-ethylamino)phosphinyl]-2-(4-hydroxy-3,5-dimethylphenyl)ethylamino)pyridine;
2,6-dimethyl-3-(2-[bis-[P-(N,N-dimethylamino)]phosphinyl]-2-[5-(4-hydoxyphenyl)thien-2-yl]ethylamino)pyridine;
2,6-dimethyl-3-(2-[3,4-dimethyl-1-oxophospholidin-1-yl]-2-[5-(hydroxymethylbenzimidazol-2-yl]ethylamino)pyridine;
2,6-dimethyl-3-(2-[5,5-dimethyl-2-oxo-1,3,2-dioxaphosphinan-2-yl]-2-(4-hydroxy-3,5-dimethoxyphenyl)ethylamino)pyridine;
2,6-dimethyl-3-(2-[[4S,5R]-dimethyl-2-oxo-1,3,2-dioxaphospholidin-2-yl]-2-(4-hydroxy-3,5-dimethylphenyl)ethylamino)pyridine;
2,6-dimethyl-3-(2-[P-ethyl-P-ethoxyphosphinyl]-2-(4-hydroxy-3,5-dimethylphenyl)ethylamino)pyridine;
2,6-dimethyl-3-(2-[2-oxo-1,2-oxaphospholidin-2-yl]-2-(4-hydroxy-3,5-dimethylphenyl)ethylamino)pyridine;
2,6-dimethyl-3-(2-[[5R]-methyl-2-oxo-1,2-oxaphospholidin-2-yl]-2-(4-hydroxy-3,5-dimethylphenyl)ethoxy)pyridine;
2,6-dimethyl-3-(2-[1-oxophosphinan-1-yl]-3-(4-hydroxy-3,5-dimethylphenyl)prop-1-ylamino)pyridine;
2,6-dimethyl-3-(2-[P,P-diprop-1-ylphosphinyl]-2-(4-hydroxy-3,5-dimethylphenyl)cyclohex-1-ylamino)pyridine;
2,6-dimethyl-3-(2-[[4S,5S]-dimethyl-2-oxo-1,3,2-oxazaphospholidin-2-yl]-2-(5-hydroxy-4-methylimidazol-2-yl)ethylamino)pyridine;
2,6-dimethyl-3-(2-[P-ethyl-P-(N-ethylamino)phosphinyl]-2-[6-methoxybenzothien-2-yl]ethylamino)pyridine;
4-methyl-2-(2-[1N-methyl-2-oxo-1,2-azaphosphinan-2-yl]-2-(4-hydroxy-3-methoxyphenyl)ethylamino)imidazole;
4-methyl-2-(2-[1-oxophospholidin-1-yl]-2-(5-hydroxy-4-methylimidazol-2-yl)ethylamino)imidazole;
4-methyl-2-(2-[1-thionophospholidin-1-yl]-2-(5-hydroxy-4-methylimidazol-2-yl)ethylamino)imidazole;
4-methyl-2-(2-[2-oxo-1,3,2-dioxaphospholidin-2-yl]-2-(5-hydroxy-4-methylimidazol-2-yl)ethylamino)imidazole;
4-methyl-2-(3-[5,5-dimethyl-2-oxo-1,3,2-dioxaphosphinan-2-yl]-3-(5-hydroxy-4-methylimidazol-2-yl)prop-1-yl)imidazole;
4-methyl-2-(2-[P-ethyl-P-ethoxyphosphinyl]-3-(5-hydroxy-4-methylimidazol-2-yl)prop-1-ylamino)imidazole;
4-methyl-2-(2-[2-oxo-1,2-oxaphospholidin-2-yl]-3-(5-hydroxy-4-methylimidazol-2-yl)prop-2-en-1-ylamino)imidazole;
4-methyl-2-(2-[[5R]-methyl-2-oxo-1,2-oxaphospholidin-2-yl]-2-(5-[N-methylamino]thiazol-2-yl)ethylamino)imidazole;
4-methyl-2-(2-[1-oxophosphinan-1-yl]-2-[5-(4-hydoxyphenyl)thien-2-yl]ethylamino)imidazole;
4-methyl-2-(2-[PP-diprop-1-ylphosphinyl]-2-(4-hydroxy-3-methylphenyl)ethylamino)imidazole;
4-methyl-2-(2-[[4S,5S]-dimethyl-2-oxo-1,3,2-oxazaphospholidin-2-yl]-2-(4-hydroxy-3,5-dimethoxyphenyl)ethylamino)imidazole;
4-methyl-2-(2-[P-ethyl-P-(N-ethylamino)phosphinyl]-2-(5-[N-methylamino]thiazol-2-yl)ethylamino)imidazole;
4-methyl-2-(2-[4[R]-methyl-1-thionophospholidin-1-yl]-2-(5-[N-methylamino]thiazol-2-yl)ethylamino)imidazole;
4-methyl-2-(2-[bis(N-ethylamino)phosphinyl]-2-(5-[N-methylamino]thiazol-2-yl)ethylamino)imidazole;
4-methyl-2-(3-[1N-methyl-2-oxo-1,2-azaphosphinan-2-yl]-3-(5-[N-methylamino]thiazol-2-yl)prop-1-yl)imidazole;
4-methyl-2-(2-[5,5-dimethyl-2-oxo-1,3,2-dioxaphosphinan-2-yl]-3-(5-[N-methylamino]thiazol-2-yl)prop-1-ylamino)imidazole;
4-methyl-2-(2-[[4S,5R]-dimethyl-2-oxo-1,3,2-dioxaphospholidin-2-yl]-3-(5-[N-methylamino]thiazol-2-yl)prop-2-en-1-ylamino)imidazole;
4-methyl-2-(2-[2-oxo-1,2-oxaphospholidin-2-yl]-2-(indolin-2-on-5-yl)ethylamino)imidazole;
4-methyl-2-(2-[5,5-dimethyl-2-oxo-1,2-oxaphosphinan-2-yl]-2-[6-methoxybenzothien-2-yl]ethylamino)imidazole;
4-methyl-2-(2-[1-oxophosphinan-1-yl]-2-(4-hydroxy-3-methoxy-5-methylphenyl)ethylamino)imidazole;
4-methyl-2-(2-[[3 S,4R]-dimethyl-1-oxophospholidin-1-yl]-2-(5-hydroxy-4-methylimidazol-2-yl)ethylamino)imidazole;
4-methyl-2-(2-[4[S]-methyl-1-thionophospholidin-1-yl]-2-(indolin-2-on-5-yl)ethyl amino)imidazole;
4-methyl-2-(2-([4S,5S]-dimethyl-2-oxo1,3,2-diazaphospholidin-2-yl)-2-(indolin-2-on-5-yl)ethylamino)imidazole;
4-methyl-2-(3-[P,P-diprop-1-ylphosphinyl]-3-(indolin-2-on-5-yl)prop-1-yl)imidazole;
4-methyl-2-(2-[[4S,5 S]-dimethyl-2-oxo-1,3,2-oxazaphospholidin-2-yl]-2-(indolin-2-on-5-yl)ethoxy)imidazole;
4-methyl-2-(2-[P-ethyl-P-(N-ethylamino)phosphinyl]-3-(indolin-2-on-5-yl)prop-2-en-1-ylamino)imidazole;
4-methyl-2-(2-[1N-methyl-2-oxo-1,2-azaphosphinan-2-yl]-2-(indolin-2-on-5-yl)cyclohex-1-ylamino)imidazole;
4-methyl-2-(2-[5,5-dimethyl-2-oxo-1,3,2-dioxaphosphinan-2-yl]-2-[5-(hydroxymethylbenzimidazol-2-yl]ethylamino)imidazole;
4-methyl-2-(2-[[4S,5R]-dimethyl-2-oxo-1,3,2-dioxaphospholidin-2-yl]-2-(4-hydroxy-3-methylphenyl)ethylamino)imidazole;
4-methyl-2-(2-[2-oxo-1,2-oxaphospholidin-2-yl]-2-(4-hydroxy-3,5-dimethylphenyl)ethylamino)imidazole;
4-methyl-2-(2-[5,5-dimethyl-2-oxo-1,2-oxaphosphinan-2-yl]-2-(5-[N-methylamino]thiazol-2-yl)ethylamino)imidazole;
4-methyl-2-(2-[3,4-dimethyl-1-thionophospholidin-1-yl]-2-[5-(4-hydoxyphenyl)thien-2-yl]ethylamino)imidazole;
4-methyl-2-(2-([4S]-ethyl-2-oxo-1,3,2-diazaphospholidin-2-yl]-2-[5-(4-hydoxyphenyl)thien-2-yl]ethylamino)imidazole;
4-methyl-2-(3-[1-oxophosphinan-1-yl]-3-[5-(4-hydoxyphenyl)thien-2-yl]prop-1-yl)imidazole;
4-methyl-2-(2-[[3 S,4R]-dimethyl-1-oxophospholidin-1-yl]-2-[5-(4-hydoxyphenyl)thien-2-yl]ethoxy)imidazole;
4-methyl-2-(2-[[4S,5 S]-dimethyl-2-oxo-1,3,2-oxazaphospholidin-2-yl]-3-[5-(4-hydoxyphenyl)thien-2-yl]prop-2-en-1-ylamino)imidazole;
4-methyl-2-(2-[P-ethoxy-P-(N-ethylamino)phosphinyl]-2-[5-(4-hydoxyphenyl)thien-2-yl]cyclohex-1-ylamino)imidazole;
4-methyl-2-(2-[1N-methyl-2-oxo-1,2-azaphosphinan-2-yl]-2-(4-hydroxy-3-methoxyphenyl)ethylamino)imidazole;
4-methyl-2-(2-[bis-[P-(N,N-dimethylamino)]phosphinyl]-2-(4-hydroxy-3-methoxy-5-methylphenyl)ethylamino)imidazole;
4-methyl-2-(2-[5,5-dimethyl-2-oxo-1,3,2-dioxaphosphinan-2-yl]-2-(indolin-2-on-5-yl)ethylamino)imidazole;
4-methyl-2-(2-[[4S,5R]-dimethyl-2-oxo-1,3,2-dioxaphospholidin-2-yl]-2-[6-methoxybenzothien-2-yl]ethylamino)imidazole;
4-methyl-2-(2-[P-ethyl (N,N-dimethylamino)phosphinyl]-2-[6-methoxybenzothien-2-yl]ethylamino)imidazole;
4-methyl-2-(2-[P-ethyl (N-ethylamino)phosphinyl]-2-[6-methoxybenzothien-2-yl]ethylamino)imidazole;
4-methyl-2-(2-[2-oxo-1,2-oxaphospholidin-2-yl]-2-[6-methoxybenzothien-2-yl]ethoxy)imidazole;
4-methyl-2-(2-[5,5-dimethyl-2-oxo-1,2-oxaphosphinan-2-yl]-3-[6-methoxybenzothien-2-yl]prop-1-ylamino)imidazole;
4-methyl-2-(2-[1-oxophosphinan-1-yl]-2-[6-methoxybenzothien-2-yl]cyclohex-1-ylamino)imidazole;
4-methyl-2-(2-[[3S,4R]-dimethyl-1-oxophospholidin-1-yl]-2-[5-(hydroxymethylbenzimidazol-2-yl]ethylamino)imidazole;
4,6-dimethyl-2-(2-[P,P-diprop-1-ylphosphinyl]-2-(4-hydroxy-3-methylphenyl)ethylamino)pyrimidine;
4,6-dimethyl-2-(2-[P-ethoxy-P-(N-ethylamino)phosphinyl]-2-(4-hydroxy-3,5-dimethylphenyl)ethylamino)pyrimidine;
4,6-dimethyl-2-(2-[P-ethyl-P-(N-ethylamino)phosphinyl]-2-(5-[N-methylamino]thiazol-2-yl)ethylamino)ethylamino)pyrimidine;
4,6-dimethyl-2-(2-[bis-[P-(N,N-dimethylamino)]phosphinyl yl]-2-[5-(hydroxymethylbenzimidazol-2-yl]ethylamino)pyrimidine;
4,6-dimethyl-2-(2-[1-thionophospholidin-1-yl]-2-[5-(hydroxymethylbenzimidazol-2-yl]ethylamino)pyrimidine;
4,6-dimethyl-2-(2-[5,5-dimethyl-2-oxo-1,3,2-dioxaphosphinan-2-yl]-2-[5-(hydroxymethylbenzimidazol-2-yl]ethylamino)pyrimidine;
4,6-dimethyl-2-(3-[[4S,5R]-dimethyl-2-oxo-1,3,2-dioxaphospholidin-2-yl]-3-[5-(hydroxymethylbenzimidazol-2-yl]prop-1-yl)pyrimidine;
4,6-dimethyl-2-(2-[2-oxo-1,2-oxaphospholidin-2-yl]-3-[5-(hydroxymethylbenzimidazol-2-yl]prop-1-ylamino)pyrimidine;
4,6-dimethyl-2-(2-[5,5-dimethyl-2-oxo-1,2-oxaphosphinan-2-yl]-3-[5-(hydroxymethylbenzimidazol-2-yl]prop-2-en-1-ylamino)pyrimidine;
4,6-dimethyl-2-(2-[1-oxophosphinan-1-yl]-2-(4-hydroxy-3-methylphenyl)ethylamino)pyrimidine;
4,6-dimethyl-2-(2-[4[R]-methyl-1-thionophospholidin-1-yl]-2-(4-hydroxy-3-methylphenyl)ethylamino)pyrimidine;
4,6-dimethyl-2-(2-([4R]-methyl-2-oxo-1,2-azaphosphinan-2-yl)-2-(4-hydroxy-3-methylphenyl)ethylamino)pyrimidine;
4,6-dimethyl-2-(3-[[3 S,4R]-dimethyl-1-oxophospholidin-1-yl]-3-(4-hydroxy-3-methylphenyl)prop-1-yl)pyrimidine;
4,6-dimethyl-2-(2-[[4S,5S]-dimethyl-2-oxo-1,3,2-oxazaphospholidin-2-yl]-3-(4-hydroxy-3-methylphenyl)prop-1-ylamino)pyrimidine;
4,6-dimethyl-2-(2-[P-ethoxy-P-(N-ethylamino)phosphinyl]-3-(4-hydroxy-3-methylphenyl)prop-2-en-1-ylamino)pyrimidine;
4,6-dimethyl-2-(2-[1N-methyl-2-oxo-1,2-azaphosphinan-2-yl]-2-(4-hydroxy-3-methoxyphenyl)ethylamino)pyrimidine;
4,6-dimethyl-2-(2-[bis-[P-(N,N-dimethylamino)]phosphinyl]-2-(4-hydroxy-3-methoxy-5-methylphenyl)ethylamino)pyrimidine;
4,6-dimethyl-2-(2-[5,5-dimethyl-2-oxo-1,3,2-dioxaphosphinan-2-yl]-2-(indolin-2-on-5-yl)ethylamino)pyrimidine;
4,6-dimethyl-2-(2-[[4S,5R]-dimethyl-2-oxo-1,3,2-dioxaphospholidin-2-yl]-2-[6-methoxybenzothien-2-yl]ethylamino)pyrimidine;
4,6-dimethyl-2-(2-[4 [S]-methyl-1-thionophospholidin-1-yl]-2-(4-hydroxy-3-methoxyphenyl)ethylamino)pyrimidine;
4,6-dimethyl-2-(2-[P-ethoxy-P-(N,N-dimethylamino)phosphinyl]-2-(4-hydroxy-3-methoxyphenyl)ethylamino)pyrimidine;
4,6-dimethyl-2-(3-[P-ethyl-P-ethoxyphosphinyl]-3-(4-hydroxy-3-methoxyphenyl)prop-1-yl)pyrimidine;
4,6-dimethyl-2-(2-[2-oxo-1,2-oxaphospholidin-2-yl]-2-(4-hydroxy-3-methoxyphenyl)ethoxy)pyrimidine;
4,6-dimethyl-2-(2-[[5R]-methyl-2-oxo-1,2-oxaphospholidin-2-yl]-3-(4-hydroxy-3-methoxyphenyl)prop-2-en-1-ylamino)pyrimidine;
4,6-dimethyl-2-(2-[1-oxophosphinan-1-yl]-2-(4-hydroxy-3-methoxyphenyl)cyclohex-1-ylamino)pyrimidine;
4,6-dimethyl-2-(2-[P,P-diprop-1-ylphosphinyl]-2-(4-hydroxy-3,5-dimethylphenyl)ethylamino)pyrimidine;
4,6-dimethyl-2-(2-[[4S,5S]-dimethyl-2-oxo-1,3,2-oxazaphospholidin-2-yl]-2-(5-[N-methylamino]thiazol-2-yl)ethylamino)ethylamino)pyrimidine;
4,6-dimethyl-2-(2-[1N-methyl-2-oxo-1,2-azaphosphinan-2-yl]-2-[5-(hydroxymethylbenzimidazol-2-yl]ethylamino)pyrimidine;
4,6-dimethyl-2-(2-[2-oxo-1,2-azaphospholidin-2-yl]-2-(4-hydroxy-3-methylphenyl)ethylamino)pyrimidine;
4,6-dimethyl-2-(2-[3,4-dimethyl-1-thionophospholidin-1-yl]-2-(4-hydroxy-3,5-dimethoxyphenyl)ethylamino)pyrimidine;
4,6-dimethyl-2-(2-([4S,5S]-dimethyl-2-oxo-1,3,2-oxazaphospholidin-2-yl)-2-(4-hydroxy-3,5-dimethoxyphenyl)ethylamino)pyrimidine;
4,6-dimethyl-2-(3-[5,5-dimethyl-2-oxo-1,3,2-dioxaphosphinan-2-yl]-3-(4-hydroxy-3,5-dimethoxyphenyl)prop-1-yl)pyrimidine;
4,6-dimethyl-2-(2-[2-oxo-1,2-azaphospholidin-2-yl]-2-(4-hydroxy-3,5-dimethoxyphenyl)ethoxy)pyrimidine;
4,6-dimethyl-2-(2-[bis-[P-(N,N-dimethylamino)]phosphinyl]-3-(4-hydroxy-3,5-dimethoxyphenyl)prop-2-en-1-ylamino)pyrimidine;
4,6-dimethyl-2-(2-[3,4-dimethyl-1-thionophospholidin-1-yl]-2-(4-hydroxy-3,5-dimethoxyphenyl)cyclohex-1-ylamino)pyrimidine;
4,6-dimethyl-2-(2-[P-ethyl-P-ethoxyphosphinyl]-2-(5-hydroxy-4-methylimidazol-2-yl)ethylamino)pyrimidine;
4,6-dimethyl-2-(2-[2-oxo-1,2-oxaphospholidin-2-yl]-2-(indolin-2-on-5-yl)ethylamino)pyrimidine;
4,6-dimethyl-2-(2-[[5R]-methyl-2-oxo-1,2-oxaphospholidin-2-yl]-2-(4-hydroxy-3-methoxyphenyl)ethylamino)pyrimidine;
4,6-dimethyl-2-(2-[1-oxophosphinan-1-yl]-2-(4-hydroxy-3-methoxy-5-methylphenyl)ethylamino)pyrimidine;
4,6-dimethyl-2-(2-([4S]-ethyl-2-oxo-1,3,2-oxazaphospholidin-2-yl)-2-(4-hydroxy-3-methoxy-5-methylphenyl)ethylamino)pyrimidine;
4,6-dimethyl-2-(2-([4R]methyl-2-oxo-1,3,2-oxazaphosphinan-2-yl)-2-(4-hydroxy-3-methoxy-5-methylphenyl)ethylamino)pyrimidine;
4,6-dimethyl-2-(2-[[3 S,4R]-dimethyl-1-oxophospholidin-1-yl]-2-(4-hydroxy-3-methoxy-5-methylphenyl)ethoxy)pyrimidine;
4,6-dimethyl-2-(2-[P,P-diprop-1-ylphosphinyl]-3-(4-hydroxy-3-methoxy-5-methylphenyl)prop-1-ylamino)pyrimidine;
4,6-dimethyl-2-(2-[P-ethoxy-P-(N-ethylamino)phosphinyl]-2-(4-hydroxy-3-methoxy-5-methylphenyl)cyclohex-1-ylamino)pyrimidine;
4,6-dimethyl-2-(2-[2-oxo-1,3,2-dioxaphospholidin-2-yl]-2-(4-hydroxy-3,5-dimethylphenyl)ethylamino)pyrimidine;
4,6-dimethyl-2-(2-[[4S,5R]-dimethyl-2-oxo-1,3,2-dioxaphospholidin-2-yl]-2-[5-(4-hydoxyphenyl)thien-2-yl]ethylamino)pyrimidine;
4,6-dimethyl-2-(2-[P-ethyl-P-ethoxyphosphinyl 1-oxophosphinan-1-yl]-2-[5-(hydroxymethylbenzimidazol-2-yl]ethylamino)pyrimidine;
3-(2-[5,5-dimethyl-2-oxo-1,2-oxaphosphinan-2-yl]-2-(4-hydroxy-3,5-dimethoxyphenyl)ethylamino)quinoline;
3-(2-[[5R]-methyl-2-oxo-1,2-oxaphospholidin-2-yl]-2-(4-hydroxy-3-methoxy-5-methylphenyl)ethylamino)quinoline;
3-(2-([4R]methyl-2-oxo-1,3,2-oxazaphosphinan-2-yl)-2-(4-hydroxy-3,5-dimethylphenyl)ethylamino)quinoline;
3-(2-([4S]-ethyl-2-oxo-1,3,2-oxazaphospholidin-2-yl)-2-(4-hydroxy-3,5-dimethylphenyl)ethylamino)quinoline;
3-(3-[1-oxophosphinan-1-yl]-3-(4-hydroxy-3,5-dimethylphenyl)prop-1-yl)quinoline;
3-(2-[[3S,4R]-dimethyl-1-oxophospholidin-1-yl]-2-(4-hydroxy-3,5-dimethylphenyl)ethoxy)quinoline;
3-(2-[[4S,5S]-dimethyl-2-oxo-1,3,2-oxazaphospholidin-2-yl]-3-(4-hydroxy-3,5-dimethylphenyl)prop-2-en-1-ylamino)quinoline;
3-(2-[P-ethoxy-P-(N-ethylamino)phosphinyl]-2-(4-hydroxy-3,5-dimethylphenyl)cyclohex-1-ylamino)quinoline;
3-(2-[1N-methyl-2-oxo-1,2-azaphosphinan-2-yl]-2-(indolin-2-on-5-yl)ethylamino)quinoline;
3-(2-[2-oxo-1,3,2-dioxaphospholidin-2-yl]-2-[6-methoxybenzothien-2-yl]ethylamino)quinoline;
3-(2-[[4S,5R]-dimethyl-2-oxo-1,3,2-dioxaphospholidin-2-yl]-2-(4-hydroxy-3-methoxy-5-methylphenyl)ethylamino)quinoline;
3-(2-[4[P-ethyl-P-ethoxyphosphinyl]-2-(5-hydroxy-4-methylimidazol-2-yl)ethylamino)quinoline;
3-(2-[3,5,5-trimethyl-2-oxo-1,3,2-oxazaphosphinan-2-yl]-2-(5-hydroxy-4-methylimidazol-2-yl)ethylamino)quinoline;
3-(2-([4S,5 S]-dimethyl-2-oxo-1,3,2-oxazaphospholidin-2-yl)-2-(5-hydroxy-4-methylimidazol-2-yl)ethylamino)quinoline;
3-(3-[5,5-dimethyl-2-oxo-1,2-oxaphosphinan-2-yl]-3-(5-hydroxy-4-methylimidazol-2-yl)prop-1-yl)quinoline;
3-(2-[[5R]-methyl-2-oxo-1,2-oxaphospholidin-2-yl]-2-(5-hydroxy-4-methylimidazol-2-yl)ethoxy)quinoline;
3-(2-[[3S,4R]-dimethyl-1-oxophospholidin-1-yl]-3-(5-hydroxy-4-methylimidazol-2-yl)prop-2-en-1-ylamino)quinoline;
3-(2-[PP-diprop-1-ylphosphinyl]-2-(5-hydroxy-4-methylimidazol-2-yl)cyclohex-1-ylamino)quinoline;
3-(2-[P-ethoxy-P-(N-ethylamino)phosphinyl]-2-[5-(4-hydoxyphenyl)thien-2-yl]ethyl amino)quino line;
3-(2-[P-ethyl-P-(N-ethylamino)phosphinyl 2-[5-(hydroxymethylbenzimidazol-2-yl]ethylamino)quinoline;
3-(2-[2-oxo-1,2-azaphospholidin-2-yl]-2-(4-hydroxy-3,5-dimethoxyphenyl)ethylamino)quinoline;
3-(2-[2-oxo-1,3,2-dioxaphospholidin-2-yl]-2-(4-hydroxy-3,5-dimethylphenyl)ethylamino)quinoline;
3-(2-[4[S]-methyl-1-thionophospholidin-1-yl]-2-(5-[N-methylamino]thiazol-2-yl)ethylamino)quinoline;
3-(2-[P-(N-ethylamino)-P-propoxyphosphinyl]-2-(5-[N-methylamino]thiazol-2-yl)ethylamino)quinoline;
3-(2-[2-oxo-1,2-oxaphospholidin-2-yl]-2-(5-[N-methylamino]thiazol-2-yl)ethylamino)quinoline;
3-(3-[4[S]-methyl-1-thionophospholidin-1-yl]-3-(5-[N-methylamino]thiazol-2-yl)prop-1-yl)quinoline;
3-(2-[2-oxo-1,2-azaphospholidin-2-yl]-3-(5-[N-methylamino]thiazol-2-yl)prop-1-ylamino)quinoline;
3-(2-[4[S]-methyl-1-thionophospholidin-1-yl]-3-(5-[N-methylamino]thiazol-2-yl)prop-2-en-1-ylamino)quinoline;
3-(2-[[4S,5R]-dimethyl-2-oxo-1,3,2-dioxaphospholidin-2-yl]-2-(indolin-2-on-5-yl)ethylamino)quinoline;
3-(2-[P-ethyl-P-ethoxyphosphinyl]-2-[6-methoxybenzothien-2-yl]ethylamino)quinoline;
3-(2-[[5R]-methyl-2-oxo-1,2-oxaphospholidin-2-yl]-2-(4-hydroxy-3-methoxy-5-methylphenyl)ethylamino)quinoline;
3-(2-[1-oxophosphinan-1-yl]-2-(5-hydroxy-4-methylimidazol-2-yl)ethylamino)quinoline;
3-(2-[3,4-dimethyl-1-thionophospholidin-1-yl]-2-(indolin-2-on-5-yl)ethylamino)quinoline;
3-(2-([4R]-methyl-2-oxo-1,2-azaphosphinan-2-yl)-2-(indolin-2-on-5-yl)ethylamino)quinoline;
3-(2-[[5S]-methyl-2-oxo-1,2-oxaphospholidin-2-yl]-2-(indolin-2-on-5-yl)ethylamino)quinoline;
3-(2-[PP-diprop-1-ylphosphinyl]-2-(indolin-2-on-5-yl)prop-1-yl)quinoline;
3-(2-[2-oxo-1,3,2-dioxaphospholidin-2-yl]-3-(indolin-2-on-5-yl)prop-1-ylamino)quinoline;
3-(2-[5,5-dimethyl-2-oxo-1,3,2-dioxaphosphinan-2-yl]-3-(indolin-2-on-5-yl)prop-2-en-1-ylamino)quinoline;
3-(2-[P-ethyl-P-ethoxyphosphinyl]-2-[5-(4-hydoxyphenyl)thien-2-yl]ethylamino)quinoline;
3-(2-[2-oxo-1,2-oxaphospholidin-2-yl]-2-[5-(hydroxymethylbenzimidazol-2-yl]ethylamino)quinoline;
3-(2-[[5R]-methyl-2-oxo-1,2-oxaphospholidin-2-yl]-2-(4-hydroxy-3,5-dimethoxyphenyl)ethylamino)quinoline;
3-(2-[1-thionphosphinan-1-yl]-2-(4-hydroxy-3,5-dimethoxyphenyl)ethylamino)quinoline;
3-(2-([3S,4S]-dimethyl-2-oxo-1,2-azaphospholidin-2-yl)-2-(4-hydroxy-3,5-dimethoxyphenyl)ethylamino)quinoline;
3-(2-[4,5-dimethyl-2-oxo-1,2-oxaphospholidin-2-yl]-2-(4-hydroxy-3,5-dimethoxyphenyl)ethylamino)quinoline;
3-(2-[P,P-diprop-1-ylphosphinyl]-2-(4-hydroxy-3,5-dimethoxyphenyl)ethoxy)quinoline;
3-(2-[[4S,5S]-dimethyl-2-oxo-1,3,2-oxazaphospholidin-2-yl]-3-(4-hydroxy-3,5-dimethoxyphenyl)prop-2-en-1-ylamino)quinoline;
3-(2-[P-ethyl-P-(N-ethylamino)phosphinyl]-2-(4-hydroxy-3,5-dimethoxyphenyl)cyclohex-1-ylamino)quinoline;
3-(2-[1N-methyl-2-oxo-1,2-azaphosphinan-2-yl]-2-(4-hydroxy-3-methoxy-5-methylphenyl)ethylamino)quinoline;
3-(2-[2-oxo-1,3,2-dioxaphospholidin-2-yl]-2-(indolin-2-on-5-yl)ethylamino)quinoline;
3-(2-[5,5-dimethyl-2-oxo-1,3,2-dioxaphosphinan-2-yl]-2-[6-methoxybenzothien-2-yl]ethylamino)quinoline;
5-methyl-2-(2-[P-ethyl-P-ethoxyphosphinyl]-2-(4-hydroxy-3-methoxy-5-methylphenyl)ethylamino)pyrazine;
5-methyl-2-(2-[2-oxo-1,2-oxaphospholidin-2-yl]-2-(5-hydroxy-4-methylimidazol-2-yl)ethylamino)pyrazine;
5-methyl-2-(2-[1-thionophospholidin-1-yl]-2-[5-(4-hydoxyphenyl)thien-2-yl]ethylamino)pyrazine;
5-methyl-2-(2-[P-ethyl (N-ethylamino)phosphinyl]-2-[5-(4-hydoxyphenyl)thien-2-yl]ethylamino)pyrazine;
5-methyl-2-(2-[2-oxo-1,2-oxaphosphinan-2-yl]-2-[5-(4-hydoxyphenyl)thien-2-yl]ethylamino)pyrazine;
5-methyl-3-(2-[5,5-dimethyl-2-oxo-1,2-oxaphosphinan-2-yl]-3-[5-(4-hydoxyphenyl)thien-2-yl]prop-1-yl)pyrazine;
5-methyl-2-(2-[1-oxophosphinan-1-yl]-3-[5-(4-hydoxyphenyl)thien-2-yl]prop-1-ylamino)pyrazine;
5-methyl-2-(2-[[3 S,4R]-dimethyl-1-oxophospholidin-1-yl]-3-[5-(4-hydoxyphenyl)thien-2-yl]prop-2-en-1-ylamino)pyrazine;
5-methyl-2-(2-[[4S,5S]-dimethyl-2-oxo-1,3,2-oxazaphospholidin-2-yl]-2-[5-(4-hydoxyphenyl)thien-2-yl]ethylamino)pyrazine;
5-methyl-2-(2-[P-ethoxy-P-(N-ethylamino)phosphinyl]-2-[6-methoxybenzothien-2-yl]ethylamino)pyrazine;
5-methyl-2-(2-[2-oxo-1,3,2-dioxaphospholidin-2-yl]-2-(4-hydroxy-3-methoxyphenyl)ethylamino)pyrazine;
5-methyl-2-(2-[5,5-dimethyl-2-oxo-1,3,2-dioxaphosphinan-2-yl]-2-(4-hydroxy-3-methoxy-5-methylphenyl)ethylamino)pyrazine;
5-methyl-2-(2-[P-ethyl-P-ethoxyphosphinyl]-2-(indolin-2-on-5-yl)ethylamino)pyrazine;
5-methyl-2-(2-[2-oxo-1,2-oxaphospholidin-2-yl]-2-[6-methoxybenzothien-2-yl]ethylamino)pyrazine;
5-methyl-2-(2-([4R]-methyl-2-oxo-1,3,2-diazaphosphinan-2-yl)-2-[6-methoxybenzothien-2-yl]ethylamino)pyrazine;
5-methyl-2-(2-([4S]-ethyl-2-oxo-1,3,2-diazaphospholidin-2-yl)-2-[6-methoxybenzothien-2-yl]ethylamino)pyrazine;
5-methyl-3-(2-[5,5-dimethyl-2-oxo-1,2-oxaphosphinan-2-yl]-3-[6-methoxybenzothien-2-yl]prop-1-yl)pyrazine;
5-methyl-2-(2-[[5R]-methyl-2-oxo-1,2-oxaphospholidin-2-yl]-2-[6-methoxybenzothien-2-yl]ethoxy)pyrazine;
5-methyl-2-(2-[4[R]-methyl-1-oxophospholidin-1-yl]-3-[6-methoxybenzothien-2-yl]prop-2-en-1-ylamino)pyrazine;
5-methyl-2-(2-[PP-diprop-1-ylphosphinyl]-2-[6-methoxybenzothien-2-yl]cyclohex-1-ylamino)pyrazine;
5-methyl-2-(2-[P-ethoxy-P-(N-ethylamino)phosphinyl]-2-(4-hydroxy-3-methylphenyl)ethylamino)pyrazine;
5-methyl-2-(2-[P-ethyl-P-(N-ethylamino)phosphinyl]-2-(4-hydroxy-3,5-dimethoxyphenyl)ethylamino)pyrazine;
5-methyl-2-(2-[2-oxo-1,2-azaphospholidin-2-yl]-2-(5-[N-methylamino]thiazol-2-yl)ethylamino)pyrazine;
5-methyl-2-(2-[2-oxo-1,3,2-dioxaphospholidin-2-yl]-2-[5-(4-hydoxyphenyl)thien-2-yl]ethylamino)pyrazine;
5-methyl-2-(2-[1,3,5,5-tetramethyl-2-oxo-1,3,2-diazaphosphinan-2-yl]-2-[5-(hydroxymethylbenzimidazol-2-yl]ethylamino)pyrazine;
5-methyl-2-(2-([4S,5S]-dimethyl-2-oxo1,3,2-diazaphospholidin-2-yl)-2-[5-(hydroxymethylbenzimidazol-2-yl]ethylamino)pyrazine;
5-methyl-3-(2-[[4S,5R]-dimethyl-2-oxo-1,3,2-dioxaphospholidin-2-yl]-3-[5-(hydroxymethylbenzimidazol-2-yl]prop-1-yl)pyrazine;
5-methyl-2-(2-[P-ethyl-P-ethoxyphosphinyl]-2-[5-(hydroxymethylbenzimidazol-2-yl]ethoxy)pyrazine;
5-methyl-2-(2-[5,5-dimethyl-2-oxo-1,2-oxaphosphinan-2-yl]-3-[5-(hydroxymethylbenzimidazol-2-yl]prop-2-en-1-ylamino)pyrazine;
5-methyl-2-(2-[[5R]-methyl-2-oxo-1,2-oxaphospholidin-2-yl]-2-[5-(hydroxymethylbenzimidazol-2-yl]cyclohex-1-ylamino)pyrazine;
5-methyl-2-(2-[3,4-dimethyl-1-thionophospholidin-1-yl]-2-(4-hydroxy-3-methylphenyl)ethylamino)pyrazine;
5-methyl-2-(2-[bis(N-ethylamino)phosphinyl]-2-(4-hydroxy-3-methylphenyl)ethylamino)pyrazine;
5-methyl-2-(2-[4[R]-methyl-1-oxophospholidin-1-yl]-2-(4-hydroxy-3-methylphenyl)ethylamino)pyrazine;
5-methyl-3-(2-[P,P-diprop-1-ylphosphinyl]-3-(4-hydroxy-3-methylphenyl)prop-1-yl)pyrazine;
5-methyl-2-(2-[P-ethoxy-P-(N-ethylamino)phosphinyl]-3-(4-hydroxy-3-methylphenyl)prop-1-ylamino)pyrazine;
5-methyl-2-(2-[P-ethyl-P-(N-ethylamino)phosphinyl]-3-(4-hydroxy-3-methylphenyl)prop-2-en-1-ylamino)pyrazine;
5-methyl-2-(2-[5,5-dimethyl-2-oxo-1,3,2-dioxaphosphinan-2-yl]-2-(4-hydroxy-3-methoxyphenyl)ethylamino)pyrazine;
5-methyl-2-(2-[[4S,5R]-dimethyl-2-oxo-1,3,2-dioxaphospholidin-2-yl]-2-(4-hydroxy-3-methoxy-5-methylphenyl)ethylamino)pyrazine;
5-methyl-2-(2-[2-oxo-1,2-oxaphospholidin-2-yl]-2-(indolin-2-on-5-yl)ethylamino)pyrazine;
5-methyl-2-(2-[5,5-dimethyl-2-oxo-1,2-oxaphosphinan-2-yl]-2-[5-(hydroxymethylbenzimidazol-2-yl]ethylamino)pyrazine;
5-methyl-2-(2-[1-thionophosphinan-1-yl]-2-(4-hydroxy-3-methoxyphenyl)ethylamino)pyrazine;
5-methyl-2-(2-[1-oxophosphinan-1-yl]-2-(4-hydroxy-3-methoxyphenyl)ethylamino)pyrazine;
5-methyl-2-(2-[PP-diprop-1-ylphosphinyl]-2-(4-hydroxy-3-methoxyphenyl)ethylamino)pyrazine;
5-methyl-3-(2-[[4S,5 S]-dimethyl-2-oxo-1,3,2-oxazaphospholidin-2-yl]-3-(4-hydroxy-3-methoxyphenyl)prop-1-yl)pyrazine;
5-methyl-2-(2-[P-ethyl-P-(N-ethylamino)phosphinyl]-3-(4-hydroxy-3-methoxyphenyl)prop-1-ylamino)pyrazine;
5-methyl-2-(2-[1N-methyl-2-oxo-1,2-azaphosphinan-2-yl]-3-(4-hydroxy-3-methoxyphenyl)prop-2-en-1-ylamino)pyrazine;
5-methyl-2-(2-[1N,3N-dimethyl-2-oxo-1,3,2-diazaphosphinan-2-yl]-2-(4-hydroxy-3,5-dimethoxyphenyl)ethylamino)pyrazine;
5-methyl-2-(2-[1-oxophosphinan-1-yl]-2-(4-hydroxy-3,5-dimethylphenyl)ethylamino)pyrazine;
5-methyl-2-(2-[1N,3N-dimethyl-2-oxo-1,3,2-diazaphosphinan-2-yl]-2-[5-(4-hydoxyphenyl)thien-2-yl]ethylamino)pyrazine;
5-methyl-2-(2-[1-oxophosphinan-1-yl]-2-[5-(hydroxymethylbenzimidazol-2-yl]ethylamino)pyrazine;
2,6-dimethyl-3-(2-[2-oxo-1,3,2-dioxaphospholidin-2-yl]-2-(4-hydroxy-3-methoxy-5-methylphenyl)ethylamino)pyridine;
2,6-dimethyl-3-(2-[5,5-dimethyl-2-oxo-1,3,2-dioxaphosphinan-2-yl]-2-(4-hydroxy-3-methoxy-5-methylphenyl)ethylamino)pyridine;
2,6-dimethyl-3-(2-([4S,5S]-dimethyl-2-oxo-1,3,2-oxazaphospholidin-2-yl)-2-(4-hydroxy-3-methoxy-5-methylphenyl)ethylamino)pyridine;
2,6-dimethyl-3-(2-[3N,5,5-trimethyl-2-oxo-1,3,2-oxazaphosphinan-2-yl]-2-(4-hydroxy-3-methoxy-5-methylphenyl)ethylamino)pyridine;
2,6-dimethyl-3-(2-[5,5-dimethyl-2-oxo-1,3,2-diazaphosphinan-2-yl]-2-(4-hydroxy-3-methoxy-5-methylphenyl)ethylamino)pyridine;
2,6-dimethyl-3-(2-[bis-P-(N,N-dimethylamino)phosphinyl]-2-(4-hydroxy-3-methoxy-5-methylphenyl)ethylamino)pyridine;
2,6-dimethyl-3-(2-[bis-P-(N-ethylamino)phosphinyl]-2-(4-hydroxy-3-methoxy-5-methylphenyl)ethylamino)pyridine;
2,6-dimethyl-3-(2-[P-ethoxy-P-(N-ethylamino)phosphinyl]-2-(4-hydroxy-3-methoxy-5-methylphenyl)ethylamino)pyridine;
3,5-dimethyl-2-(2-[2-oxo-1,3,2-dioxaphospholidin-2-yl]-2-(4-hydroxy-3-methoxy-5-methylphenyl)ethylamino)pyrazine;
3,5-dimethyl-2-(2-[5,5-dimethyl-2-oxo-1,3,2-dioxaphosphinan-2-yl]-2-(4-hydroxy-3-methoxy-5-methylphenyl)ethylamino)pyrazine;
3,5-dimethyl-2-(2-([4S,5S]-dimethyl-2-oxo-1,3,2-oxazaphospholidin-2-yl)-2-(4-hydroxy-3-methoxy-5-methylphenyl)ethylamino)pyrazine;
3,5-dimethyl-2-(2-[3N,5,5-trimethyl-2-oxo-1,3,2-oxazaphosphinan-2-yl]-2-(4-hydroxy-3-methoxy-5-methylphenyl)ethylamino)pyrazine;
3,5-dimethyl-2-(2-[5,5-dimethyl-2-oxo-1,3,2-diazaphosphinan-2-yl]-2-(4-hydroxy-3-methoxy-5-methylphenyl)ethylamino)pyrazine;
3,5-dimethyl-2-(2-[bis-P-(N,N-dimethylamino)phosphinyl]-2-(4-hydroxy-3-methoxy-5-methylphenyl)ethylamino)pyrazine;
3,5-dimethyl-2-(2-[bis-P-(N-ethylamino)phosphinyl]-2-(4-hydroxy-3-methoxy-5-methylphenyl)ethylamino)pyrazine;
3,5-dimethyl-2-(2-[P-ethoxy-P-(N-ethylamino)phosphinyl]-2-(4-hydroxy-3-methoxy-5-methylphenyl)ethylamino)pyrazine;
2,4-dimethyl-5-(2-[2-oxo-1,3,2-dioxaphospholidin-2-yl]-2-(4-hydroxy-3-methoxy-5-methylphenyl)ethylamino)pyrimidine;
2,4-dimethyl-5-(2-[5,5-dimethyl-2-oxo-1,3,2-dioxaphosphinan-2-yl]-2-(4-hydroxy-3-methoxy-5-methylphenyl)ethylamino)pyrimidine;
2,4-dimethyl-5-(2-([4S,5S]-dimethyl-2-oxo-1,3,2-oxazaphospholidin-2-yl)-2-(4-hydroxy-3-methoxy-5-methylphenyl)ethylamino)pyrimidine;
2,4-dimethyl-5-(2-[3N,5,5-trimethyl-2-oxo-1,3,2-oxazaphosphinan-2-yl]-2-(4-hydroxy-3-methoxy-5-methylphenyl)ethylamino)pyrimidine;
2,4-dimethyl-5-(2-[5,5-dimethyl-2-oxo-1,3,2-diazaphosphinan-2-yl]-2-(4-hydroxy-3-methoxy-5-methylphenyl)ethylamino)pyrimidine;
2,4-dimethyl-5-(2-[bis-P-(N,N-dimethylamino)phosphinyl]-2-(4-hydroxy-3-methoxy-5-methylphenyl)ethylamino)pyrimidine;
2,4-dimethyl-5-(2-[bis-P-(N-ethylamino)phosphinyl]-2-(4-hydroxy-3-methoxy-5-methylphenyl)ethylamino)pyrimidine;
2,4-dimethyl-5-(2-[P-ethoxy-P-(N-ethylamino)phosphinyl]-2-(4-hydroxy-3-methoxy-5-methylphenyl)ethylamino)pyrimidine;
2-methyl-5-(2-[2-oxo-1,3,2-dioxaphospholidin-2-yl]-2-(4-hydroxy-3-methoxy-5-methylphenyl)ethylamino)thiazole;
2-methyl-5-(2-[5,5-dimethyl-2-oxo-1,3,2-dioxaphosphinan-2-yl]-2-(4-hydroxy-3-methoxy-5-methylphenyl)ethylamino)thiazole;
2-methyl-5-(2-([4S,5 S]-dimethyl-2-oxo-1,3,2-oxazaphospholidin-2-yl)-2-(4-hydroxy-3-methoxy-5-methylphenyl)ethylamino)thiazole;
2-methyl-5-(2-[3N,5,5-trimethyl-2-oxo-1,3,2-oxazaphosphinan-2-yl]-2-(4-hydroxy-3-methoxy-5-methylphenyl)ethylamino)thiazole;
2-methyl-5-(2-[5,5-dimethyl-2-oxo-1,3,2-diazaphosphinan-2-yl]-2-(4-hydroxy-3-methoxy-5-methylphenyl)ethylamino)thiazole;
2-methyl-5-(2-[bis-P-(N,N-dimethylamino)phosphinyl]-2-(4-hydroxy-3-methoxy-5-methylphenyl)ethylamino)thiazole;
2-methyl-5-(2-[bis-P-(N-ethylamino)phosphinyl]-2-(4-hydroxy-3-methoxy-5-methylphenyl)ethylamino)thiazole; and 2-methyl-5-(2-[P-ethoxy-P-(N-ethylamino)phosphinyl]-2-(4-hydroxy-3-methoxy-5-methylphenyl)ethylamino)thiazole.

In a preferred embodiment of this aspect, the pharmaceutical composition comprises a compound of Formula (I) selected from:
2,6-dimethyl-3-(2-[2-oxo-1,3,2-dioxaphospholidin-2-yl]-2-(4-hydroxy-3-methoxy-5-methylphenyl)ethylamino)pyridine;
2,6-dimethyl-3-(2-[5,5-dimethyl-2-oxo-1,3,2-dioxaphosphinan-2-yl]-2-(4-hydroxy-3-methoxy-5-methylphenyl)ethylamino)pyridine;
2,6-dimethyl-3-(2-([4S,5 S]-dimethyl-2-oxo-1,3,2-oxazaphospholidin-2-yl)-2-(4-hydroxy-3-methoxy-5-methylphenyl)ethylamino)pyridine;
2,6-dimethyl-3-(2-[3N,5,5-trimethyl-2-oxo-1,3,2-oxazaphosphinan-2-yl]-2-(4-hydroxy-3-methoxy-5-methylphenyl)ethylamino)pyridine;
2,6-dimethyl-3-(2-[5,5-dimethyl-2-oxo-1,3,2-diazaphosphinan-2-yl]-2-(4-hydroxy-3-methoxy-5-methylphenyl)ethylamino)pyridine;
2,6-dimethyl-3-(2-[bis-P-(N,N-dimethylamino)phosphinyl]-2-(4-hydroxy-3-methoxy-5-methylphenyl)ethylamino)pyridine;
2,6-dimethyl-3-(2-[bis-P-(N-ethylamino)phosphinyl]-2-(4-hydroxy-3-methoxy-5-methylphenyl)ethylamino)pyridine;
2,6-dimethyl-3-(2-[P-ethoxy-P-(N-ethylamino)phosphinyl]-2-(4-hydroxy-3-methoxy-5-methylphenyl)ethylamino)pyridine;
3,5-dimethyl-2-(2-[2-oxo-1,3,2-dioxaphospholidin-2-yl]-2-(4-hydroxy-3-methoxy-5-methylphenyl)ethylamino)pyrazine;
3,5-dimethyl-2-(2-[5,5-dimethyl-2-oxo-1,3,2-dioxaphosphinan-2-yl]-2-(4-hydroxy-3-methoxy-5-methylphenyl)ethylamino)pyrazine;
3,5-dimethyl-2-(2-([4S,5 S]-dimethyl-2-oxo-1,3,2-oxazaphospholidin-2-yl)-2-(4-hydroxy-3-methoxy-5-methylphenyl)ethylamino)pyrazine;
3,5-dimethyl-2-(2-[3N,5,5-trimethyl-2-oxo-1,3,2-oxazaphosphinan-2-yl]-2-(4-hydroxy-3-methoxy-5-methylphenyl)ethylamino)pyrazine;
3,5-dimethyl-2-(2-[5,5-dimethyl-2-oxo-1,3,2-diazaphosphinan-2-yl]-2-(4-hydroxy-3-methoxy-5-methylphenyl)ethylamino)pyrazine;
3,5-dimethyl-2-(2-[bis-P-(N,N-dimethylamino)phosphinyl]-2-(4-hydroxy-3-methoxy-5-methylphenyl)ethylamino)pyrazine;
3,5-dimethyl-2-(2-[bis-P-(N-ethylamino)phosphinyl]-2-(4-hydroxy-3-methoxy-5-methylphenyl)ethylamino)pyrazine;
3,5-dimethyl-2-(2-[P-ethoxy-P-(N-ethylamino)phosphinyl]-2-(4-hydroxy-3-methoxy-5-methylphenyl)ethylamino)pyrazine;
2,4-dimethyl-5-(2-[2-oxo-1,3,2-dioxaphospholidin-2-yl]-2-(4-hydroxy-3-methoxy-5-methylphenyl)ethylamino)pyrimidine;
2,4-dimethyl-5-(2-[5,5-dimethyl-2-oxo-1,3,2-dioxaphosphinan-2-yl]-2-(4-hydroxy-3-methoxy-5-methylphenyl)ethylamino)pyrimidine;
2,4-dimethyl-5-(2-([4S,5S]-dimethyl-2-oxo-1,3,2-oxazaphospholidin-2-yl)-2-(4-hydroxy-3-methoxy-5-methylphenyl)ethylamino)pyrimidine;
2,4-dimethyl-5-(2-[3N,5,5-trimethyl-2-oxo-1,3,2-oxazaphosphinan-2-yl]-2-(4-hydroxy-3-methoxy-5-methylphenyl)ethylamino)pyrimidine;
2,4-dimethyl-5-(2-[5,5-dimethyl-2-oxo-1,3,2-diazaphosphinan-2-yl]-2-(4-hydroxy-3-methoxy-5-methylphenyl)ethylamino)pyrimidine;
2,4-dimethyl-5-(2-[bis-P-(N,N-dimethylamino)phosphinyl]-2-(4-hydroxy-3-methoxy-5-methylphenyl)ethylamino)pyrimidine;
2,4-dimethyl-5-(2-[bis-P-(N-ethylamino)phosphinyl]-2-(4-hydroxy-3-methoxy-5-methylphenyl)ethylamino)pyrimidine;
2,4-dimethyl-5-(2-[P-ethoxy-P-(N-ethylamino)phosphinyl]-2-(4-hydroxy-3-methoxy-5-methylphenyl)ethylamino)pyrimidine;
2-methyl-5-(2-[2-oxo-1,3,2-dioxaphospholidin-2-yl]-2-(4-hydroxy-3-methoxy-5-methylphenyl)ethylamino)thiazole;
2-methyl-5-(2-[5,5-dimethyl-2-oxo-1,3,2-dioxaphosphinan-2-yl]-2-(4-hydroxy-3-methoxy-5-methylphenyl)ethylamino)thiazole;
2-methyl-5-(2-([4S,5S]-dimethyl-2-oxo-1,3,2-oxazaphospholidin-2-yl)-2-(4-hydroxy-3-methoxy-5-methylphenyl)ethylamino)thiazole;
2-methyl-5-(2-[3N,5,5-trimethyl-2-oxo-1,3,2-oxazaphosphinan-2-yl]-2-(4-hydroxy-3-methoxy-5-methylphenyl)ethylamino)thiazole;
2-methyl-5-(2-[5,5-dimethyl-2-oxo-1,3,2-diazaphosphinan-2-yl]-2-(4-hydroxy-3-methoxy-5-methylphenyl)ethylamino)thiazole;
2-methyl-5-(2-[bis-P-(N,N-dimethylamino)phosphinyl]-2-(4-hydroxy-3-methoxy-5-methylphenyl)ethylamino)thiazole;
2-methyl-5-(2-[bis-P-(N-ethylamino)phosphinyl]-2-(4-hydroxy-3-methoxy-5-methylphenyl)ethylamino)thiazole; and 2-methyl-5-(2-[P-ethoxy-P-(N-ethylamino)phosphinyl]-2-(4-hydroxy-3-methoxy-5-methylphenyl)ethylamino)thiazole.

In another aspect, the invention provides methods for regulating levels of apo(a), Lp(a), hypercholesteremia, overall cholesterol homeostasis, apoB, and/or LDL cholesterol in serum, comprising administering to a subject a compound of the invention, or a pharmaceutically acceptable salt thereof, in an amount effective for regulation. This method of regulation can help prevent, treat, or delay the onset or progression of disease states associated with abnormal serum levels of Lp(a), apo(a), apoB, and/or LDL.

In an embodiment of this aspect, the invention relates to methods of lowering serum apo (a) and Lp(a) levels in a subject comprising administering to a subject in need of such treatment an effective amount of a compound of Formula (I), or pharmaceutically acceptable salt thereof.

In another aspect, the invention relates to methods of treating disease states related to high LDL levels, high ApoB levels, hypercholesteremia, defective overall cholesterol homeostasis, and/or elevated apo (a) and Lp(a) levels in a mammal comprising administering to a mammal in need of such treatment a therapeutically effective amount of a compound of Formula (I), or pharmaceutically acceptable salt thereof.

In another embodiment of this aspect, the method can optionally comprise in combination with the compound of Formula (I), or pharmaceutically acceptable salts thereof, an effective amount of a compound known to regulate HDL, Lp(a), and/or LDL levels in blood. Such known compounds include the non-limiting classes of compounds such as statins, for example, lovastatin, simvastatin, pravastatin, atorvastatin, cerivastatin, and niacin; fibrates, for example, clofibrate, bezafibrate, and gemfibrozil; bile acid sequestrants, for example, cholestryramine; and cholesterol uptake inhibitors, for example, phytosteroids, and ezitimibe.

In another embodiment of this aspect, the method can optionally comprise in combination with the compound of Formula (I), or pharmaceutically acceptable salts thereof, an effective amount of a compound known to lower triglyceride levels in blood. Such known compounds include the non-limiting example of fibrates, for example, clofibrate, bezafibrate, and gemfibrozil.

In another embodiment of this aspect, the method can optionally comprise in combination with the compound of Formula (I), an effective amount of a compound known to lower glucose levels in blood. Such known compounds include the non-limiting classes glitazones, such as rosiglitazone and pioglitazone; sulfonylureas; insulin; and metformin.

In another aspect, the methods of treatment comprising administering a compound of Formula (I), or pharmaceutically acceptable salts thereof, can treat an existing condition or metabolic disease, such as hypercholesteremia, atherosclerosis, syndrome X, and thrombosis.

In another aspect, the methods of treatment can prevent the development or progression of a condition or metabolic disease, such as hypercholesteremia, atherosclerosis, syndrome X, and thrombosis.

In one embodiment the method is used in the treatment or prevention of hypercholesteremia.

In one embodiment the method is used in the treatment or prevention of atherosclerosis.

In one embodiment the method is used in the treatment or prevention of Syndrome X.

In one embodiment the method is used in the treatment or prevention of thrombosis.

In certain embodiments the invention relates to a method for the treatment or prevention of atherosclerosis in a mammal comprising coadministering to a mammal in need of such treatment a therapeutically effective amount of a compound of Formula (I),

or pharmaceutically acceptable salt thereof, in combination with a compound known to reduce LDL cholesterol such as, for example, a statin, or with a compound known to increase HDL cholesterol such as, for example, niacin or a fibrate.

In certain embodiments the invention relates to a method for the treatment or prevention of thrombosis in a mammal comprising administering to a mammal in need of such treatment an effective amount of a compound of Formula (I), or pharmaceutically acceptable salt thereof, in combination with an anti-coagulant such as, for example, Aspirin or Clopidogrel.

Without being limited by the following possible mechanisms of action, the compounds of Formula (I) may be effective in the treatment or prevention of thrombosis by functioning to lower serum apo (a) and Lp(a) levels in a subject. Similarly, the compounds of the invention may be effective in the treatment or prevention of atherosclerosis by functioning to improve overall cholesterol homeostasis, and reducing the levels of ApoB, LDL, apo (a) and Lp(a) in a subject.

In one embodiment of the methods of the invention, the subject is a mammal. In a more preferred embodiment, the mammal is a human.

The methods of the invention employ therapeutically effective amounts: for oral, parenteral, sublingual, intranasal, intrathecal, depo, implants, topical, and rectal administration from about 0.1 mg/day to about 5,000 mg/day. The therapeutically effective amounts will vary according to various parameters including, for example, the particular therapeutic use and physical characteristics of the subject/patient, and are well within the knowledge of those skilled in the art.

In a preferred aspect, the therapeutically effective amounts for oral, parenteral, and depot administration is from about 50 mg/day to about 500 mg/day.

The invention also includes the use of a compound of Formula (I), or a pharmaceutically acceptable salt thereof for the manufacture of a medicament for use in treating a subject who has, or in preventing a subject from developing, a metabolic disease, condition, or disorder, such as, for example, abnormal cholesterol homeostasis, hypercholesteremia, atherosclerosis, hyperglycemia, syndrome X, and thrombosis and who is in need of such treatment.

In one aspect, this use of a compound of formula (I) can be employed where the disease is hypercholesteremia.

In another aspect, this use of a compound of formula (I) can be employed where the disease is atherosclerosis.

In another aspect, this use of a compound of formula (I) can be employed where the disease is syndrome X.

In another aspect, this use of a compound of formula (I) can be employed where the disease is thrombosis.

The invention also includes a container kit including a plurality of containers, each container including one or more unit dose of a compound of formula (I), or a pharmaceutically acceptable salt thereof.

In an embodiment, this container kit includes each container adapted for oral delivery and includes a tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

The invention also includes an agent kit including a compound of formula (I), or a pharmaceutically acceptable salt thereof; and one or more therapeutic agents selected from the group consisting of statins, fibrates, bile acid sequestrants, cholesterol uptake inhibitors, glitazones, sulfonylureas, insulin, and metformin.

The compounds of formula (I) can form salts when reacted with appropriate acids or bases. Pharmaceutically acceptable salts are generally preferred over the corresponding compounds of formula (I) since they frequently produce compounds that are usually more water soluble, stable and/or more crystalline. Pharmaceutically acceptable salts are any salt which retains the activity of the parent compound and does not impart any deleterious or undesirable effect on the subject to whom it is administered and in the context in which it is administered. Pharmaceutically acceptable salts include acid addition salts of both inorganic and organic acids. Preferred pharmaceutically acceptable salts include salts such as those described by Berge, Bighley, and Monkhouse, J. Pharm. Sci., 1977, 66, 1-19. Such salts may be formed from inorganic and organic acids. Representative examples thereof include maleic, fumaric, benzoic, ascorbic, pamoic, succinic, bismethylenesalicylic, methanesulfonic, ethanedisulfonic, acetic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids. For other acceptable salts, see Int. J. Pharm., 33, 201-217 (1986).

Methods of the Invention

The compounds of the invention, pharmaceutical compositions comprising said compounds, and pharmaceutically acceptable salts thereof, are useful for treating mammals suffering from a disease or condition characterized by at least one pathological form of abnormal total cholesterol levels, and/or LDL levels, and/or Lp(a) levels in blood, and are useful for helping to prevent or delay the onset of such a condition. The compounds and compositions of the invention are particularly useful for treating, preventing, or slowing the progression of conditions and/or metabolic disorders, including, for example, hypercholesteremia, atherosclerosis, syndrome X, and thrombosis. When treating or preventing these diseases and conditions, the compounds of the invention can either be used individually or in combination, as is best for the subject.

With regard to these diseases, the term “treating” means that compounds of the invention can be used in subjects, preferably human subjects/patients, with existing disease. The compounds of the invention will not necessarily cure the subject who has the disease but will delay or slow the progression or prevent further progression of the disease thereby giving the individual a more useful life span.

The term “preventing” means that that if the compounds of the invention are administered to those who do not now have the disease but who would normally develop the disease or be at increased risk for the disease, they will not develop the disease. In addition, “preventing” also includes delaying the development of the disease in an individual who will ultimately develop the disease or would be at risk for the disease due to age, familial history, genetic or chromosomal abnormalities, and/or due to the presence of one or more biological markers for the disease. By delaying the onset of the disease, compounds of the invention can prevent the individual from getting the disease during the period in which the individual would normally have gotten the disease or reduce the rate of development of the disease or some of its effects but for the administration of compounds of the invention up to the time the individual ultimately gets the disease. Preventing also includes administration of the compounds of the invention to those individuals thought to have predisposition for the disease.

In a preferred aspect, the compounds of the invention are useful for slowing the progression of disease symptoms.

In another preferred aspect, the compounds of the invention are useful for preventing the further progression of disease symptoms.

In treating or preventing the above diseases, the compounds of the invention are administered in a therapeutically effective amount. The therapeutically effective amount will vary depending on the particular compound used and the route of administration, as is known to those skilled in the art.

In treating a subject displaying any of the diagnosed above conditions a physician may administer a compound of the invention immediately and continue administration indefinitely, as needed.

Dosage Forms and Amounts

The compounds of the invention can be administered orally, parenterally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the subject treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the subject 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

As noted above, depending on whether asymmetric carbon atoms are present, the compounds of the invention can be present as mixtures of isomers, especially as racemates, or in the form of pure isomers, especially optical antipodes.

Salts of compounds are preferably the pharmaceutically acceptable or non-toxic salts of compounds of formula I. For isolation and purification purposes it is also possible to use pharmaceutically unacceptable salts.

Synthesis of Compounds

The compounds useful in the methods of the invention can be synthesized by a wide variety of routes, as exemplified by the procedures set forth in Schemes 1-15 below. Those of skill will appreciate that minor modifications may be made to the specific procedures in that article to arrive at compounds useful in the invention. Further, a wide variety of processes known to those of skill can be useful in the synthesis of the compounds of the invention.

The distinguishing feature of these compounds is a phosphorus-containing moiety joined by a phosphorus-carbon bond to a saturated or partially saturated linker between two aromatic moieties. The linker is most usually 3-5 atoms long, and may contain one heteroatom, chosen from the group N, O or S. If the phosphorus is introduced early in the synthetic scheme, its ability to stabilize negative charge, undergo sigmatropic rearrangements or add across carbonyls can be used to help assemble the skeleton of the molecule.

The phosphorus-containing reagents required for these syntheses can be made in a variety of methods usually starting, conceptually at least, from PCl₃ or POCl₃. Simple phosphite esters and the corresponding phosphinamides and phosphinamidates, and all of the corresponding phosphorus (III) monochlorides are obtained by treatment of PCl₃ sequentially with the appropriate stoichiometries of alcohols or amines, or for cyclic derivatives, diols, aminoalcohols [Sum, V.; Kee, T. P., J. Chem. Soc., Perkin 1, 2701, (1993).], or diamines (Denmark, S. E.; Kim, J. H., Can. J. Chem., 78, 673, (2000). Such a species with one remaining P—Cl bond can be reacted with carbanions or enamines, to directly introduce the desired P—C bond, or with an alcohol, where the resultant P—O bond can be rearranged by a 1,2- or 2,3-sigmatropic shift [Christov, V. C., Phosphorus, Sulfur and Silicon and the Related Elements, 132, 73, (1998). For Ru catalysis see: Ji, H. L. et al., Organometallics, 11, 1618 (1992)]to the desired P—C bond. Alternatively, reduction of the P—Cl bond with a metal, or a metal halogen exchange reaction can produce a strongly nucleophilic P(III) centred anion, which can react with a wide variety of electrophilic carbon atoms to form the desired P—C bond.

Displacement of chloride from POCl₃ or PCl₃ or suitable alkyl/alkoxy/amino substituted derivatives with one or two equivalents of an organometallic compound such as a Grignard reagent or an organolithium species will give mono and di-alkyl phosphine species, which can in the former case be converted to precursors of phosphinates or phosphinamides by treatment with one equivalent of an alcohol or amine, and which are in the latter case precursors for phosphine oxides. Phosphates, [Eymery, F. et al., Tetrahedron, 55, 13109, (2000)], phosphites, phosphonates and phosphinites [Laughlin, R. G., J. Org. Chem., 27, 1005, (1962), Hall, R. G. and Riebli, P., Eur. Pat. App. EP 501702, (1992)]and their amine analogues will also react with organometallic agents to form phosphorus derivatives containing one or more new phosphorus-carbon bonds.

Use of α,ω-dimetallic species leads to precursors for cyclic phosphine oxides [Polniaszek, R. P and Foster, A. L., J. Org. Chem., 56, 3137, (1991)]. Cyclic phosphinate and phosphinamide [eg (1,2-(ox/az)(phospholidines/phosphinanes)]precursors can made via a wide variety of techniques [For 6-membered rings see Maerkl, G. and Kreitmeier, P., Phosphorus-Carbon Heterocyclic Chemistry, Pergamon Press (2001) pp 535-630. Le Floch, P., Phosphorus-Carbon Heterocyclic Chemistry, Pergamon Press (2001) pp 485-533.]. Deprotonation of dimethyl methyl phosphonate, followed by alkylation of the anion with an epoxide, will give a 1,2-oxaphospholidine by spontaneous cyclization of the resultant alkoxide [Collard, J. N. and Benezra, C., Tetrahedron Lett., 3725, (1982)]. The corresponding oxaphosphinane could be made by transmetalation of the anion, Michael addition to propenal, and a mild reduction of the carbonyl to an alcohol, which will again spontaneously cyclize. Reductive amination of the 4-phosphonobutanal would lead to cyclization to the corresponding 1,2-azaphosphinane. Heating trimethyl phosphite with 1,3-dibromoalkanes induces a double Arbuzov reaction, producing a 1,2-oxaphospholane [Bergeson, K., Acta Chem. Scand. 24, 1122, (1970).]. Alternatively, radical addition of a suitable monoalkyl phosphine, across an allyl alcohol, followed by mild oxidation of the remaining P—H bond with NBS or bromine and a base should lead to spontaneous cyclization to the 1,2-oxaphospholine, as such a process has been demonstrated with allylamines to make 1,2-azaphospholines [Oehme, H. and Thamm, R., J. Praktische Chem., 315, 526, (1973). Use of homoallyl alcohols or amines extends the preparation to the corresponding 1,2-(ox/az)aphosphinanes. Reversing the steps, reaction of a 3- or 4-haloalcohol with a chloromethoxy phosphine leads to P—Cl displacement, and cleavage of the methoxy group (see below) unmasks an H-phosphonate, which will cyclize onto the terminal halide in the presence of base. The same displacement on a dichlorophosphine derivative allows cyclization of the phosphorus alkyl dihalide with lithium [Kobayashi, S. et al., Bull. Chem. Soc. Japan, 58, 2153, (1985)]. 1,2,3-Dioxaphosphinanes can also be made from simple diesters by treatment with an appropriate 1,3-diol [Sulsky, R. B. PCT WO 99/021564 (1999)].

Hydrolysis or aminolysis of a P(V) ester, followed by conversion of the phosphonic/phosphenic/hemi ester/amide or acid to an acid chloride produces a P(V)halide [Lorga, B. et al., Comptes Rendu Acad. Sci Serie IIc, 3, 821 (2000).], which can be used to introduce the desired phosphorus entity electrophilically in the synthesis.

P(III) species substituted with one or more alkoxy species can be converted to tetracoordinated phosphine oxide species by cleavage of the C—O alkoxy bond. It this is done as a hydrolysis or aminolysis it leads to P-hydridophosphinyl species, which can readily be deprotonated to give P-centred phosphinyl anions, which can be used to introduce the phosphorus fragment via nucleophilic substitution, addition across carbonyls, or Michael additions. Alternatively this can be done by an initial alkylation of phosphorus, followed by nucleophilic O-dealkylation of the resulting oxyphosphonium species to give phosphonates, as exemplified in the Arbuzov [Henning, H. G. and Hilgetag, G., Zeitschriftfuer Chemie, 7, 169, (1976), Arbuzov, B. A., Pure and Applied Chemistry, 9, 307, (1964)]and Perkow reactions [Borowitz, I. J. and Crouch, R., Phosphorus and the Related Group V Elements, 2, 209, (1973)].

The most important way to introduce phosphorus into the compounds of the present invention is by Arbuzov reactions. Benzylic halides, mesylates etc., (or their heteroaryl equivalents) undergo the Arbuzov reaction readily with methoxy or ethoxy P(III) species to form benzylic phosphonates etc. as exemplified in Schemes 3, 4 & 7.

Another very important process involves the additions of phosphite (H-phosphonate or H-phosphinate) derivatives, [Simoni et al., Tetrahedron Lett., 39, 7615, (1998)]or silylphosphite derivatives [Afarinkiaet al., Tetrahedron, 46, 7175, (1990)] across carbonyl double bonds, the so-called Abramov reaction [Abramov, V. S.; S. M. Kirov, Doklady Akademii Nauk SSSR, 73, 487, (1950)]. Variations described in the literature include additions across aldehydes, ketones, acyl chlorides, [Pohjala et al., U.S. Pat. No. 5,393,748 (1995)], acylphosphonates, [Lecouvey et al., Heteroatom Chemistry, 11, 556, (2000)], amides, [Failla et al., Heteroatom Chemistry, 11, 493, (2000)], imines, [Wolfsberger, W., Chemiker-Zeitung, 109, 317, (1985)]and silylphosphites across the carbonyl of α,β-unsaturated aldehydes and ketones [Evans, D. A. et al., Tetrahedron Lett., 2495 (1977)]. Phosphite species, most of which are known to add across carbonyls, include the H-phosphonate derivatives diesters, cyclic diesters, diamides, [Evans, D. A. et al., Tetrahedron Lett., 2495 (1977)]cyclic diamides, [Pudovik et al., Izvestiya Akademii Nauk SSSR, Seriya Khimi., 1183, (1980)], and chiral cyclic phosphinamidoates, [Sum, V.; Kee, T. P., J. Chem. Soc., Perkin 1, 2701, (1993)], as well as thiophosphites [Pudovik, A. N.; Zametaeva, G. A., Bull. Acad. Sci. U.S.S.R., Classe sci. chim., 825, (1952)], H-phosphinites [Yamagishi, T. et al., Tetrahedron Lett., 42, 5033, (2001)]phosphinites, [Yamagishi et al., Tetrahedron, 59, 767, (2003)], silylaminophosphines, [Heilson, R. H.; Goebel, D. W. J. Chem. Soc., Chemical Commun., 769, (1979)], phosphine oxides and phosphine sulfides. [Goerlich, J. R.; Schmutzler, R., Phosphorus, Sulfur and Silicon and the Related Elements, 101, 213, (1995)]. Similar transformations can be carried out by using trialkyl phosphite esters and POCl₃, [Olive, G.; Jacques, A., Phosphorus, Sulfur and Silicon and the Related Elements, 178, 33, (2003)], or phosphorus (III) halides and acetic acid, [Conant, J. B. et al., J. Amer. Chem Soc., 45, 165, (1923)]. The α-hydroxy(bis)phosphonate derivatives obtained in several of these processes may be converted to other useful synthetic intermediates, such as the 1,1-bisphosphonates, 1-mesyloxyphosphonates and 1-benzoyloxyphosphonates, by methods described in the literature.

Dialkylphosphites exist largely as the H-phosphonate tautomers, and these can readily be turned into the corresponding phosphorus-based anions by bases [Troev. K., Reviews on Heteroatom Chemistry, 13, 99, (1995)]. These anions are involved in many of the additions across carbonyls described above, but they can also be converted directly to C-phosphonates by alkylation [Kem, K. M. et al., J. Org. Chem., 46, 5188, (1981), Ilea, G. et al., Revista de Chimie, 52, 101, (2001), Kers, A. et al., Tetrahedron, 53, 12691, (1997)]. The phosphorus anion allows for P-alkylation even for thionophosphonate derivatives [Swierczek, K. et al., Tetrahedron, 59, 595, (2003), Lin, Q. et al., Qingdao Huagong Xueyuan Xuebao, 19, 403, (1998)]. Similarly, di-H-phosphinates can be P-alkylated once to form H-phosphinates, which can be P-alkylated again to form phosphinate esters [Gallagher, M. J.; Ranasinghe, M. G., Phosphorus, Sulfur and Silicon and the Related Elements, 115, 255, (1996)]. Ammonium hypophosphite can be dialkylated with alkyl halides in the presence of hexamethyldisilazane to produce trimethylsilyl phosphinate esters, both acyclic and cyclic [Kurdyumova, N. R. et al., Zh. Obshch. Khim., 64, 419, (1994)]. The alkylation can also be through Michael addition to many common α,β-unsaturated systems [Castelot-Deliencourt, G. et al., Eur. J. Org. Chem., 3031, (2001), Simoni, D. et al., Tetrahedron Lett., 39, 7615, (1998) Mastryukova, T. A. et al., Izvestiya Akad. Nauk SSSR, Seriya Khim., 1164, (1972)]The selective cleavage of many phosphite esters by amines at the α-carbon atom, allows for simple phosphites to be unmasked as H-phosphonates, and subsequently turned into phosphoryl anions or phosphoryl halides [Troev, K., Rev. Heteroatom Chemistry, 11, 89, (1994), Saady, M. et al., Tetrahedron Lett., 36, 4785, (1995)].

The ability of phosphinyl species to stabilize a negative charge at the neighbouring atom means that H-atoms on an α-carbon are readily base abstracted, to give carbanions which can be reacted with a wide variety of electrophiles to extend the chain, as illustrated in Schemes 2, 3, 4, 6 and 7. This same charge-stabilizing ability allows phosphonates to activate double bonds towards Michael addition, such as is illustrated in Schemes 11 & 12. Vinyl phosphonates and diphosphonates are straightforwardly prepared by a number of routes [Minami, T. and Motoyoshiya, J., Synthesis, 333, (1992)]. Ring-opening of phosphonyl-substituted cyclopropanes [Hirao, T. et al., Bull. Chem. Soc. Japan, 58, 3104, (1985), Dolhaine, H and Haergele, G., Phosphorus, Sulfur and Silicon and the Related Elements, 4, 123, (1978)]will employ the same carbanion-stabilizing ability and is used in Scheme 5. The use of diphosphonyl species to react with carbonyl groups to make vinyl phosphonates is also a precedented reaction, and is illustrated in Schemes 2 & 12. The sigmatropic allyl phosphenate-phosphonate rearrangement has been used to make allyl phosphonates, and these in turn can be deprotonated and reacted at the less hindered terminus with electrophiles as illustrated in Scheme 6. Another way that α-phosphinyl carbanions can be produced is by reduction of an electron-accepting group in that position, and this is illustrated in Scheme 8 by SmI₂ reduction of a benzoate [J. Org. Chem., 65, 475, (2000), Chinese Chem. Lett. 2, 673, (1991)]. In this case the Lewis acidic nature of the Sm counterion allows the hydroxyphosphonate to be isolated, and a double bond formed without loss of phosphorus. In Scheme 10, mesylate is eliminated α to phosphorus to produce a π-allyl Pd cation, which is then nucleophilically attacked [Ohler, E. and Kanzler, S. Liebigs Annalen/Recueil, 1437 (1997), and the same is done with allyl carbonates [De La Cruz, M. A. et al., Phosphorus, Sulfur, Silicon and the Related Elements, 144-6, 181, (1999)] in Scheme 15.

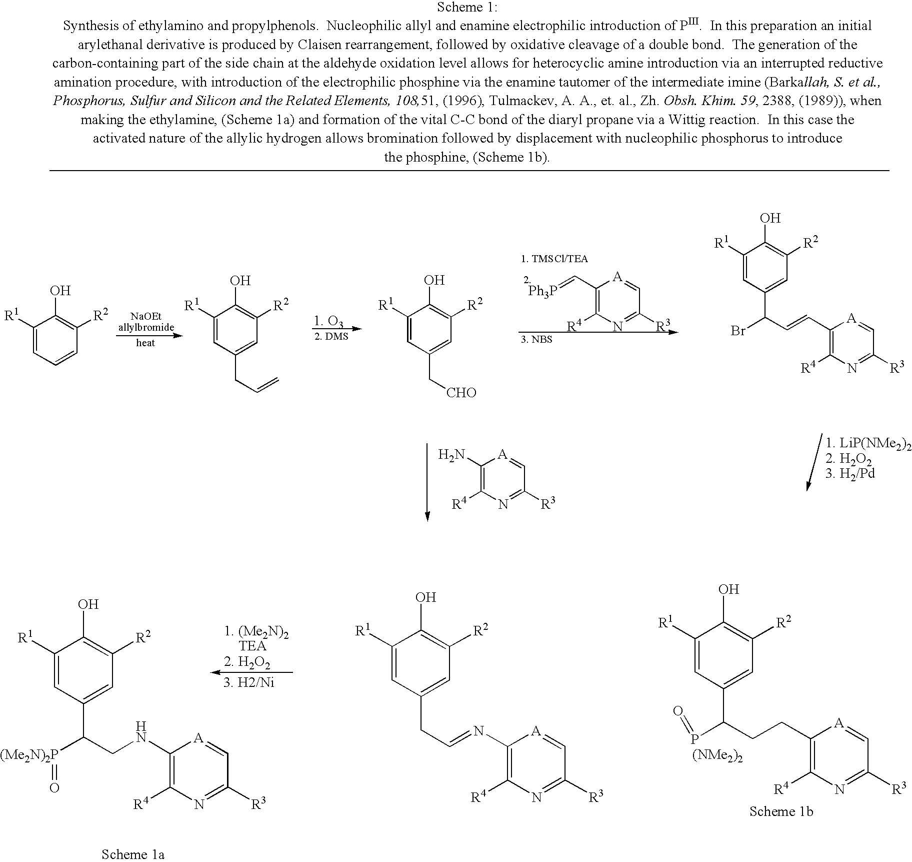

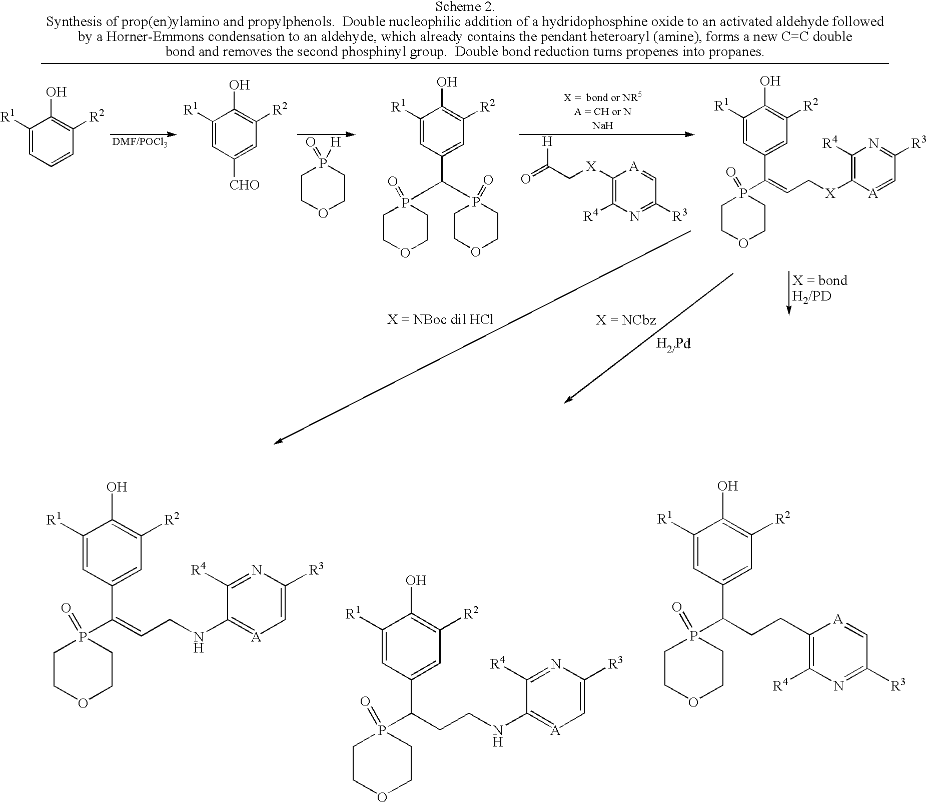

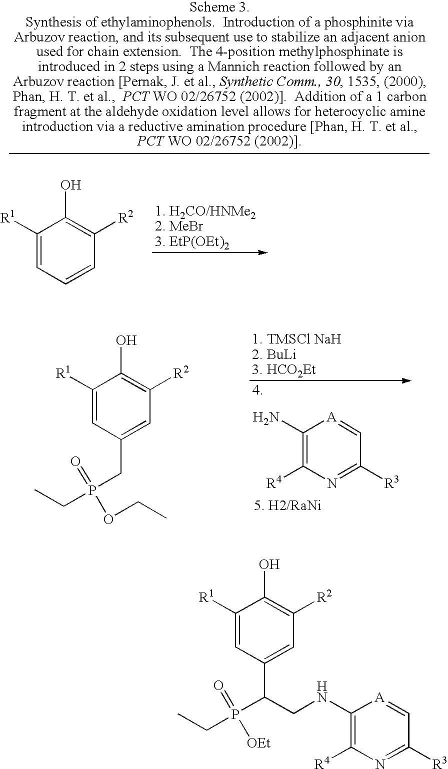

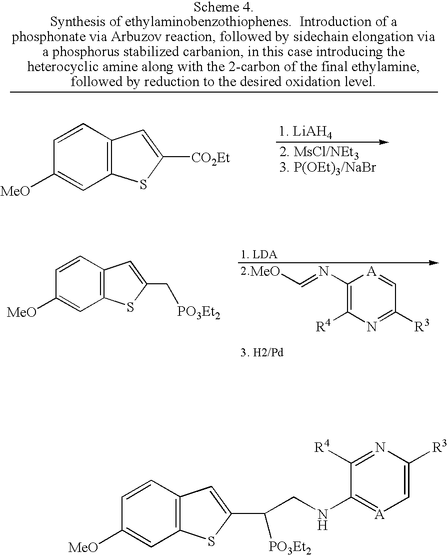

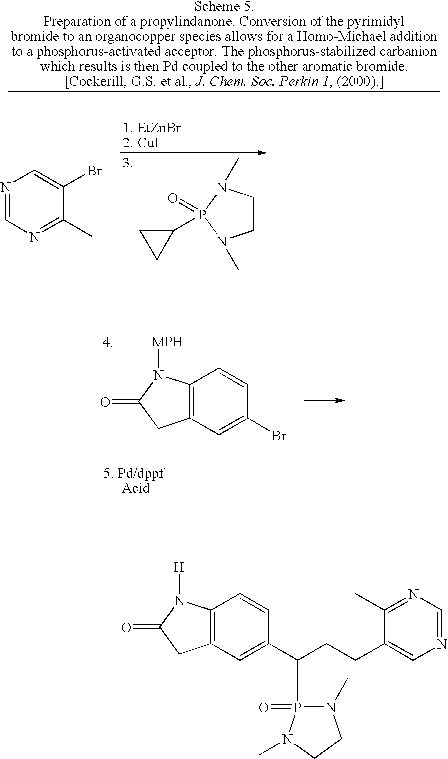

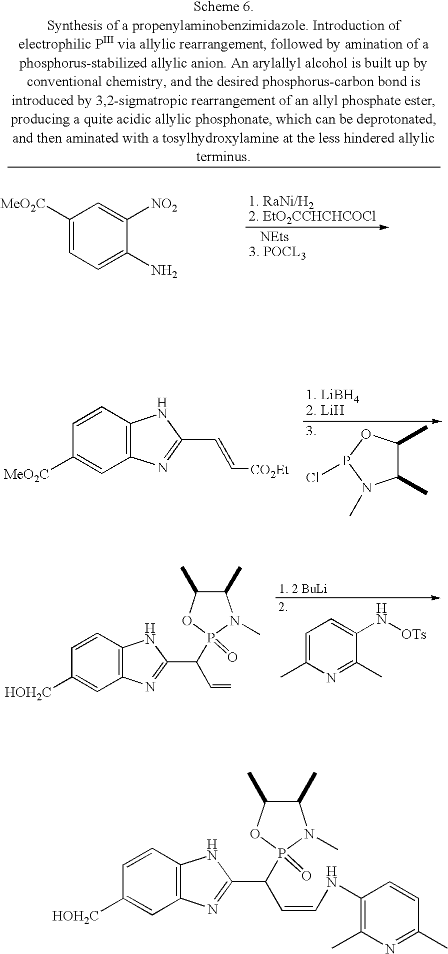

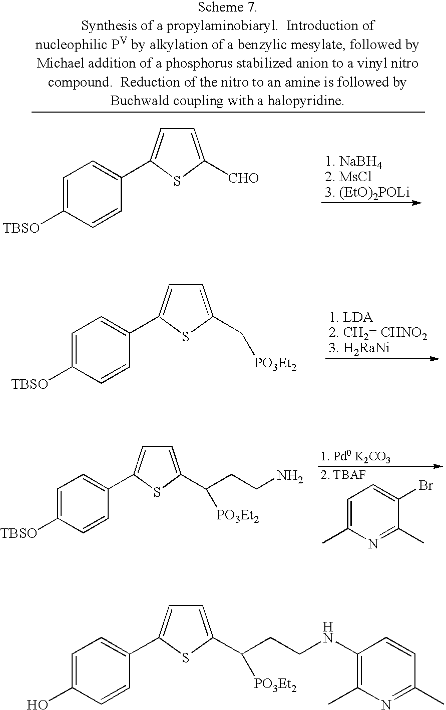

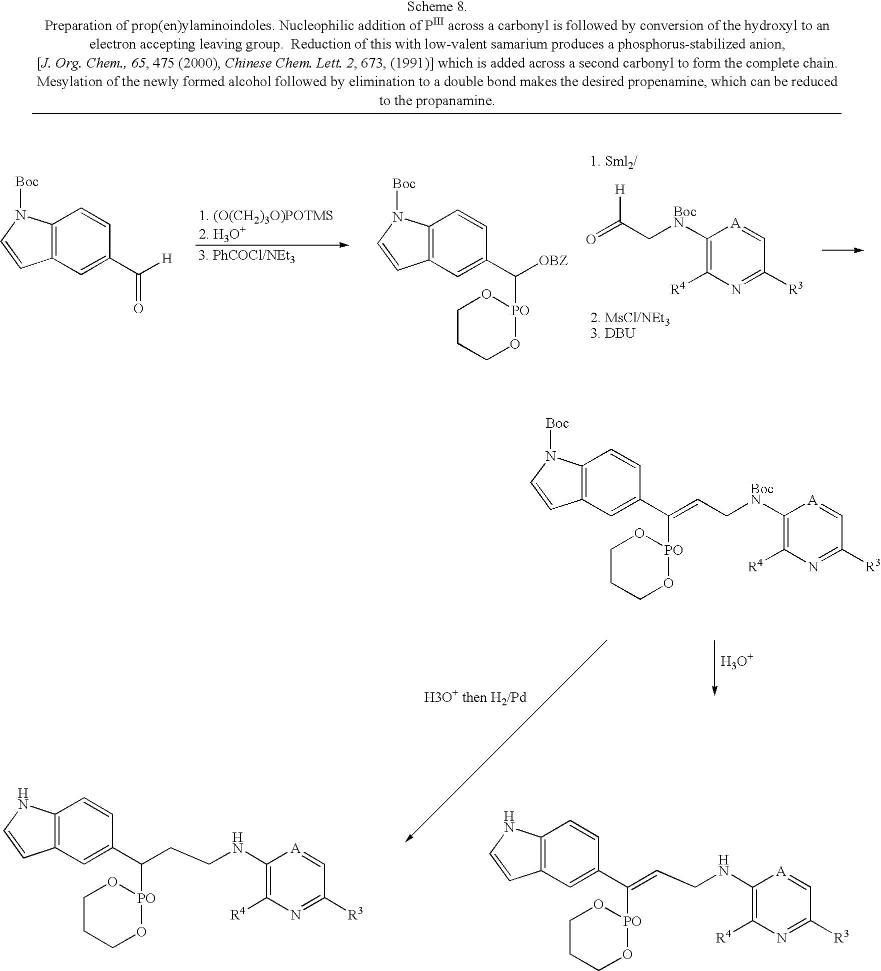

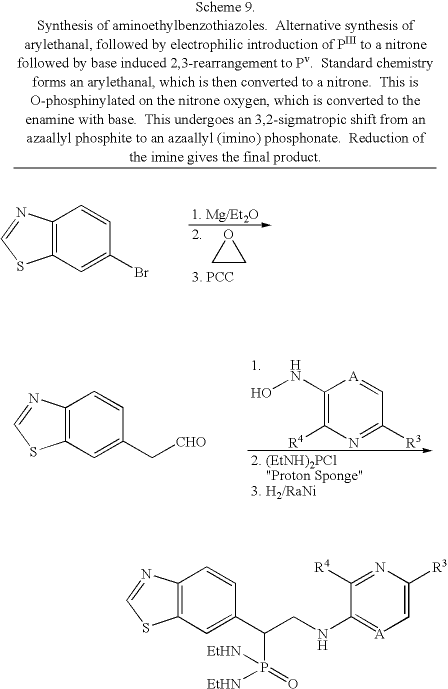

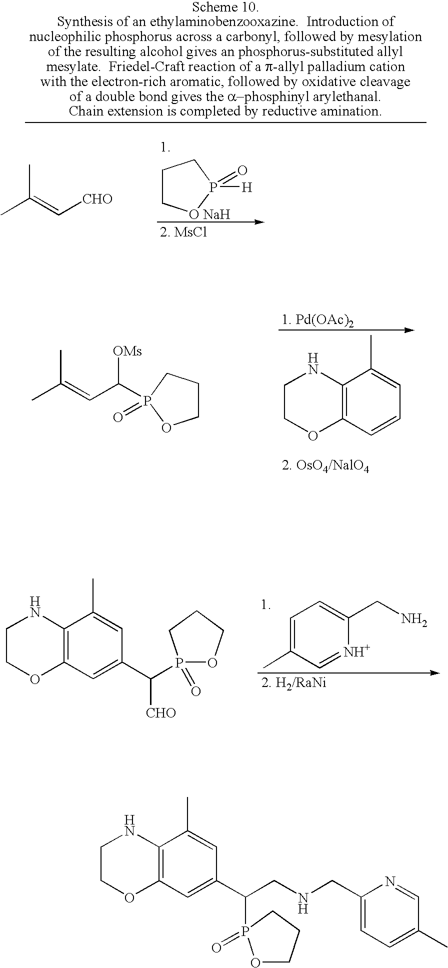

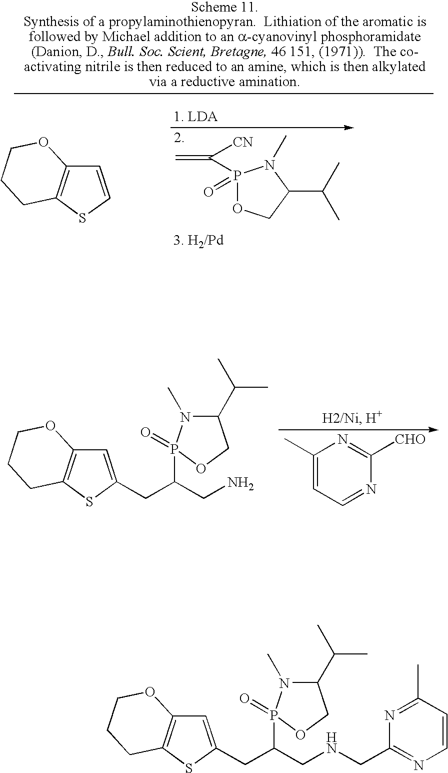

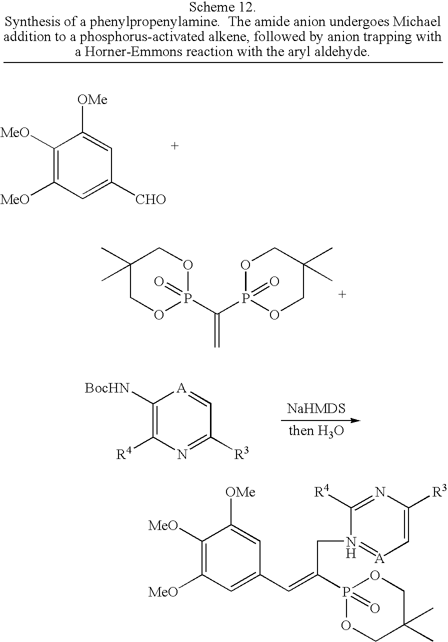

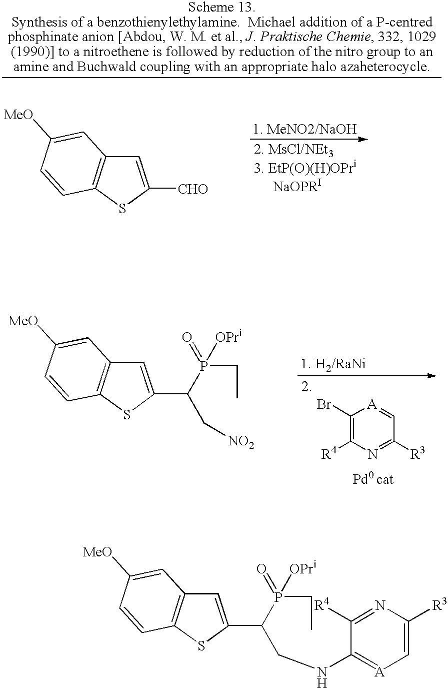

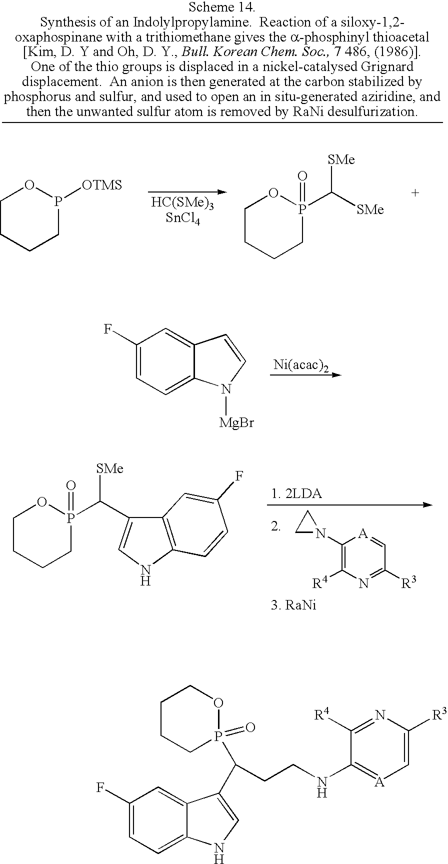

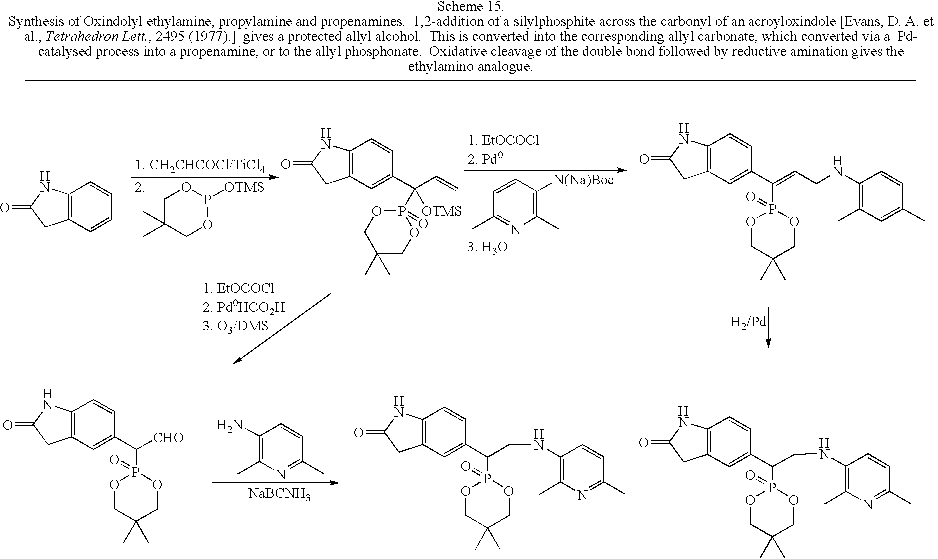

It is understood that the foregoing description is provided to illustrate particular embodiments of the instant invention, and should not be viewed as narrowing the scope or spirit of the invention as defined by the appended claims.

1. A compound of Formula (I): 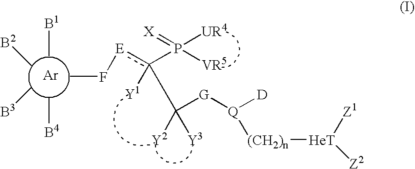
wherein
Ar is a 5-10 membered mono- or bicyclic aromatic ring containing 0-4 heteroatoms selected from the group consisting of N, O and S, with the proviso that any given ring contains a maximum of one S or one O atom;
B¹-B⁴ are independently, R¹, OR¹, NR¹R², S(O)ₙR₁, SO₂NR¹R², OCOR¹, NR¹COR², NR¹CO₂R², OCO₂R¹, NR¹CONR¹R², COR¹, CONR¹R², CH₂OR₁, CH₂NR¹R², R_{f}, OR_{f}, S(O)ₘR_{f}, CN, NO₂, F, Cl, Br, I, monocyclic aromatic,
wherein the monocyclic aromatic is phenyl, pyridyl, pyrimidyl, pyrazinyl, pyridazinyl, thienyl, furanyl, pyrrolyl, imidazyl, pyrazolyl, oxazolyl, isoxoazolyl, thiazolyl, isothiazolyl, 1,3,4-oxadiazolyl, 1,2,4-oxadiazolyl, 1,3,4-thiadiazolyl, 1,2,3-triazolyl, 1,3,4-triazolyl, 1,2,4-triazinyl, 1,3,5-triazinyl, each optionally substituted with Z¹;
or any two of B¹-B⁴ on contiguous atoms of the aromatic ring may be taken together to form a 5-8 membered partially saturated ring (which is fused to the Ar ring) that optionally includes up to two heteroatom groups selected from NR³, O, and S(O)ₘ, and where said partially saturated ring can be optionally substituted by up to 2 groups selected independently from R³, OR³, NR³R^{3′}, F, Cl, Br, S(O)ₙR³, CN, NO₂, and ═O;
R¹ and R² are independently H, C₁-C₈ lower alkyl, C₂-C₈ lower alkenyl, C₂-C₈ lower alkynyl, C₃-C₈ cycloalkyl, C₃-C₈ cycloalkenyl, or NR⁷R⁸, wherein R⁷ and R⁸ are independently H, C₁-C₈ lower alkyl, C₂-C₈ lower alkenyl, C₂-C₈ lower alkynyl, C₃-C₈ cycloalkyl, C₃-C₈ cycloalkenyl, or can optionally be taken together with the N to which they are bound to form a 3-8 membered azacyclic ring that optionally includes an additional heteroatom group selected from NR³, O, or S(O)ₘ, and wherein R¹ and R² are each independently optionally substituted by up to 4 groups selected from the group consisting of R³, OR³, NR³R³, F, Cl, Br, S(O)ₙR³, CN, NO₂, and =0;
R_{f} is C₁-C₄ straight or branched lower perfluoroalkyl;
D is nothing, R³, COR³, or COR_{f};
R³ and R³′ are independently H, C₁-C₄ lower alkyl, C₂-C₄ lower alkenyl, or C₂-C₄ alkynyl, or NR⁹R¹⁰, where R⁹ and R¹⁰ are independently H, C₁-C₄ lower alkyl, C₂-C₄ lower alkenyl, or C₂-C₄ alkynyl, or can optionally be taken together with the N to which they are bound to form a 3-8 membered azacyclic ring that optionally includes an additional heteroatom group selected from the NR³, O or S(O)ₘ, wherein R³ and R³ are optionally substituted with up to 2 groups selected from R⁶, OR⁶, SR⁶, CF₃, OCF₃, NR⁶R⁶′, =O, hydroxy, F, Cl, Br, I, CN, and NO₂;
R⁶ and R⁶′ are independently H, or C₁-C₃ lower alkyl;
E′ is a carbon;
E is nothing, or is a methine doubly bonded to E′, with the proviso that when E is a methine doubly bonded to E′, Y¹ is nothing;
F is a bond, (CH₂)ₙ, or —CH═CH—;
G is a bond or CR³R^{3′}
Het is a 5-10 membered mono- or bicyclic heteroaromatic ring containing at least one nitrogen atom;
m is 0, 1, or 2;
n is 0, 1, 2, or 3;
Q is N, CR³R³, O, S(O)ₘ, or a bond, with the proviso that when D is R³, COR³ or COR_{f}, Q is N;
R⁴ and R⁵ are each independently H, C₁-C₆ lower alkyl, C₃-C₆ lower alkenyl, C₃-C₆ lower alkynyl, C₃-C₆ lower cycloalkyl, C₄-C₆ lower cycloalkenyl, or R⁴ and R⁵ can be taken together with U, V, and the phosphorus to which they are attached, to form a 5 to 8-membered ring containing 2-7 carbon atoms, and up to 2 heteroatom groups selected from O, S(O)ₘ or NR³, wherein the 5 to 8-membered ring is optionally substituted with up to 3 substituents selected from R³, OR³, SR³, NR³R^{3′}, CF₃, OCF₃, phenyl, substituted phenyl, benzyl, substituted benzyl, ═O, and C₁-C₄ alkylidene;
U and V are each independently a bond, O, or NR³;
X is O or S;
Y¹, Y², and Y³ are each independently, H, C₁-C₆ lower alkyl, C₂-C₆ lower alkenyl, C₂-C₆ lower alkynyl, C₃-C₆ lower cycloalkyl, C₄-C₆ lower cycloalkenyl, or optionally any two of Y¹, Y², and Y³ taken together with the carbons to which they are bound can form a 3-8 membered saturated ring that optionally includes one heteroatom group selected from NR³, O, and S(O)ₘ, or Y¹ and Y² taken together can form a double bond between E′ and the carbon to which Y and Y are attached, or if E is a methine doubly bonded to E′, Y¹ is nothing; and
Z¹ and Z² are independently, H, C₁-C₆ lower alkyl, C₂-C₆ lower alkenyl, C₂-C₆ lower alkynyl, C₃-C₆ lower cycloalkyl, C₄-C₆ lower cycloalkenyl, OR³, NR³R³, COR³, CONR³R³, S(O)ₙR³, R_{f}, OR_{f}, S(O)ₘR_{f}, F, Cl, Br, I, CN, or NO₂;
or Z¹ and Z², when vicinally substituted, can be taken together to form a partially saturated ring of 5-7 atoms that is fused to the Het group, and optionally contains 1 or 2 heteroatoms independently selected from O, S(O)ₘ, and NR³;
with a first independent proviso that:
when Ar is phenyl, and
when B¹-B⁴ are each independently selected from R¹, OR¹, CH₂OR³, NR³R³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³contain only single bonds, and
when D contains only single bonds, and
when E is nothing, and
when F is (CH₂)₀₋₂, and
when Q is N, and
when U, V and X are all O, and
when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or with a second independent proviso that
when Ar is phenyl, and
when B¹-B⁴ are independently selected from the group R¹, OR¹, CH₂OR³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
when D is nothing, and
when E is nothing or CH, in which case Y¹ is nothing, and
when F is (CH₂)₀₋₂ or —CH═CH—, and
when Q is a bond, and
when U, V and X are all O, and
when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or a pharmaceutically acceptable salt thereof.

2. The compound according to claim 1, wherein
Ar is phenyl, thienyl, furanyl, pyrrolyl, imidazyl, pyrazolyl, oxazolyl, isoxoazolyl, thiazolyl, isothiazolyl, benzofuranyl, benzothienyl, indolyl, indazolyl, benzimidazyl, benzoxazoyl, benzoisozazolyl, benzothiazoyl, benzoisothiazolyl, naphthyl, quinolinyl, isoquinolinyl pyrrolothienyl, furanothienyl, or thienothienyl.

3. A compound according to claim 1, wherein
Het is pyrrolyl, imidazyl, pyrazolyl, oxazolyl, isoxoazolyl, thiazolyl, isothiazolyl, 1,3,4-oxadiazolyl, 1,2,4-oxadiazolyl, 1,3,4-thiadiazolyl, 1,2,3-triazolyl, 1,3,4-triazolyl, pyridyl, pyrimidyl, pyrazinyl, pyridazinyl, 1,2,4-triazinyl, 1,3,5-triazinyl, pyrrolothienyl, pyrrolooxazolyl, pyrrolothiazolyl, pyrroloimidazolyl, pyrrolopyrazolyl, indolyl, indazolyl, benzimidazyl, benzoxazoyl, benzoisozazolyl, benzothiazoyl, benzoisothiazolyl, pyrrolopyridyl, thienopyridyl, furanopyridyl, pyrrolopyrimidyl, thienopyrimidyl, furanopyrimidyl, oxazolopyridyl, oxazolopyrimidyl, thiazolopyridyl, thiazolopyrimidyl, imidazolopyridyl, purinyl, quinolinyl, isoquinolinyl, quinazolinyl, quinoxalinyl, cinnolinyl, phthalazinyl, naphthyridinyl, or pyridopyrimidyl.

4. A compound according to Formula (I), 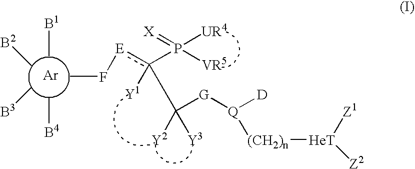
wherein
Ar is phenyl, thienyl, furanyl, pyrrolyl, imidazyl, oxazolyl, thiazolyl, benzofuranyl, benzothienyl, indolyl, benzimidazyl, benzoxazoyl, benzothiazoyl, or naphthyl;
B¹-B⁴ are independently R¹, OR₁, CH₂OR³, OCOR¹, NR¹COR², NR¹R³, F, Cl, Br or I, or any two of B¹-B⁴ on contiguous aromatic positions may be taken together to form a 5-7 membered partially saturated ring, which optionally includes up to two heteroatoms selected from NR³, O, and S(O)ₘ, and said ring may be optionally substituted by up to 2 groups selected independently from R³, OR³, NR¹R^{3′}, F, Cl, Br, S(O)n)R³, CN, NO₂, and ═O;
R¹ and R² are independently H, C₁-C₈ lower alkyl, C₂-C₈ lower alkenyl, C₂-C₈₂₀ lower alkynyl, C₃-C₈ cycloalkyl, C₃-C₈ cycloalkenyl, or NR⁷R⁸, wherein R⁷ and R⁸ are independently H, C₁-C₈ lower alkyl, C₂-C₈ lower alkenyl, C₂-C₈ lower alkynyl, C₃-C₈ cycloalkyl, C₃-C₈ cycloalkenyl, or can optionally be taken together with the N to which they are bound to form a 3-8 membered azacyclic ring that optionally includes an additional heteroatom group selected from NR³, O, or S(O)ₘ, and wherein R¹ and R² are each independently optionally substituted by up to 4 groups selected from the group consisting of R³, OR³, NR³R^{3′}, F, Cl, Br, S(O)ₙR³, CN, NO₂, and ═O;
D is R³;
R³ and R³′ are independently H, C₁-C₄ lower alkyl, C₂-C₄ lower alkenyl, or C₂-C₄ alkynyl, or NR⁹R¹⁰, where R⁹ and R¹⁰ are independently H, C₁-C₄ lower alkyl, C₂-C₄ lower alkenyl, or C₂-C₄ alkynyl, or can be taken together with the N to which they are bound to form a 3-8 membered azacyclic ring that optionally includes an additional heteroatom group selected from the NR³, O or S(O)ₘ, wherein R³ and R³ are optionally substituted with up to 2 groups selected from R⁶, OR⁶, SR⁶, CF₃, OCF₃, NR⁶R^{6′}, ═O, hydroxy, F, Cl, Br, I, CN, and NO₂;
R⁶ and R⁶′ are independently H, or C₁-C₃ lower alkyl;
E′ is a carbon;
E is nothing, or is a methine doubly bonded to E′, with the proviso that when E is a methine doubly bonded to E′, Y¹ is nothing;
F is a bond;
G is a bond or CR³₂;
Het is pyridyl, pyrazinyl, pyrimidyl, pyridazinyl, imidazyl, oxazolyl, thiazolyl, or 1,2,4-triazinyl;
m is 0, 1 or 2;
n is 0 or 1;
Q is N, CR³R^{3′}, or O;
R⁴ and R⁵ are each independently C₁-C₆ lower alkyl, C₃-C₆ lower alkenyl, C₃-C₆ lower alkynyl, C₃-C₆ lower cycloalkyl, C₄-C₆ lower cycloalkenyl, or R⁴ and R⁵ can be taken together with U, V, and the phosphorus to which they are attached, to form a 5 to 8-membered ring containing 2-7 carbon atoms, and up to 2 heteroatoms selected from O, S(O)ₘ or NR³, wherein the 5 to 8-membered ring is optionally substituted with up to 3 substituents selected from R³, OR³, SR³, NR³R³′;
U and V are each independently O or NR³;
X is 0;
Y¹, Y², and Y³ are each independently H, C₁-C₆ lower alkyl, C₂-C₆ lower alkenyl, C₂-C₆ lower alkynyl, C₃-C₆ lower cycloalkyl, C₄-C₆ lower cycloalkenyl, or Y¹ and Y² taken together can form a double bond betweem E′ and the carbon to which Y² and Y³ are attached, or if E is a methine doubly bonded to E′, Y¹ is nothing; and
Z¹ and Z² are independently lone pair, H, C₁-C₆ lower alkyl, C₂-C₆ lower alkenyl, C₂-C₆ lower alkynyl, C₃-C₆ lower cycloalkyl, C₄-C₆ lower cycloalkenyl, OR³, NR³R^{3′}, COR³, CONR³R^{3′}, S(O)R³, R_{f}, OR_{f}, S(O)ₘR_{f}, F, Cl, Br, I, CN NO₂;
or Z¹ and Z, when vicinally substituted, can be taken together to form a partially saturated ring of 5-7 atoms that is fused to the Het group, and optionally contains 1 or 2 heteroatoms independently selected from O, S(O)ₘ, and NR³;
with a first independent proviso that:
when Ar is phenyl, and
when B¹-B⁴ are each independently selected from R¹, OR¹, CH₂OR³, NR³R³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³contain only single bonds, and
when D contains only single bonds, and
when E is nothing, and
when F is (CH₂)₀₋₂, and
when Q is N, and
when U, V and X are all O, and
when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or with a second independent proviso that
when Ar is phenyl, and
when B¹-B⁴ are independently selected from the group R¹, OR¹, CH₂OR³, CN, F, Cl, Br, I and NO₂, or two of B¹-B⁴ on contiguous atoms of the Ar ring are taken together form a 5 or 6-membered cycloalkyl or alkylidenedioxy ring, wherein R¹ and R³contain only single bonds, and
when D is nothing, and
when E is nothing or CH, in which case Y¹ is nothing, and
when F is (CH₂)₀₋₂ or —CH═CH—, and
when Q is a bond, and
when U, V and X are all O, and
when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or a pharmaceutically acceptable salt thereof.

5. A compound according to Formula (I), 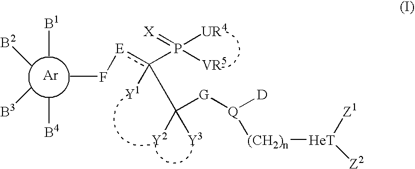
wherein
Ar is phenyl, thienyl, imidazyl, oxazolyl or thiazolyl;
B¹-B⁴ are independently R¹, OR₁, CH₂OR³, OCOR¹, NR¹COR², NRR 3, F, Cl, Br or I,
D is R³;
R¹ and R² are independently H, C₁-C₈ lower alkyl, C₂-C₈ lower alkenyl, C₂-C₈ lower alkynyl, C₃-C₈ cycloalkyl, C₃-C₈ cycloalkenyl, or NR⁷R⁸, wherein R⁷ and R⁸ are independently H, C₁-C₈ lower alkyl, C₂-C₈ lower alkenyl, C₂-C₈ lower alkynyl, C₃-C₈ cycloalkyl, C₃-C₈ cycloalkenyl, or can optionally be taken together with the N to which they are bound to form a 3-8 membered azacyclic ring that optionally includes an additional heteroatom group selected from NR³, O, or S(O)ₘ, and wherein R¹ and R² are each independently optionally substituted by up to 4 groups selected from the group consisting of R³, OR³, NR³R³, F, Cl, Br, S(O)ₙR³, CN, NO₂, and ═O;
R³ and R³ are independently H, C₁-C₄ lower alkyl, C₂-C₄ lower alkenyl, or C₂-C₄ alkynyl, or NR⁹R¹⁰, where R⁹ and R¹⁰ are independently H, C₁-C₄ lower alkyl, C₂-C₄ lower alkenyl, or C₂-C₄ alkynyl, or can be taken together with the N to which they are bound to form a 3-8 membered azacyclic ring that optionally includes an additional heteroatom group selected from the NR³, O or S(O)ₘ, wherein R³ and R³ are optionally substituted with up to 2 groups selected from R⁶, OR⁶, SR⁶, CF₃, OCF₃, NR⁶R^{6′}, ═O, hydroxy, F, Cl, Br, I, CN, and NO₂;
R⁶ and R⁶ are independently H, or C₁-C₃ lower alkyl;
E′ is a carbon;
E is nothing;
F is a bond;
G is a bond or CR³R³′;
Het is pyridyl, pyrazinyl, pyrimidyl, pyridazinyl, imidazyl, oxazolyl, thiazolyl or 1,2,4-triazinyl
m is 0, 1 or 2;
n is 0;
Q is N, CR³R^{3′}, or O;
R⁴ and R⁵ are each independently C₁-C₆ lower alkyl, C₃-C₆ lower alkenyl, C₃-C₆ lower alkynyl, C₃-C₆ lower cycloalkyl, C₄-C₆ lower cycloalkenyl, or R⁴ and R⁵ can be taken together with U, V, and the phosphorus to which they are attached, to form a 5 to 8-membered ring containing 2-7 carbon atoms, and up to 2 heteroatoms selected from O, S(O)ₘ or NR³, wherein the 5 to 8-membered ring is optionally substituted with up to 3 substituents selected from R³, OR³, SR³, NR³R³;
U and V are each independently O or NR³;
X is O;
Y¹, Y², and Y are each independently H, C₁-C₆ lower alkyl, C₂-C₆ lower alkenyl, C₂-C₆ lower alkynyl, or Y¹ and Y² taken together can form a double bond between E′ and the carbon to which Y² and Y³ are attached; and
Z¹ and Z² are independently lone pair, H, C₁-C₆ lower alkyl, OR³, NR³R³, COR³, CoNR³R^{3′}, S(O)ₘR³, R_{f}, OR_{f}, S(O)ₘR_{f}, F, Cl, Br, I;
with a first independent proviso that:
when Ar is phenyl, and
when B¹-B⁴ are each independently selected from R¹, OR₁, CH₂OR³, NR³R³, CN, F, Cl, Br, I and NO₂, or two of B¹-B⁴ on contiguous atoms of the Ar ring are taken together form a 5- or 6-membered cycloalkyl or alkylidenedioxy ring, wherein R¹ and R³ contain only single bonds, and
when D contains only single bonds, and
when E is nothing, and
when F is (CH₂)₀₋₂, and
when Q is N, and
when U, V and X are all O, and
when yl Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or with a second independent proviso that
when Ar is phenyl, and
when B¹-B⁴ are independently selected from the group R¹, OR¹, CH₂OR³, CN, F, Cl, Br, I and NO₂, or two of B¹-B⁴ on contiguous atoms of the Ar ring are taken together form a 5 or 6-membered cycloalkyl or alkylidenedioxy ring, wherein R¹ and R³ contain only single bonds, and
when D is nothing, and
when E is nothing or CH, in which case Y¹ is nothing, and
when F is (CH₂)₀₋₂ or —CH═CH—, and
when Q is a bond, and
when U, V and X are all O, and
when Y¹-Y³ are H or C₁-C₄ lower alkyl, or Y¹ and Y² taken together are a double bond and Y³ is H or C₁-C₄ lower alkyl,
then one of R⁴ and R⁵ is not H or unsubstituted C₁-C₆ straight or branched chain lower alkyl;
or a pharmaceutically acceptable salt thereof.

6. A compound according to claim 1, wherein the compound is selected from the group consisting of:
2,6-dimethyl-3-(2-[diethoxyphosphinyl]-2-(5-hydroxy-4-methylimidazol-2-yl)ethylamino)pyridine;
2,6-dimethyl-3-(2-[diethoxyphosphinyl]-2-(5-[N-methylamino]thiazol-2-yl)ethylamino)pyridine;
2,6-dimethyl-3-(2-[diethoxyphosphinyl]-2-[5-(4-hydoxyphenyl)thien-2-yl]ethylamino)pyridine;
2,6-dimethyl-3-(2-[diethoxyphosphinyl]-2-[6-methoxybenzothien-2-yl]ethylamino)pyridine;
2,6-dimethyl-3-(2-[diethoxythiophosphinyl]-2-(5-hydroxy-4-methylimidazol-2-yl)ethylamino)pyridine;
2,6-dimethyl-3-(2-[diethoxythiophosphinyl]-2-(5-[N-methylamino]thiazol-2-yl) ethyl amino)pyridine;
2,6-dimethyl-3-(2-[diethoxythiophosphinyl]-2-[5-(4-hydoxyphenyl)thien-2-yl]ethylamino)pyridine;
2,6-dimethyl-3-(2-[diethoxythiophosphinyl]-2-[6-methoxybenzothien-2-yl]ethylamino)pyridine;
2,6-dimethyl-3-(2-[1N,3N-dimethyl-2-oxo-1,3,2-diazaphosphinan-2-yl]-2-(5-hydroxy-4-methylimidazol-2-yl)ethylamino)pyridine;
2,6-dimethyl-3-(2-[1N,3N-dimethyl-2-oxo-1,3,2-diazaphosphinan-2-yl]-2-(5-[N-methylamino]thiazol-2-yl)ethylamino)pyridine;
2,6-dimethyl-3-(2-[1N,3N-dimethyl-2-oxo-1,3,2-diazaphosphinan-2-yl]-2-[5-(4-hydoxyphenyl)thien-2-yl]ethylamino)pyridine;
2,6-dimethyl-3-(2-[1N,3N-dimethyl-2-oxo-1,3,2-diazaphosphinan-2-yl]-2-[6-methoxybenzothien-2-yl]ethylamino)pyridine;
2,6-dimethyl-3-(2-[[4S,5R]-dimethyl-2-oxo-1,3,2-diazaphospholidin-2-yl]-2-(5-hydroxy-4-methylimidazol-2-yl)ethylamino)pyridine;
2,6-dimethyl-3-(2-[[4S,5R]-dimethyl-2-oxo-1,3,2-diazaphospholidin-2-yl]-2-(5-[N-methylamino]thiazol-2-yl)ethylamino)pyridine;
2,6-dimethyl-3-(2-[[4S,5R]-dimethyl-2-oxo-1,3,2-diazaphospholidin-2-yl]-2-[5-(4-hydoxyphenyl)thien-2-yl]ethylamino)pyridine;
2,6-dimethyl-3-(2-[[4S,5R]-dimethyl-2-oxo-1,3,2-diazaphospholidin-2-yl]-2-[6-methoxybenzothien-2-yl]ethylamino)pyridine;
2,6-dimethyl-3-(2-[bis-P-(N-ethylamino)phosphinyl]-2-(5-hydroxy-4-methylimidazol-2-yl)ethylamino)pyridine;
2,6-dimethyl-3-(2-[bis-P-(N-ethylamino)phosphinyl]-2-(5-[N-methylamino]thiazol-2-yl)ethylamino)pyridine;
2,6-dimethyl-3-(2-[bis-P-(N-ethylamino)phosphinyl]-2-[5-(4-hydoxyphenyl)thien-2-yl]ethylamino)pyridine;
2,6-dimethyl-3-(2-[bis-P-(N-ethylamino)phosphinyl]-2-[6-methoxybenzothien-2-yl]ethylamino)pyridine;
2,6-dimethyl-3-(2-[2-oxo-1,3,2-dioxaphospholidin-2-yl]-2-(4-hydroxy-3-methylphenyl)ethylamino)pyridine;
2,6-dimethyl-3-(2-[1-thionoophospholidin-1-yl]-2-(4-hydroxy-3-methylphenyl)ethylamino)pyridine;
2,6-dimethyl-3-(2-[5,5-dimethyl-2-oxo-1,3,2-dioxaphosphinan-2-yl]-2-(4-hydroxy-3-methylphenyl)ethylamino)pyridine;
2,6-dimethyl-3-(3-[[4S,5R]-dimethyl-2-oxo-1,3,2-dioxaphospholidin-2-yl]-3-(4-hydroxy-3-methylphenyl)prop-1-yl)pyridine;
2,6-dimethyl-3-(2-[2-oxo-1,2-oxaphospholidin-2-yl]-3-(4-hydroxy-3-methylphenyl)prop-1-ylamino)pyridine;
2,6-dimethyl-3-(2-[5,5-dimethyl-2-oxo-1,2-oxaphosphinan-2-yl]-3-(4-hydroxy-3-methylphenyl)prop-2-en-1-ylamino)pyridine;
2,6-dimethyl-3-(2-[1-oxophosphinan-1-yl]-2-(4-hydroxy-3-methoxyphenyl)ethylamino)pyridine;
2,6-dimethyl-3-(2-[[3 S,4R]-dimethyl-1-oxophospholidin-1-yl]-2-(4-hydroxy-3-methoxy-5-methylphenyl)ethylamino)pyridine;
2,6-dimethyl-3-(2-[[4S,5S]-dimethyl-2-oxo-1,3,2-oxazaphospholidin-2-yl]-2-(indolin-2-on-5-yl)ethylamino)pyridine;
2,6-dimethyl-3-(2-[P-ethoxy-P-(N-ethylamino)phosphinyl]-2-[6-methoxybenzothien-2-yl]ethylamino)pyridine;
2,6-dimethyl-3-(2-[4[R]-methyl-1-thionophospholidin-1-yl]-2-(4-hydroxy-3-methoxyphenyl)ethylamino)pyridine;
2,6-dimethyl-3-(2-[1N-methyl-2-oxo-1,2-azaphosphinan-2-yl]-2-(4-hydroxy-3-methoxyphenyl)ethylamino)pyridine;
2,6-dimethyl-3-(3-[bis-[P-(N,N-dimethylamino)]phosphinyl]-3-(4-hydroxy-3-methoxyphenyl)prop-1-yl)pyridine;
2,6-dimethyl-3-(2-[4[R]-methyl-1-oxophospholidin-1-yl]-2-(4-hydroxy-3-methoxyphenyl)ethoxy)pyridine;
2,6-dimethyl-3-(2-[5,5-dimethyl-2-oxo-1,3,2-dioxaphosphinan-2-yl]-3-(4-hydroxy-3-methoxyphenyl)prop-2-en-1-ylamino)pyridine;
2,6-dimethyl-3-(2-[[4S,5R]-dimethyl-2-oxo-1,3,2-dioxaphospholidin-2-yl]-2-(4-hydroxy-3-methoxyphenyl)cyclohex-1-ylamino)pyridine;
2,6-dimethyl-3-(2-[2-oxo-1,2-oxaphospholidin-2-yl]-2-(4-hydroxy-3,5-dimethylphenyl)ethylamino)pyridine;
2,6-dimethyl-3-(2-[5,5-dimethyl-2-oxo-1,2-oxaphosphinan-2-yl]-2-(5-[N-methylamino]thiazol-2-yl)ethylamino)pyridine;
2,6-dimethyl-3-(2-[1-oxophosphinan-1-yl]-2-[5-(hydroxymethylbenzimidazol-2-yl]ethylamino)pyridine;
2,6-dimethyl-3-(2-[[3 S,4R]-dimethyl-1-oxophospholidin-1-yl]-2-(4-hydroxy-3-methylphenyl)ethylamino)pyridine;
2,6-dimethyl-3-(2-[4[S]-methyl-1-thionophospholidin-1-yl]-2-(4-hydroxy-3,5-dimethoxyphenyl)ethylamino)pyridine;
2,6-dimethyl-3-(2-[P-ethoxy-P-(N-ethylamino)phosphinyl 2-(4-hydroxy-3,5-dimethoxyphenyl)ethylamino)pyridine;
2,6-dimethyl-3-(3-[[4S,5S]-dimethyl-2-oxo-1,3,2-oxazaphospholidin-2-yl]-3-(4-hydroxy-3,5-dimethoxyphenyl)prop-1-yl)pyridine;
2,6-dimethyl-3-(2-[1N-methyl-2-oxo-1,2-azaphosphinan-2-yl]-2-(4-hydroxy-3,5-dimethoxyphenyl)ethoxy)pyridine;
2,6-dimethyl-3-(2-[4[S]-methyl-1-oxophospholidin-1-yl]-3-(4-hydroxy-3,5-dimethoxyphenyl)prop-2-en-1-ylamino)pyridine;
2,6-dimethyl-3-(2-[4[S]-methyl-1-oxophospholidin-1-yl]-2-(4-hydroxy-3,5-dimethoxyphenyl)cyclohex-1-ylamino)pyridine;
2,6-dimethyl-3-(2-[5,5-dimethyl-2-oxo-1,3,2-dioxaphosphinan-2-yl]-2-(5-hydroxy-4-methylimidazol-2-yl)ethylamino)pyridine;
2,6-dimethyl-3-(2-[[4S,5R]-dimethyl-2-oxo-1,3,2-dioxaphospholidin-2-yl]-2-(indolin-2-on-5-yl)ethylamino)pyridine;
2,6-dimethyl-3-(2-[2-oxo-1,2-oxaphospholidin-2-yl]-2-(4-hydroxy-3-methoxyphenyl)ethylamino)pyridine;
2,6-dimethyl-3-(2-[5,5-dimethyl-2-oxo-1,2-oxaphosphinan-2-yl]-2-(4-hydroxy-3-methoxy-5-methylphenyl)ethylamino)pyridine;
2,6-dimethyl-3-(2-[diethylphosphosphiny]-2-(4-hydroxy-3-methoxy-5-methylphenyl)ethylamino)pyridine;
2,6-dimethyl-3-(2-[[5R]-methyl-2-oxo-1,2-oxaphospholidin-2-yl]-2-(4-hydroxy-3-methoxy-5-methylphenyl)ethylamino)pyridine;
2,6-dimethyl-3-(2-[[3S,4R]-dimethyl-1-oxophospholidin-1-yl]-2-(4-hydroxy-3-methoxy-5-methylphenyl)ethoxy)pyridine;
2,6-dimethyl-3-(2-[P,P-diprop-1-ylphosphinyl]-3-(4-hydroxy-3-methoxy-5-methylphenyl)prop-1-ylamino)pyridine;
2,6-dimethyl-3-(2-[P-ethoxy-P-(N-ethylamino)phosphinyl]-2-(4-hydroxy-3-methoxy-5-methylphenyl)cyclohex-1-ylamino)pyridine;
2,6-dimethyl-3-(2-[P-ethyl-P-(N-ethylamino)phosphinyl]-2-(4-hydroxy-3,5-dimethylphenyl)ethylamino)pyridine;
2,6-dimethyl-3-(2-[bis-[P-(N,N-dimethylamino)]phosphinyl]-2-[5-(4-hydoxyphenyl)thien-2-yl]ethylamino)pyridine;
2,6-dimethyl-3-(2-[3,4-dimethyl-1-oxophospholidin-1-yl]-2-[5-(hydroxymethylbenzimidazol-2-yl]ethylamino)pyridine;
2,6-dimethyl-3-(2-[5,5-dimethyl-2-oxo-1,3,2-dioxaphosphinan-2-yl]-2-(4-hydroxy-3,5-dimethoxyphenyl)ethylamino)pyridine;
2,6-dimethyl-3-(2-[[4S,5R]-dimethyl-2-oxo-1,3,2-dioxaphospholidin-2-yl]-2-(4-hydroxy-3,5-dimethylphenyl)ethylamino)pyridine;
2,6-dimethyl-3-(2-[P-ethyl-P-ethoxyphosphinyl]-2-(4-hydroxy-3,5-dimethylphenyl)ethylamino)pyridine;
2,6-dimethyl-3-(2-[2-oxo-1,2-oxaphospholidin-2-yl]-2-(4-hydroxy-3,5-dimethylphenyl)ethylamino)pyridine;
2,6-dimethyl-3-(2-[[5R]-methyl-2-oxo-1,2-oxaphospholidin-2-yl]-2-(4-hydroxy-3,5-dimethylphenyl)ethoxy)pyridine;
2,6-dimethyl-3-(2-[1-oxophosphinan-1-yl]-3-(4-hydroxy-3,5-dimethylphenyl)prop-1-ylamino)pyridine;
2,6-dimethyl-3-(2-[P,P-diprop-1-ylphosphinyl]-2-(4-hydroxy-3,5-dimethylphenyl)cyclohex-1-ylamino)pyridine;
2,6-dimethyl-3-(2-[[4S,5S]-dimethyl-2-oxo-1,3,2-oxazaphospholidin-2-yl]-2-(5-hydroxy-4-methylimidazol-2-yl)ethylamino)pyridine;
2,6-dimethyl-3-(2-[P-ethyl-P-(N-ethylamino)phosphinyl]-2-[6-methoxybenzothien-2-yl]ethylamino)pyridine;
4-methyl-2-(2-[1N-methyl-2-oxo-1,2-azaphosphinan-2-yl]-2-(4-hydroxy-3-methoxyphenyl)ethylamino)imidazole;
4-methyl-2-(2-[1-oxophospholidin-1-yl]-2-(5-hydroxy-4-methylimidazol-2-yl)ethylamino)imidazole;
4-methyl-2-(2-[1-thionophospholidin-1-yl]-2-(5-hydroxy-4-methylimidazol-2-yl)ethylamino)imidazole;
4-methyl-2-(2-[2-oxo-1,3,2-dioxaphospholidin-2-yl]-2-(5-hydroxy-4-methylimidazol-2-yl)ethylamino)imidazole;
4-methyl-2-(3-[5,5-dimethyl-2-oxo-1,3,2-dioxaphosphinan-2-yl]-3-(5-hydroxy-4-methylimidazol-2-yl)prop-1-yl)imidazole;
4-methyl-2-(2-[P-ethyl-P-ethoxyphosphinyl]-3-(5-hydroxy-4-methylimidazol-2-yl)prop-1-ylamino)imidazole;
4-methyl-2-(2-[2-oxo-1,2-oxaphospholidin-2-yl]-3-(5-hydroxy-4-methylimidazol-2-yl)prop-2-en-1-ylamino)imidazole;
4-methyl-2-(2-[[5R]-methyl-2-oxo-1,2-oxaphospholidin-2-yl]-2-(5-[N-methylamino]thiazol-2-yl)ethylamino)imidazole;
4-methyl-2-(2-[1-oxophosphinan-1-yl]-2-[5-(4-hydoxyphenyl)thien-2-yl]ethylamino)imidazole;
4-methyl-2-(2-[P,P-diprop-1-ylphosphinyl]-2-(4-hydroxy-3-methylphenyl)ethylamino)imidazole;
4-methyl-2-(2-[[4S,5S]-dimethyl-2-oxo-1,3,2-oxazaphospholidin-2-yl]-2-(4-hydroxy-3,5-dimethoxyphenyl)ethylamino)imidazole;
4-methyl-2-(2-[P-ethyl-P-(N-ethylamino)phosphinyl]-2-(5-[N-methylamino]thiazol-2-yl)ethylamino)imidazole;
4-methyl-2-(2-[4[R]-methyl-1-thionophospholidin-1-yl]-2-(5-[N-methylamino]thiazol-2-yl)ethylamino)imidazole;
4-methyl-2-(2-[bis(N-ethylamino)phosphinyl]-2-(5-[N-methylamino]thiazol-2-yl)ethylamino)imidazole;
4-methyl-2-(3-[1N-methyl-2-oxo-1,2-azaphosphinan-2-yl]-3-(5-[N-methylamino]thiazol-2-yl)prop-1-yl)imidazole;
4-methyl-2-(2-[5,5-dimethyl-2-oxo-1,3,2-dioxaphosphinan-2-yl]-3-(5-[N-methylamino]thiazol-2-yl)prop-1-ylamino)imidazole;
4-methyl-2-(2-[[4S,5R]-dimethyl-2-oxo-1,3,2-dioxaphospholidin-2-yl]-3-(5-[N-methylamino]thiazol-2-yl)prop-2-en-1-ylamino)imidazole;
4-methyl-2-(2-[2-oxo-1,2-oxaphospholidin-2-yl]-2-(indolin-2-on-5-yl)ethylamino)imidazole;
4-methyl-2-(2-[5,5-dimethyl-2-oxo-1,2-oxaphosphinan-2-yl]-2-[6-methoxybenzothien-2-yl]ethylamino)imidazole;
4-methyl-2-(2-[1-oxophosphinan-1-yl]-2-(4-hydroxy-3-methoxy-5-methylphenyl)ethylamino)imidazole;
4-methyl-2-(2-[[3S,4R]-dimethyl-1-oxophospholidin-1-yl]-2-(5-hydroxy-4-methylimidazol-2-yl)ethylamino)imidazole;
4-methyl-2-(2-[4[S]-methyl-1-thionophospholidin-1-yl]-2-(indolin-2-on-5-yl)ethylamino)imidazole;
4-methyl-2-(2-([4S,5S]-dimethyl-2-oxo 1,3,2-diazaphospholidin-2-yl)-2-(indolin-2-on-5-yl)ethylamino)imidazole;
4-methyl-2-(3-[P,P-diprop-1-ylphosphinyl]-3-(indolin-2-on-5-yl)prop-1-yl)imidazole;
4-methyl-2-(2-[[4S,5S]-dimethyl-2-oxo-1,3,2-oxazaphospholidin-2-yl]-2-(indolin-2-on-5-yl)ethoxy)imidazole;
4-methyl-2-(2-[P-ethyl-P-(N-ethylamino)phosphinyl]-3-(indolin-2-on-5-yl)prop-2-en-1-ylamino)imidazole;
4-methyl-2-(2-[1N-methyl-2-oxo-1,2-azaphosphinan-2-yl]-2-(indolin-2-on-5-yl)cyclohex-1-ylamino)imidazole;
4-methyl-2-(2-[5,5-dimethyl-2-oxo-1,3,2-dioxaphosphinan-2-yl]-2-[5-(hydroxymethylbenzimidazol-2-yl]ethylamino)imidazole;
4-methyl-2-(2-[[4S,5R]-dimethyl-2-oxo-1,3,2-dioxaphospholidin-2-yl]-2-(4-hydroxy-3-methylphenyl)ethylamino)imidazole;
4-methyl-2-(2-[2-oxo-1,2-oxaphospholidin-2-yl]-2-(4-hydroxy-3,5-dimethylphenyl)ethylamino)imidazole;
4-methyl-2-(2-[5,5-dimethyl-2-oxo-1,2-oxaphosphinan-2-yl]-2-(5-[N-methylamino]thiazol-2-yl)ethylamino)imidazole;
4-methyl-2-(2-[3,4-dimethyl-1-thionophospholidin-1-yl]-2-[5-(4-hydoxyphenyl)thien-2-yl]ethylamino)imidazole;
4-methyl-2-(2-([4S]-ethyl-2-oxo-1,3,2-diazaphospholidin-2-yl]-2-[5-(4-hydoxyphenyl)thien-2-yl]ethylamino)imidazole;
4-methyl-2-(3-[1-oxophosphinan-1-yl]-3-[5-(4-hydoxyphenyl)thien-2-yl]prop-1-yl)imidazole;
4-methyl-2-(2-[[3 S,4R]-dimethyl-1-oxophospholidin-1-yl]-2-[5-(4-hydoxyphenyl)thien-2-yl]ethoxy)imidazole;
4-methyl-2-(2-[[4S,5S]-dimethyl-2-oxo-1,3,2-oxazaphospholidin-2-yl]-3-[5-(4-hydoxyphenyl)thien-2-yl]prop-2-en-1-ylamino)imidazole;
4-methyl-2-(2-[P-ethoxy-P-(N-ethylamino)phosphinyl]-2-[5-(4-hydoxyphenyl)thien-2-yl]cyclohex-1-ylamino)imidazole;
4-methyl-2-(2-[1N-methyl-2-oxo-1,2-azaphosphinan-2-yl]-2-(4-hydroxy-3-methoxyphenyl)ethylamino)imidazole;
4-methyl-2-(2-[bis-[P-(N,N-dimethylamino)]phosphinyl]-2-(4-hydroxy-3-methoxy-5-methylphenyl)ethylamino)imidazole;
4-methyl-2-(2-[5,5-dimethyl-2-oxo-1,3,2-dioxaphosphinan-2-yl]-2-(indolin-2-on-5-yl)ethylamino)imidazole;
4-methyl-2-(2-[[4S,5R]-dimethyl-2-oxo-1,3,2-dioxaphospholidin-2-yl]-2-[6-methoxybenzothien-2-yl]ethylamino)imidazole;
4-methyl-2-(2-[P-ethyl (N,N-dimethylamino)phosphinyl]-2-[6-methoxybenzothien-2-yl]ethyl amino)imidazole;
4-methyl-2-(2-[P-ethyl (N-ethylamino)phosphinyl]-2-[6-methoxybenzothien-2-yl]ethylamino)imidazole;
4-methyl-2-(2-[2-oxo-1,2-oxaphospholidin-2-yl]-2-[6-methoxybenzothien-2-yl]ethoxy)imidazole;
4-methyl-2-(2-[5,5-dimethyl-2-oxo-1,2-oxaphosphinan-2-yl]-3-[6-methoxybenzothien-2-yl]prop-1-ylamino)imidazole;
4-methyl-2-(2-[1-oxophosphinan-1-yl]-2-[6-methoxybenzothien-2-yl]cyclohex-1-ylamino)imidazole;
4-methyl-2-(2-[[3S,4R]-dimethyl-1-oxophospholidin-1-yl]-2-[5-(hydroxymethylbenzimidazol-2-yl]ethylamino)imidazole;
4,6-dimethyl-2-(2-[P,P-diprop-1-ylphosphinyl]-2-(4-hydroxy-3-methylphenyl)ethylamino)pyrimidine;
4,6-dimethyl-2-(2-[P-ethoxy-P-(N-ethylamino)phosphinyl]-2-(4-hydroxy-3,5-dimethylphenyl)ethylamino)pyrimidine;
4,6-dimethyl-2-(2-[P-ethyl-P-(N-ethylamino)phosphinyl]-2-(5-[N-methylamino]thiazol-2-yl)ethylamino)ethylamino)pyrimidine;
4,6-dimethyl-2-(2-[bis-[P-(N,N-dimethylamino)]phosphinyl yl]-2-[5-(hydroxymethylbenzimidazol-2-yl]ethylamino)pyrimidine;
4,6-dimethyl-2-(2-[1-thionophospholidin-1-yl]-2-[5-(hydroxymethylbenzimidazol-2-yl]ethylamino)pyrimidine;
4,6-dimethyl-2-(2-[5,5-dimethyl-2-oxo-1,3,2-dioxaphosphinan-2-yl]-2-[5-(hydroxymethylbenzimidazol-2-yl]ethylamino)pyrimidine;
4,6-dimethyl-2-(3-[[4S,5R]-dimethyl-2-oxo-1,3,2-dioxaphospholidin-2-yl]-3-[5-(hydroxymethylbenzimidazol-2-yl]prop-1-yl)pyrimidine;
4,6-dimethyl-2-(2-[2-oxo-1,2-oxaphospholidin-2-yl]-3-[5-(hydroxymethylbenzimidazol-2-yl]prop-1-ylamino)pyrimidine;
4,6-dimethyl-2-(2-[5,5-dimethyl-2-oxo-1,2-oxaphosphinan-2-yl]-3-[5-(hydroxymethylbenzimidazol-2-yl]prop-2-en-1-ylamino)pyrimidine;
4,6-dimethyl-2-(2-[1-oxophosphinan-1-yl]-2-(4-hydroxy-3-methylphenyl)ethylamino)pyrimidine;
4,6-dimethyl-2-(2-[4[R]-methyl-1-thionophospholidin-1-yl]-2-(4-hydroxy-3-methylphenyl)ethylamino)pyrimidine;
4,6-dimethyl-2-(2-([4R]-methyl-2-oxo-1,2-azaphosphinan-2-yl)-2-(4-hydroxy-3-methylphenyl)ethylamino)pyrimidine;
4,6-dimethyl-2-(3-[[3 S,4R]-dimethyl-1-oxophospholidin-1-yl]-3-(4-hydroxy-3-methylphenyl)prop-1-yl)pyrimidine;
4,6-dimethyl-2-(2-[[4S,5S]-dimethyl-2-oxo-1,3,2-oxazaphospholidin-2-yl]-3-(4-hydroxy-3-methylphenyl)prop-1-ylamino)pyrimidine;
4,6-dimethyl-2-(2-[P-ethoxy-P-(N-ethylamino)phosphinyl]-3-(4-hydroxy-3-methylphenyl)prop-2-en-1-ylamino)pyrimidine;
4,6-dimethyl-2-(2-[1N-methyl-2-oxo-1,2-azaphosphinan-2-yl]-2-(4-hydroxy-3-methoxyphenyl)ethylamino)pyrimidine;
4,6-dimethyl-2-(2-[bis-[P-(N,N-dimethylamino)]phosphinyl]-2-(4-hydroxy-3-methoxy-5-methylphenyl)ethylamino)pyrimidine;
4,6-dimethyl-2-(2-[5,5-dimethyl-2-oxo-1,3,2-dioxaphosphinan-2-yl]-2-(indolin-2-on-5-yl)ethylamino)pyrimidine;
4,6-dimethyl-2-(2-[[4S,5R]-dimethyl-2-oxo-1,3,2-dioxaphospholidin-2-yl]-2-[6-methoxybenzothien-2-yl]ethylamino)pyrimidine;
4,6-dimethyl-2-(2-[4[S]-methyl-1-thionophospholidin-1-yl]-2-(4-hydroxy-3-methoxyphenyl)ethylamino)pyrimidine;
4,6-dimethyl-2-(2-[P-ethoxy-P-(N,N-dimethylamino)phosphinyl]-2-(4-hydroxy-3-methoxyphenyl)ethylamino)pyrimidine;
4,6-dimethyl-2-(3-[P-ethyl-P-ethoxyphosphinyl]-3-(4-hydroxy-3-methoxyphenyl)prop-1-yl)pyrimidine;
4,6-dimethyl-2-(2-[2-oxo-1,2-oxaphospholidin-2-yl]-2-(4-hydroxy-3-methoxyphenyl)ethoxy)pyrimidine;
4,6-dimethyl-2-(2-[[5R]-methyl-2-oxo-1,2-oxaphospholidin-2-yl]-3-(4-hydroxy-3-methoxyphenyl)prop-2-en-1-ylamino)pyrimidine;
4,6-dimethyl-2-(2-[1-oxophosphinan-1-yl]-2-(4-hydroxy-3-methoxyphenyl)cyclohex-1-ylamino)pyrimidine;
4,6-dimethyl-2-(2-[P,P-diprop-1-ylphosphinyl]-2-(4-hydroxy-3,5-dimethylphenyl)ethylamino)pyrimidine;
4,6-dimethyl-2-(2-[[4S,5S]-dimethyl-2-oxo-1,3,2-oxazaphospholidin-2-yl]-2-(5-[N-methylamino]thiazol-2-yl)ethylamino)ethylamino)pyrimidine;
4,6-dimethyl-2-(2-[1N-methyl-2-oxo-1,2-azaphosphinan-2-yl]-2-[5-(hydroxymethylbenzimidazol-2-yl]ethylamino)pyrimidine;
4,6-dimethyl-2-(2-[2-oxo-1,2-azaphospholidin-2-yl]-2-(4-hydroxy-3-methylphenyl) ethyl amino)pyrimidine;
4,6-dimethyl-2-(2-[3,4-dimethyl-1-thionophospholidin-1-yl]-2-(4-hydroxy-3,5-dimethoxyphenyl)ethylamino)pyrimidine;
4,6-dimethyl-2-(2-([4S,5S]-dimethyl-2-oxo-1,3,2-oxazaphospholidin-2-yl)-2-(4-hydroxy-3,5-dimethoxyphenyl)ethylamino)pyrimidine;
4,6-dimethyl-2-(3-[5,5-dimethyl-2-oxo-1,3,2-dioxaphosphinan-2-yl]-3-(4-hydroxy-3,5-dimethoxyphenyl)prop-1-yl)pyrimidine;
4,6-dimethyl-2-(2-[2-oxo-1,2-azaphospholidin-2-yl]-2-(4-hydroxy-3,5-dimethoxyphenyl)ethoxy)pyrimidine;
4,6-dimethyl-2-(2-[bis-[P-(N,N-dimethylamino)]phosphinyl]-3-(4-hydroxy-3,5-dimethoxyphenyl)prop-2-en-1-ylamino)pyrimidine;
4,6-dimethyl-2-(2-[3,4-dimethyl-1-thionophospholidin-1-yl]-2-(4-hydroxy-3,5-dimethoxyphenyl)cyclohex-1-ylamino)pyrimidine;
4,6-dimethyl-2-(2-[P-ethyl-P-ethoxyphosphinyl]-2-(5-hydroxy-4-methylimidazol-2-yl)ethylamino)pyrimidine;
4,6-dimethyl-2-(2-[2-oxo-1,2-oxaphospholidin-2-yl]-2-(indolin-2-on-5-yl)ethylamino)pyrimidine;
4,6-dimethyl-2-(2-[[5R]-methyl-2-oxo-1,2-oxaphospholidin-2-yl]-2-(4-hydroxy-3-methoxyphenyl)ethylamino)pyrimidine;
4,6-dimethyl-2-(2-[1-oxophosphinan-1-yl]-2-(4-hydroxy-3-methoxy-5-methylphenyl)ethylamino)pyrimidine;
4,6-dimethyl-2-(2-([4S]-ethyl-2-oxo-1,3,2-oxazaphospholidin-2-yl)-2-(4-hydroxy-3-methoxy-5-methylphenyl)ethylamino)pyrimidine;
4,6-dimethyl-2-(2-([4R]methyl-2-oxo-1,3,2-oxazaphosphinan-2-yl)-2-(4-hydroxy-3-methoxy-5-methylphenyl)ethylamino)pyrimidine;
4,6-dimethyl-2-(2-[[3S,4R]-dimethyl-1-oxophospholidin-1-yl]-2-(4-hydroxy-3-methoxy-5-methylphenyl)ethoxy)pyrimidine;
4,6-dimethyl-2-(2-[P,P-diprop-1-ylphosphinyl]-3-(4-hydroxy-3-methoxy-5-methylphenyl)prop-1-ylamino)pyrimidine;
4,6-dimethyl-2-(2-[P-ethoxy-P-(N-ethylamino)phosphinyl]-2-(4-hydroxy-3-methoxy-5-methylphenyl)cyclohex-1-ylamino)pyrimidine;
4,6-dimethyl-2-(2-[2-oxo-1,3,2-dioxaphospholidin-2-yl]-2-(4-hydroxy-3,5-dimethylphenyl)ethylamino)pyrimidine;
4,6-dimethyl-2-(2-[[4S,5R]-dimethyl-2-oxo-1,3,2-dioxaphospholidin-2-yl]-2-[5-(4-hydoxyphenyl)thien-2-yl]ethylamino)pyrimidine;
4,6-dimethyl-2-(2-[P-ethyl-P-ethoxyphosphinyl-1-oxophosphinan-1-yl]-2-[5-(hydroxymethylbenzimidazol-2-yl]ethylamino)pyrimidine;
3-(2-[5,5-dimethyl-2-oxo-1,2-oxaphosphinan-2-yl]-2-(4-hydroxy-3,5-dimethoxyphenyl)ethylamino)quinoline;
3-(2-[[5R]-methyl-2-oxo-1,2-oxaphospholidin-2-yl]-2-(4-hydroxy-3-methoxy-5-methylphenyl)ethylamino)quinoline;
3-(2-([4R]methyl-2-oxo-1,3,2-oxazaphosphinan-2-yl)-2-(4-hydroxy-3,5-dimethylphenyl)ethylamino)quinoline;
3-(2-([4S]-ethyl-2-oxo-1,3,2-oxazaphospholidin-2-yl)-2-(4-hydroxy-3,5-dimethylphenyl)ethylamino)quinoline;
3-(3-[1-oxophosphinan-1-yl]-3-(4-hydroxy-3,5-dimethylphenyl)prop-1-yl)quinoline;
3-(2-[[3S,4R]-dimethyl-1-oxophospholidin-1-yl]-2-(4-hydroxy-3,5-dimethylphenyl)ethoxy)quinoline;
3-(2-[[4S,5 S]-dimethyl-2-oxo-1,3,2-oxazaphospholidin-2-yl]-3-(4-hydroxy-3,5-dimethylphenyl)prop-2-en-1-ylamino)quinoline;
3-(2-[P-ethoxy-P-(N-ethylamino)phosphinyl]-2-(4-hydroxy-3,5-dimethylphenyl)cyclohex-1-ylamino)quinoline;
3-(2-[1N-methyl-2-oxo-1,2-azaphosphinan-2-yl]-2-(indolin-2-on-5-yl)ethylamino)quinoline;
3-(2-[2-oxo-1,3,2-dioxaphospholidin-2-yl]-2-[6-methoxybenzothien-2-yl]ethylamino)quinoline;
3-(2-[[4S,5R]-dimethyl-2-oxo-1,3,2-dioxaphospholidin-2-yl]-2-(4-hydroxy-3-methoxy-5-methylphenyl)ethylamino)quinoline;
3-(2-[4[P-ethyl-P-ethoxyphosphinyl]-2-(5-hydroxy-4-methylimidazol-2-yl)ethylamino)quinoline;
3-(2-[3,5,5-trimethyl-2-oxo-1,3,2-oxazaphosphinan-2-yl]-2-(5-hydroxy-4-methylimidazol-2-yl)ethylamino)quinoline;
3-(2-([4S,5S]-dimethyl-2-oxo-1,3,2-oxazaphospholidin-2-yl)-2-(5-hydroxy-4-methylimidazol-2-yl)ethylamino)quinoline;
3-(3-[5,5-dimethyl-2-oxo-1,2-oxaphosphinan-2-yl]-3-(5-hydroxy-4-methylimidazol-2-yl)prop-1-yl)quinoline;
3-(2-[[5R]-methyl-2-oxo-1,2-oxaphospholidin-2-yl]-2-(5-hydroxy-4-methylimidazol-2-yl)ethoxy)quinoline;
3-(2-[[3 S,4R]-dimethyl-1-oxophospholidin-1-yl]-3-(5-hydroxy-4-methylimidazol-2-yl)prop-2-en-1-ylamino)quinoline;
3-(2-[PP-diprop-1-ylphosphinyl]-2-(5-hydroxy-4-methylimidazol-2-yl)cyclohex-1-ylamino)quinoline;
3-(2-[P-ethoxy-P-(N-ethylamino)phosphinyl]-2-[5-(4-hydoxyphenyl)thien-2-yl]ethylamino)quinoline;
3-(2-[P-ethyl-P-(N-ethylamino)phosphinyl 2-[5-(hydroxymethylbenzimidazol-2-yl]ethylamino)quinoline;
3-(2-[2-oxo-1,2-azaphospholidin-2-yl]-2-(4-hydroxy-3,5-dimethoxyphenyl)ethylamino)quinoline;
3-(2-[2-oxo-1,3,2-dioxaphospholidin-2-yl]-2-(4-hydroxy-3,5-dimethylphenyl)ethylamino)quinoline;
3-(2-[4[S]-methyl-1-thionophospholidin-1-yl]-2-(5-[N-methylamino]thiazol-2-yl)ethylamino)quinoline;
3-(2-[P-(N-ethylamino)-P-propoxyphosphinyl]-2-(5-[N-methylamino]thiazol-2-yl)ethylamino)quinoline;
3-(2-[2-oxo-1,2-oxaphospholidin-2-yl]-2-(5-[N-methylamino]thiazol-2-yl)ethylamino)quinoline;
3-(3-[4[S]-methyl-1-thionophospholidin-1-yl]-3-(5-[N-methylamino]thiazol-2-yl)prop-1-yl)quinoline;
3-(2-[2-oxo-1,2-azaphospholidin-2-yl]-3-(5-[N-methylamino]thiazol-2-yl)prop-1-ylamino)quinoline;
3-(2-[4[S]-methyl-1-thionophospholidin-1-yl]-3-(5-[N-methylamino]thiazol-2-yl)prop-2-en-1-ylamino)quinoline;
3-(2-[[4S,5R]-dimethyl-2-oxo-1,3,2-dioxaphospholidin-2-yl]-2-(indolin-2-on-5-yl)ethylamino)quinoline;
3-(2-[P-ethyl-P-ethoxyphosphinyl]-2-[6-methoxybenzothien-2-yl]ethylamino)quinoline;
3-(2-[[5R]-methyl-2-oxo-1,2-oxaphospholidin-2-yl]-2-(4-hydroxy-3-methoxy-5-methylphenyl)ethylamino)quinoline;
3-(2-[1-oxophosphinan-1-yl]-2-(5-hydroxy-4-methylimidazol-2-yl)ethylamino)quinoline;
3-(2-[3,4-dimethyl-1-thionophospholidin-1-yl]-2-(indolin-2-on-5-yl)ethylamino)quinoline;
3-(2-([4R]-methyl-2-oxo-1,2-azaphosphinan-2-yl)-2-(indolin-2-on-5-yl)ethylamino)quinoline;
3-(2-[[5S]-methyl-2-oxo-1,2-oxaphospholidin-2-yl]-2-(indolin-2-on-5-yl)ethylamino)quinoline;
3-(2-[PP-diprop-1-ylphosphinyl]-2-(indolin-2-on-5-yl)prop-1-yl)quinoline;
3-(2-[2-oxo-1,3,2-dioxaphospholidin-2-yl]-3-(indolin-2-on-5-yl)prop-1-ylamino)quinoline;
3-(2-[5,5-dimethyl-2-oxo-1,3,2-dioxaphosphinan-2-yl]-3-(indolin-2-on-5-yl)prop-2-en-1-ylamino)quinoline;
3-(2-[P-ethyl-P-ethoxyphosphinyl]-2-[5-(4-hydoxyphenyl)thien-2-yl]ethylamino)quinoline;
3-(2-[2-oxo-1,2-oxaphospholidin-2-yl]-2-[5-(hydroxymethylbenzimidazol-2-yl]ethylamino)quinoline;
3-(2-[[5R]-methyl-2-oxo-1,2-oxaphospholidin-2-yl]-2-(4-hydroxy-3,5-dimethoxyphenyl)ethylamino)quinoline;
3-(2-[1-thionphosphinan-1-yl]-2-(4-hydroxy-3,5-dimethoxyphenyl)ethylamino)quinoline;
3-(2-([3S,4S]-dimethyl-2-oxo-1,2-azaphospholidin-2-yl)-2-(4-hydroxy-3,5-dimethoxyphenyl)ethylamino)quinoline;
3-(2-[4,5-dimethyl-2-oxo-1,2-oxaphospholidin-2-yl]-2-(4-hydroxy-3,5-dimethoxyphenyl)ethylamino)quinoline;
3-(2-[P,P-diprop-1-ylphosphinyl]-2-(4-hydroxy-3,5-dimethoxyphenyl)ethoxy)quinoline;
3-(2-[[4S,5S]-dimethyl-2-oxo-1,3,2-oxazaphospholidin-2-yl]-3-(4-hydroxy-3,5-dimethoxyphenyl)prop-2-en-1-ylamino)quinoline;
3-(2-[P-ethyl-P-(N-ethylamino)phosphinyl]-2-(4-hydroxy-3,5-dimethoxyphenyl)cyclohex-1-ylamino)quinoline;
3-(2-[1N-methyl-2-oxo-1,2-azaphosphinan-2-yl]-2-(4-hydroxy-3-methoxy-5-methylphenyl)ethylamino)quinoline;
3-(2-[2-oxo-1,3,2-dioxaphospholidin-2-yl]-2-(indolin-2-on-5-yl)ethyla,mino)quinoline;
3-(2-[5,5-dimethyl-2-oxo-1,3,2-dioxaphosphinan-2-yl]-2-[6-methoxybenzothien-2-yl]ethylamino)quinoline;
5-methyl-2-(2-[P-ethyl-P-ethoxyphosphinyl]-2-(4-hydroxy-3-methoxy-5-methylphenyl)ethylamino)pyrazine;
5-methyl-2-(2-[2-oxo-1,2-oxaphospholidin-2-yl]-2-(5-hydroxy-4-methylimidazol-2-yl)ethylamino)pyrazine;
5-methyl-2-(2-[1-thionophospholidin-1-yl]-2-[5-(4-hydoxyphenyl)thien-2-yl]ethylamino)pyrazine;
5-methyl-2-(2-[P-ethyl (N-ethylamino)phosphinyl]-2-[5-(4-hydoxyphenyl)thien-2-yl]ethylamino)pyrazine;
5-methyl-2-(2-[2-oxo-1,2-oxaphosphinan-2-yl]-2-[5-(4-hydoxyphenyl)thien-2-yl]ethylamino)pyrazine;
5-methyl-3-(2-[5,5-dimethyl-2-oxo-1,2-oxaphosphinan-2-yl]-3-[5-(4-hydoxyphenyl)thien-2-yl]prop-1-yl)pyrazine;
5-methyl-2-(2-[1-oxophosphinan-1-yl]-3-[5-(4-hydoxyphenyl)thien-2-yl]prop-1-ylamino)pyrazine;
5-methyl-2-(2-[[3 S,4R]-dimethyl-1-oxophospholidin-1-yl]-3-[5-(4-hydoxyphenyl)thien-2-yl]prop-2-en-1-ylamino)pyrazine;
5-methyl-2-(2-[[4S,5S]-dimethyl-2-oxo-1,3,2-oxazaphospholidin-2-yl]-2-[5-(4-hydoxyphenyl)thien-2-yl]ethylamino)pyrazine;
5-methyl-2-(2-[P-ethoxy-P-(N-ethylamino)phosphinyl]-2-[6-methoxybenzothien-2-yl]ethylamino)pyrazine;
5-methyl-2-(2-[2-oxo-1,3,2-dioxaphospholidin-2-yl]-2-(4-hydroxy-3-methoxyphenyl)ethylamino)pyrazine;
5-methyl-2-(2-[5,5-dimethyl-2-oxo-1,3,2-dioxaphosphinan-2-yl]-2-(4-hydroxy-3-methoxy-5-methylphenyl)ethylamino)pyrazine;
5-methyl-2-(2-[P-ethyl-P-ethoxyphosphinyl]-2-(indolin-2-on-5-yl)ethylamino)pyrazine;
5-methyl-2-(2-[2-oxo-1,2-oxaphospholidin-2-yl]-2-[6-methoxybenzothien-2-yl]ethylamino)pyrazine;
5-methyl-2-(2-([4R]-methyl-2-oxo-1,3,2-diazaphosphinan-2-yl)-2-[6-methoxybenzothien-2-yl]ethylamino)pyrazine;
5-methyl-2-(2-([4S]-ethyl-2-oxo-1,3,2-diazaphospholidin-2-yl)-2-[6-methoxybenzothien-2-yl]ethylamino)pyrazine;
5-methyl-3-(2-[5,5-dimethyl-2-oxo-1,2-oxaphosphinan-2-yl]-3-[6-methoxybenzothien-2-yl]prop-1-yl)pyrazine;
5-methyl-2-(2-[[5R]-methyl-2-oxo-1,2-oxaphospholidin-2-yl]-2-[6-methoxybenzothien-2-yl]ethoxy)pyrazine;
5-methyl-2-(2-[4[R]-methyl-1-oxophospholidin-1-yl]-3-[6-methoxybenzothien-2-yl]prop-2-en-1-ylamino)pyrazine;
5-methyl-2-(2-[PP-diprop-1-ylphosphinyl]-2-[6-methoxybenzothien-2-yl]cyclohex-1-ylamino)pyrazine;
5-methyl-2-(2-[P-ethoxy-P-(N-ethylamino)phosphinyl]-2-(4-hydroxy-3-methylphenyl)ethylamino)pyrazine;
5-methyl-2-(2-[P-ethyl-P-(N-ethylamino)phosphinyl]-2-(4-hydroxy-3,5-dimethoxyphenyl)ethylamino)pyrazine;
5-methyl-2-(2-[2-oxo-1,2-azaphospholidin-2-yl]-2-(5-[N-methylamino]thiazol-2-yl)ethylamino)pyrazine;
5-methyl-2-(2-[2-oxo-1,3,2-dioxaphospholidin-2-yl]-2-[5-(4-hydoxyphenyl)thien-2-yl]ethylamino)pyrazine;
5-methyl-2-(2-[1,3,5,5-tetramethyl-2-oxo-1,3,2-diazaphosphinan-2-yl]-2-[5-(hydroxymethylbenzimidazol-2-yl]ethylamino)pyrazine;
5-methyl-2-(2-([4S,5S]-dimethyl-2-oxo 1,3,2-diazaphospholidin-2-yl)-2-[5-(hydroxymethylbenzimidazol-2-yl]ethylamino)pyrazine;
5-methyl-3-(2-[[4S,5R]-dimethyl-2-oxo-1,3,2-dioxaphospholidin-2-yl]-3-[5-(hydroxymethylbenzimidazol-2-yl]prop-1-yl)pyrazine;
5-methyl-2-(2-[P-ethyl-P-ethoxyphosphinyl]-2-[5-(hydroxymethylbenzimidazol-2-yl]ethoxy)pyrazine;
5-methyl-2-(2-[5,5-dimethyl-2-oxo-1,2-oxaphosphinan-2-yl]-3-[5-(hydroxymethylbenzimidazol-2-yl]prop-2-en-1-ylamino)pyrazine;
5-methyl-2-(2-[[5R]-methyl-2-oxo-1,2-oxaphospholidin-2-yl]-2-[5-(hydroxymethylbenzimidazol-2-yl]cyclohex-1-ylamino)pyrazine;
5-methyl-2-(2-[3,4-dimethyl-1-thionophospholidin-1-yl]-2-(4-hydroxy-3-methylphenyl)ethylamino)pyrazine;
5-methyl-2-(2-[bis(N-ethylamino)phosphinyl]-2-(4-hydroxy-3-methylphenyl)ethylamino)pyrazine;
5-methyl-2-(2-[4[R]-methyl-1-oxophospholidin-1-yl]-2-(4-hydroxy-3-methylphenyl)ethylamino)pyrazine;
5-methyl-3-(2-[PP-diprop-1-ylphosphinyl]-3-(4-hydroxy-3-methylphenyl)prop-1-yl)pyrazine;
5-methyl-2-(2-[P-ethoxy-P-(N-ethylamino)phosphinyl]-3-(4-hydroxy-3-methylphenyl)prop-1-ylamino)pyrazine;
5-methyl-2-(2-[P-ethyl-P-(N-ethylamino)phosphinyl]-3-(4-hydroxy-3-methylphenyl)prop-2-en-1-ylamino)pyrazine;
5-methyl-2-(2-[5,5-dimethyl-2-oxo-1,3,2-dioxaphosphinan-2-yl]-2-(4-hydroxy-3-methoxyphenyl)ethylamino)pyrazine;
5-methyl-2-(2-[[4S,5R]-dimethyl-2-oxo-1,3,2-dioxaphospholidin-2-yl]-2-(4-hydroxy-3-methoxy-5-methylphenyl)ethylamino)pyrazine;
5-methyl-2-(2-[2-oxo-1,2-oxaphospholidin-2-yl]-2-(indolin-2-on-5-yl)ethylamino)pyrazine;
5-methyl-2-(2-[5,5-dimethyl-2-oxo-1,2-oxaphosphinan-2-yl]-2-[5-(hydroxymethylbenzimidazol-2-yl]ethylamino)pyrazine;
5-methyl-2-(2-[1-thionophosphinan-1-yl]-2-(4-hydroxy-3-methoxyphenyl)ethylamino)pyrazine;
5-methyl-2-(2-[1-oxophosphinan-1-yl]-2-(4-hydroxy-3-methoxyphenyl)ethylamino)pyrazine;
5-methyl-2-(2-[PP-diprop-1-ylphosphinyl]-2-(4-hydroxy-3-methoxyphenyl)ethylamino)pyrazine;
5-methyl-3-(2-[[4S,5S]-dimethyl-2-oxo-1,3,2-oxazaphospholidin-2-yl]-3-(4-hydroxy-3-methoxyphenyl)prop-1-yl)pyrazine;
5-methyl-2-(2-[P-ethyl-P-(N-ethylamino)phosphinyl]-3-(4-hydroxy-3-methoxyphenyl)prop-1-ylamino)pyrazine;
5-methyl-2-(2-[1N-methyl-2-oxo-1,2-azaphosphinan-2-yl]-3-(4-hydroxy-3-methoxyphenyl)prop-2-en-1-ylamino)pyrazine;
5-methyl-2-(2-[1N,3N-dimethyl-2-oxo-1,3,2-diazaphosphinan-2-yl]-2-(4-hydroxy-3,5-dimethoxyphenyl)ethylamino)pyrazine;
5-methyl-2-(2-[1-oxophosphinan-1-yl]-2-(4-hydroxy-3,5-dimethylphenyl)ethylamino)pyrazine;
5-methyl-2-(2-[1N,3N-dimethyl-2-oxo-1,3,2-diazaphosphinan-2-yl]-2-[5-(4-hydoxyphenyl)thien-2-yl]ethylamino)pyrazine;
5-methyl-2-(2-[1-oxophosphinan-1-yl]-2-[5-(hydroxymethylbenzimidazol-2-yl]ethylamino)pyrazine;
2,6-dimethyl-3-(2-[2-oxo-1,3,2-dioxaphospholidin-2-yl]-2-(4-hydroxy-3-methoxy-5-methylphenyl)ethylamino)pyridine;
2,6-dimethyl-3-(2-[5,5-dimethyl-2-oxo-1,3,2-dioxaphosphinan-2-yl]-2-(4-hydroxy-3-methoxy-5-methylphenyl)ethylamino)pyridine;
2,6-dimethyl-3-(2-([4S,5 S]-dimethyl-2-oxo-1,3,2-oxazaphospholidin-2-yl)-2-(4-hydroxy-3-methoxy-5-methylphenyl)ethylamino)pyridine;
2,6-dimethyl-3-(2-[3N,5,5-trimethyl-2-oxo-1,3,2-oxazaphosphinan-2-yl]-2-(4-hydroxy-3-methoxy-5-methylphenyl)ethylamino)pyridine;
2,6-dimethyl-3-(2-[5,5-dimethyl-2-oxo-1,3,2-diazaphosphinan-2-yl]-2-(4-hydroxy-3-methoxy-5-methylphenyl)ethylamino)pyridine;
2,6-dimethyl-3-(2-[bis-P-(N,N-dimethylamino)phosphinyl]-2-(4-hydroxy-3-methoxy-5-methylphenyl)ethylamino)pyridine;
2,6-dimethyl-3-(2-[bis-P-(N-ethylamino)phosphinyl]-2-(4-hydroxy-3-methoxy-5-methylphenyl)ethylamino)pyridine;
2,6-dimethyl-3-(2-[P-ethoxy-P-(N-ethylamino)phosphinyl]-2-(4-hydroxy-3-methoxy-5-methylphenyl)ethylamino)pyridine;
3,5-dimethyl-2-(2-[2-oxo-1,3,2-dioxaphospholidin-2-yl]-2-(4-hydroxy-3-methoxy-5-methylphenyl)ethylamino)pyrazine;
3,5-dimethyl-2-(2-[5,5-dimethyl-2-oxo-1,3,2-dioxaphosphinan-2-yl]-2-(4-hydroxy-3-methoxy-5-methylphenyl)ethylamino)pyrazine;
3,5-dimethyl-2-(2-([4S,5S]-dimethyl-2-oxo-1,3,2-oxazaphospholidin-2-yl)-2-(4-hydroxy-3-methoxy-5-methylphenyl)ethylamino)pyrazine;
3,5-dimethyl-2-(2-[3N,5,5-trimethyl-2-oxo-1,3,2-oxazaphosphinan-2-yl]-2-(4-hydroxy-3-methoxy-5-methylphenyl)ethylamino)pyrazine;
3,5-dimethyl-2-(2-[5,5-dimethyl-2-oxo-1,3,2-diazaphosphinan-2-yl]-2-(4-hydroxy-3-methoxy-5-methylphenyl)ethylamino)pyrazine;
3,5-dimethyl-2-(2-[bis-P-(N,N-dimethylamino)phosphinyl]-2-(4-hydroxy-3-methoxy-5-methylphenyl)ethylamino)pyrazine;
3,5-dimethyl-2-(2-[bis-P-(N-ethylamino)phosphinyl]-2-(4-hydroxy-3-methoxy-5-methylphenyl)ethylamino)pyrazine;
3,5-dimethyl-2-(2-[P-ethoxy-P-(N-ethylamino)phosphinyl]-2-(4-hydroxy-3-methoxy-5-methylphenyl)ethylamino)pyrazine;
2,4-dimethyl-5-(2-[2-oxo-1,3,2-dioxaphospholidin-2-yl]-2-(4-hydroxy-3-methoxy-5-methylphenyl)ethylamino)pyrimidine;
2,4-dimethyl-5-(2-[5,5-dimethyl-2-oxo-1,3,2-dioxaphosphinan-2-yl]-2-(4-hydroxy-3-methoxy-5-methylphenyl)ethylamino)pyrimidine;
2,4-dimethyl-5-(2-([4S,5S]-dimethyl-2-oxo-1,3,2-oxazaphospholidin-2-yl)-2-(4-hydroxy-3-methoxy-5-methylphenyl)ethylamino)pyrimidine;
2,4-dimethyl-5-(2-[3N,5,5-trimethyl-2-oxo-1,3,2-oxazaphosphinan-2-yl]-2-(4-hydroxy-3-methoxy-5-methylphenyl)ethylamino)pyrimidine;
2,4-dimethyl-5-(2-[5,5-dimethyl-2-oxo-1,3,2-diazaphosphinan-2-yl]-2-(4-hydroxy-3-methoxy-5-methylphenyl)ethylamino)pyrimidine;
2,4-dimethyl-5-(2-[bis-P-(N,N-dimethylamino)phosphinyl]-2-(4-hydroxy-3-methoxy-5-methylphenyl)ethylamino)pyrimidine;
2,4-dimethyl-5-(2-[bis-P-(N-ethylamino)phosphinyl]-2-(4-hydroxy-3-methoxy-5-methylphenyl)ethylamino)pyrimidine;
2,4-dimethyl-5-(2-[P-ethoxy-P-(N-ethylamino)phosphinyl]-2-(4-hydroxy-3-methoxy-5-methylphenyl)ethylamino)pyrimidine;
2-methyl-5-(2-[2-oxo-1,3,2-dioxaphospholidin-2-yl]-2-(4-hydroxy-3-methoxy-5-methylphenyl)ethylamino)thiazole;
2-methyl-5-(2-[5,5-dimethyl-2-oxo-1,3,2-dioxaphosphinan-2-yl]-2-(4-hydroxy-3-methoxy-5-methylphenyl)ethylamino)thiazole;
2-methyl-5-(2-([4S,5S]-dimethyl-2-oxo-1,3,2-oxazaphospholidin-2-yl)-2-(4-hydroxy-3-methoxy-5-methylphenyl)ethylamino)thiazole;
2-methyl-5-(2-[3N,5,5-trimethyl-2-oxo-1,3,2-oxazaphosphinan-2-yl]-2-(4-hydroxy-3-methoxy-5-methylphenyl)ethylamino)thiazole;
2-methyl-5-(2-[5,5-dimethyl-2-oxo-1,3,2-diazaphosphinan-2-yl]-2-(4-hydroxy-3-methoxy-5-methylphenyl)ethylamino)thiazole;
2-methyl-5-(2-[bis-P-(N,N-dimethylamino)phosphinyl]-2-(4-hydroxy-3-methoxy-5-methylphenyl)ethyl amino)thiazole;
2-methyl-5-(2-[bis-P-(N-ethylamino)phosphinyl]-2-(4-hydroxy-3-methoxy-5-methylphenyl)ethylamino)thiazole; and
2-methyl-5-(2-[P-ethoxy-P-(N-ethylamino)phosphinyl]-2-(4-hydroxy-3-methoxy-5-methylphenyl)ethylamino)thiazole.

7. A compound according to claim 5, wherein the compound is selected from the group consisting of:
2,6-dimethyl-3-(2-[2-oxo-1,3,2-dioxaphospholidin-2-yl]-2-(4-hydroxy-3-methoxy-5-methylphenyl)ethylamino)pyridine;
2,6-dimethyl-3-(2-[5,5-dimethyl-2-oxo-1,3,2-dioxaphosphinan-2-yl]-2-(4-hydroxy-3-methoxy-5-methylphenyl)ethylamino)pyridine;
2,6-dimethyl-3-(2-([4S,5S]-dimethyl-2-oxo-1,3,2-oxazaphospholidin-2-yl)-2-(4-hydroxy-3-methoxy-5-methylphenyl)ethylamino)pyridine;
2,6-dimethyl-3-(2-[3N,5,5-trimethyl-2-oxo-1,3,2-oxazaphosphinan-2-yl]-2-(4-hydroxy-3-methoxy-5-methylphenyl)ethylamino)pyridine;
2,6-dimethyl-3-(2-[5,5-dimethyl-2-oxo-1,3,2-diazaphosphinan-2-yl]-2-(4-hydroxy-3-methoxy-5-methylphenyl)ethylamino)pyridine;
2,6-dimethyl-3-(2-[bis-P-(N,N-dimethylamino)phosphinyl]-2-(4-hydroxy-3-methoxy-5-methylphenyl)ethylamino)pyridine;
2,6-dimethyl-3-(2-[bis-P-(N-ethylamino)phosphinyl]-2-(4-hydroxy-3-methoxy-5-methylphenyl)ethylamino)pyridine;
2,6-dimethyl-3-(2-[P-ethoxy-P-(N-ethylamino)phosphinyl]-2-(4-hydroxy-3-methoxy-5-methylphenyl)ethylamino)pyridine;
3,5-dimethyl-2-(2-[2-oxo-1,3,2-dioxaphospholidin-2-yl]-2-(4-hydroxy-3-methoxy-5-methylphenyl)ethylamino)pyrazine;
3,5-dimethyl-2-(2-[5,5-dimethyl-2-oxo-1,3,2-dioxaphosphinan-2-yl]-2-(4-hydroxy-3-methoxy-5-methylphenyl)ethylamino)pyrazine;
3,5-dimethyl-2-(2-([4S,5S]-dimethyl-2-oxo-1,3,2-oxazaphospholidin-2-yl)-2-(4-hydroxy-3-methoxy-5-methylphenyl)ethylamino)pyrazine;
3,5-dimethyl-2-(2-[3N,5,5-trimethyl-2-oxo-1,3,2-oxazaphosphinan-2-yl]-2-(4-hydroxy-3-methoxy-5-methylphenyl)ethylamino)pyrazine;
3,5-dimethyl-2-(2-[5,5-dimethyl-2-oxo-1,3,2-diazaphosphinan-2-yl]-2-(4-hydroxy-3-methoxy-5-methylphenyl)ethylamino)pyrazine;
3,5-dimethyl-2-(2-[bis-P-(N,N-dimethylamino)phosphinyl]-2-(4-hydroxy-3-methoxy-5-methylphenyl)ethylamino)pyrazine;
3,5-dimethyl-2-(2-[bis-P-(N-ethylamino)phosphinyl]-2-(4-hydroxy-3-methoxy-5-methylphenyl)ethylamino)pyrazine;
3,5-dimethyl-2-(2-[P-ethoxy-P-(N-ethylamino)phosphinyl]-2-(4-hydroxy-3-methoxy-5-methylphenyl)ethylamino)pyrazine;
2,4-dimethyl-5-(2-[2-oxo-1,3,2-dioxaphospholidin-2-yl]-2-(4-hydroxy-3-methoxy-5-methylphenyl)ethylamino)pyrimidine;
2,4-dimethyl-5-(2-[5,5-dimethyl-2-oxo-1,3,2-dioxaphosphinan-2-yl]-2-(4-hydroxy-3-methoxy-5-methylphenyl)ethylamino)pyrimidine;
2,4-dimethyl-5-(2-([4S,5 S]-dimethyl-2-oxo-1,3,2-oxazaphospholidin-2-yl)-2-(4-hydroxy-3-methoxy-5-methylphenyl)ethylamino)pyrimidine;
2,4-dimethyl-5-(2-[3N,5,5-trimethyl-2-oxo-1,3,2-oxazaphosphinan-2-yl]-2-(4-hydroxy-3-methoxy-5-methylphenyl)ethylamino)pyrimidine;
2,4-dimethyl-5-(2-[5,5-dimethyl-2-oxo-1,3,2-diazaphosphinan-2-yl]-2-(4-hydroxy-3-methoxy-5-methylphenyl)ethylamino)pyrimidine;
2,4-dimethyl-5-(2-[bis-P-(N,N-dimethylamino)phosphinyl]-2-(4-hydroxy-3-methoxy-5-methylphenyl)ethylamino)pyrimidine;
2,4-dimethyl-5-(2-[bis-P-(N-ethylamino)phosphinyl]-2-(4-hydroxy-3-methoxy-5-methylphenyl)ethylamino)pyrimidine;
2,4-dimethyl-5-(2-[P-ethoxy-P-(N-ethylamino)phosphinyl]-2-(4-hydroxy-3-methoxy-5-methylphenyl)ethylamino)pyrimidine;
2-methyl-5-(2-[2-oxo-1,3,2-dioxaphospholidin-2-yl]-2-(4-hydroxy-3-methoxy-5-methylphenyl)ethylamino)thiazole;
2-methyl-5-(2-[5,5-dimethyl-2-oxo-1,3,2-dioxaphosphinan-2-yl]-2-(4-hydroxy-3-methoxy-5-methylphenyl)ethylamino)thiazole;
2-methyl-5-(2-([4S,5S]-dimethyl-2-oxo-1,3,2-oxazaphospholidin-2-yl)-2-(4-hydroxy-3-methoxy-5-methylphenyl)ethylamino)thiazole;
2-methyl-5-(2-[3N,5,5-trimethyl-2-oxo-1,3,2-oxazaphosphinan-2-yl]-2-(4-hydroxy-3-methoxy-5-methylphenyl)ethylamino)thiazole;
2-methyl-5-(2-[5,5-dimethyl-2-oxo-1,3,2-diazaphosphinan-2-yl]-2-(4-hydroxy-3-methoxy-5-methylphenyl)ethylamino)thiazole;
2-methyl-5-(2-[bis-P-(N,N-dimethylamino)phosphinyl]-2-(4-hydroxy-3-methoxy-5-methylphenyl)ethylamino)thiazole;
2-methyl-5-(2-[bis-P-(N-ethylamino)phosphinyl]-2-(4-hydroxy-3-methoxy-5-methylphenyl)ethylamino)thiazole; and
2-methyl-5-(2-[P-ethoxy-P-(N-ethylamino)phosphinyl]-2-(4-hydroxy-3-methoxy-5-methylphenyl)ethylamino)thiazole.

8. A pharmaceutical composition comprising a compound according to claim 1 and a pharmaceutically acceptable carrier, excipient, solvent, adjuvant or diluent.

9. A pharmaceutical composition comprising a compound according to claim 6 and a pharmaceutically acceptable carrier, excipient, solvent, adjuvant or diluent.

10. A pharmaceutical composition comprising a compound according to claim 7 and a pharmaceutically acceptable carrier, excipient, solvent, adjuvant or diluent.

11. A method of regulating Lp(a) or LDL cholesterol levels in a mammal comprising administering to a mammal an effective amount of a pharmaceutical composition according to claim 8.

12. A method according to claim 11, wherein the mammal is a human.

13. A method according to claim 11, wherein the pharmaceutical composition is administered in a dosage of between about 0.1 mg to about 5000 mg per day.

14. A method according to claim 11, wherein the pharmaceutical composition is administered in a dosage of between about 50 mg to about 500 mg per day.

15. A method according to claim 11, wherein the pharmaceutical composition is administered orally.

16. A method according to claim 11, wherein the pharmaceutical composition is administered by depot injection.

17. A method of treating a disease state related to Lp(a) or LDL cholesterol levels comprising administering to a mammal in need thereof a therapeutically effective amount of a pharmaceutical composition according to claim 8 and optionally, in combination, a therapeutically effective amount of a compound known to be effective for regulating HDL or LDL levels.

18. The method according to claim 17, wherein the mammal is a human.

19. The method according to claim 17, wherein the pharmaceutical composition is administered in a dosage of between about 0.1 mg to about 5000 mg per day.

20. The method according to claim 17, wherein the pharmaceutical composition is administered in a dosage of between about 50 mg to about 500 mg per day.

21. The method according to claim 17, wherein the pharmaceutical composition is administered orally.

22. The method according to claim 17, wherein the pharmaceutical composition is administered as depot injection.

23. The method according to claim 17, wherein the compound known to be effective for regulating HDL or LDL levels is selected from the group consisting of statins, fibrates, bile acid sequestrants, and cholesterol uptake inhibitors.

24. The method according to claim 23, wherein the compound known to be effective for regulating HDL or LDL levels is selected from lovastatin, simvastatin, pravastatin, atorvastatin, cerivastatin, niacin, clofibrate, bezafibrate, gemfibrozil, cholestyramine, phytosteroids, and ezitimibe.

25. A method of lowering blood triglyceride levels comprising administering to a subject in need thereof a therapeutically effective amount of a pharmaceutical composition according to claim 8 and optionally, in combination, a therapeutically effective amount of niacin or a fibrate.

26. A method of treating Syndrome X in a mammal comprising administering to a subject in need thereof a therapeutically effective amount of a pharmaceutical composition according to claim 8 and optionally, in combination, a therapeutically effective amount of a compound selected from the group consisting of rosiglitazone, pioglitazone, insulin, and metformin.

27. A method of treating or preventing atherosclerosis comprising administering to a subject in need thereof a therapeutically effective amount of a pharmaceutical composition according to claim 8.

28. The method of claim 27, further comprising administering a compound known to reduce LDL cholesterol in combination with pharmaceutical composition.

29. A method of treating or preventing thrombosis comprising administering to a subject in need thereof a therapetuically effective amount of a pharmaceutical composition according to claim 8.

30. The method of claim 29, further comprising administering an anti-coagulant in combination with the pharmaceutical composition.

31. A method of regulating apo(a) levels in a mammal comprising administering to a mammal an effective amount of a pharmaceutical composition according to claim 8.

32. A method of regulating apo B levels in a mammal comprising administering to a mammal an effective amount of a pharmaceutical composition according to claim 8.
